(19) United States
(12) Patent Application Publication
(10) Pub. No.: US 2021/0332495 A1
(43) Pub. Date: Oct. 28, 2021

(54) Protein Crystal Engineering Through DNA Hybridization Interactions

(71) Applicant: NORTHWESTERN UNIVERSITY, Evanston, IL (US)

(72) Inventors: Chad A. Mirkin, Wilmette, IL (US); Janet R. McMillan, Evanston, IL (US); Oliver G. Hayes, Evanston, IL (US); Peter H. Winegar, Evanston, IL (US)

(21) Appl. No.: 17/311,108

(22) PCT Filed: Dec. 6, 2019
(86) PCT No.: PCT/US19/65078

§ 371 Date: Jun. 4, 2021

Related U.S. Application Data
(60) Provisional application No. 62/776,399, filed on Dec. 6, 2018.

(51) Int. Cl. C30B 7/14; C07K 1/30; C12P 19/02; B01D 9/00; C30B 29/58

(52) U.S. Cl. CPC C30B 7/14; C07K 1/306; B01D 2009/0086; B01D 9/00; C30B 29/58; C12P 19/02

(57) ABSTRACT

The present disclosure provides compositions comprising protein crystals and methods for programmable biomaterial synthesis. The methods of the disclosure provide the ability to organize proteins within protein crystals with control over protein orientation.

### CROSS-REFERENCE TO RELATED APPLICATIONS

This application claims the priority benefit under 35 U.S.C. § 119(e) of U.S. Provisional Patent Application No. 62/776,399, filed Dec. 6, 2018, which is incorporated herein by reference in their entirety.

### STATEMENT OF GOVERNMENT INTEREST

This invention was made with government support under N00014-15-1-0043, awarded by the Office of Naval Research. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2018-204_Seqlisting.txt”, which was created on Dec. 6, 2019 and is 8,598 bytes in size. The subject matter of the Sequence Listing is incorporated herein in its entirety by reference.

### FIELD OF THE INVENTION

The present disclosure provides compositions comprising protein crystals and methods for programmable biomaterial synthesis.

### BACKGROUND

Chemists routinely design crystals with tunable topology, porosity, and reactive sites. However, structural biologists have not accomplished comparable feats with crystals comprised of biomacromolecules.¹⁻⁴ Protein crystals are a versatile class of materials for catalysis,⁵ protein structure determination,⁶ and separations,⁷ however, they are often grown through trial-and-error approaches, as the complexity of protein-protein interactions (PPIs) limits their rational design.⁸

Protein crystals are an important class of biomaterials, however they are grown almost exclusively through trial-and-error methods and the final structure obtained is not designed, and cannot be controlled. Due to the complexity of protein-protein interactions (PPIs), no current method exists to design the structure of a single protein, or of multiple proteins, within protein crystals.

Through x-ray crystallography, protein single crystals enable fundamental understanding of protein structure and recognition [McRee, D. E. (1999). Practical protein crystallography (Elsevier); Rohs, R., Jin, X., West, S. M., Joshi, R., Honig, B., and Mann, R. S. (2010). Origins of Specificity in Protein-DNA Recognition. Annu. Rev. Biochem. 79, 233-269; Chothia, C., and Janin, J. (1975). Principles of protein—protein recognition. Nature 256, 705-708], and consequently have been important in the rational design of drugs [Mandal, S., Moudgil, M.n., and Mandal, S. K. (2009). Rational drug design. Eur. J. Pharmacol. 625, 90-100]. In addition, they have been used in chiral catalysis [Lalonde, J. J., Govardhan, C., Khalaf, N., Martinez, A. G., Visuri, K., and Margolin, A. L. (1995). Cross-linked crystals of Candida rugosa lipase: highly efficient catalysts for the resolution of chiral esters. J. Am. Chem. Soc. 117, 6845-6852] and enantiomeric separations [Vuolanto, A., Kiviharju, K., Nevanen, T. K., Leisola, M., and Jokela, J. (2003). Development of Cross-Linked Antibody Fab Fragment Crystals for Enantioselective Separation of a Drug Enantiomer. Cryst. Growth Des. 3, 777-782], and non-crystalline but ordered protein assemblies have been utilized to control cascade reactions [Fu, J., Yang, Y. R., Johnson-Buck, A., Liu, M., Liu, Y., Walter, N. G., Woodbury, N. W., and Yan, H. (2014). Multi-enzyme complexes on DNA scaffolds capable of substrate channelling with an artificial swinging arm. Nat. Nanotechnol. 9, 531; Wilner, O. I., Weizmann, Y., Gill, R., Lioubashevski, O., Freeman, R., and Willner, I. (2009). Enzyme cascades activated on topologically programmed DNA scaffolds. Nat. Nanotechnol. 4, 249-254; Niemeyer, C. M., Koehler, J., and Wuerdemann, C. (2002). DNA-Directed Assembly of Bienzymic Complexes from In Vivo Biotinylated NAD(P)H:FMN Oxidoreductase and Luciferase. ChemBioChem 3, 242-245]. However, protein crystallization is challenging because proteins are complex, dynamic molecules comprised of thousands of atoms [McPherson, A., and Gavira, J. A. (2013). Introduction to protein crystallization. Acta Crystallogr., Sect. F: Struct. Biol. Commun. 70, 2-20]. Furthermore, the interactions between protein surfaces that drive crystallization are weak, complex, and noncovalent, therefore, researchers interested in such structures have little control over crystallization and the type of crystals that form [Durbin, S. D., and Feher, G. (1996). Protein Crystallization. Annu. Rev. Phys. Chem. 47, 171-204].

Efforts to control protein crystallization have included modifications that affect charge [Cohen-Hadar, N., Lagziel-Simis, S., Wine, Y., Frolow, F., and Freeman, A. (2011). Re-structuring protein crystals porosity for biotemplating by chemical modification of lysine residues. Biotechnol. Bioeng. 108, 1-11; Simon, A. J., Zhou, Y., Ramasubramani, V., Glaser, J., Pothukuchy, A., Gollihar, J., Gerberich, J. C., Leggere, J. C., Morrow, B. R., Jung, C., et al. (2019). Supercharging enables organized assembly of synthetic biomolecules. Nat. Chem. 11, 204-212; Küunzle, M., Eckert, T., and Beck, T. (2016). Binary Protein Crystals for the Assembly of Inorganic Nanoparticle Superlattices. J. Am. Chem. Soc. 138, 12731-12734], hydrophobicity [Yamada, H., Tamada, T., Kosaka, M., Miyata, K., Fujiki, S., Tano, M., Moriya, M., Yamanishi, M., Honjo, E., Tada, H., et al. (2007). ‘Crystal lattice engineering,’ an approach to engineer protein crystal contacts by creating intermolecular symmetry: crystallization and structure determination of a mutant human RNase 1 with a hydrophobic interface of leucines. Protein Sci. 16, 1389-1397], protein structure [King, N. P., Bale, J. B., Sheffler, W., McNamara, D. E., Gonen, S., Gonen, T., Yeates, T. O., and Baker, D. (2014). Accurate design of co-assembling multi-component protein nanomaterials. Nature 510, 103-108; Brunette, T. J., Parmeggiani, F., Huang, P.-S., Bhabha, G., Ekiert, D. C., Tsutakawa, S. E., Hura, G. L., Tainer, J. A., and Baker, D. (2015). Exploring the repeat protein universe through computational protein design. Nature 528, 580-584; Doyle, L., Hallinan, J., Bolduc, J., Parmeggiani, F., Baker, D., Stoddard, B. L., and Bradley, P. (2015). Rational design of a-helical tandem repeat proteins with closed architectures. Nature 528, 585-588], ligand binding [Engilberge, S., Rennie, M. L., Dumont, E., and Crowley, P. B. (2019). Tuning Protein Frameworks via Auxiliary Supramolecular Interactions. ACS Nano 13, 10343-10350; Alex, J. M., Rennie, M. L., Volpi, S., Sansone, F., Casnati, A., and Crowley, P. B. (2018). Phosphonated Calixarene as a “Molecular Glue” for Protein Crystallization. Cryst. Growth Des. 18, 2467-2473; Sakai, F., Yang, G., Weiss, M. S., Liu, Y., Chen, G., and Jiang, M. (2014). Protein crystalline frameworks with controllable interpenetration directed by dual supramolecular interactions. Nat. Commun. 5, 4634; Rennie, M. L., Fox, G. C., Perez, J., and Crowley, P. B. (2018). Auto-regulated Protein Assembly on a Supramolecular Scaffold. Angew. Chem. 130, 13960-13965], and metal binding characteristics [Lawson, D. M., Artymiuk, P. J., Yewdall, S. J., Smith, J. M. A., Livingstone, J. C., Treffry, A., Luzzago, A., Levi, S., Arosio, P., Cesareni, G., et al. (1991). Solving the structure of human H ferritin by genetically engineering intermolecular crystal contacts. Nature 349, 541-544; Brodin, J. D., Ambroggio, X. I., Tang, C., Parent, K. N., Baker, T. S., and Tezcan, F. A. (2012). Metal-directed, chemically tunable assembly of one-, two- and three-dimensional crystalline protein arrays. Nat. Chem. 4, 375-382; Sontz, P. A., Bailey, J. B., Ahn, S., and Tezcan, F. A. (2015). A Metal Organic Framework with Spherical Protein Nodes: Rational Chemical Design of 3D Protein Crystals. J. Am. Chem. Soc. 137, 11598-11601], and they often involve the introduction of functional groups via site-directed mutagenesis [Derewenda, Z. (2010). Application of protein engineering to enhance crystallizability and improve crystal properties. Acta Crystallogr., Sect. D: Biol. Crystallogr. 66, 604-615; McPherson, A. (2017). Protein Crystallization. In Protein Crystallography: Methods and Protocols, A. Wlodawer, Z. Dauter, and M. Jaskolski, eds. (Springer New York), pp. 17-50]. In 2015, the concept of DNA-modification to control protein crystallization was introduced [Brodin, J. D., Auyeung, E., and Mirkin, C. A. (2015). DNA-mediated engineering of multicomponent enzyme crystals. Proc. Natl. Acad. Sci. U. S. A. 112, 4564-4569]. With isotropically and sometimes anisotropically functionalized structures, pseudo-crystalline materials could be realized, but to date, these techniques have not yielded structures suitable for single-crystal x-ray diffraction studies [Brodin, J. D., Auyeung, E., and Mirkin, C. A. (2015). DNA-mediated engineering of multicomponent enzyme crystals. Proc. Natl. Acad. Sci. U. S. A. 112, 4564-4569; Hayes, O. G., McMillan, J. R., Lee, B., and Mirkin, C. A. (2018). DNA-Encoded Protein Janus Nanoparticles. J. Am. Chem. Soc. 140, 9269-9274; McMillan, J. R., Brodin, J. D., Millan, J. A., Lee, B., Olvera de la Cruz, M., and Mirkin, C. A. (2017). Modulating Nanoparticle Superlattice Structure Using Proteins with Tunable Bond Distributions. J. Am. Chem. Soc. 139, 1754-1757; Subramanian, R. H., Smith, S. J., Alberstein, R. G., Bailey, J. B., Zhang, L., Cardone, G., Suominen, L., Chami, M., Stahlberg, H., Baker, T. S., et al. (2018). Self-Assembly of a Designed Nucleoprotein Architecture through Multimodal Interactions. ACS Cent. Sci. 4, 1578-1586; Mirkin, C. A., Letsinger, R. L., Mucic, R. C., and Storhoff, J. J. (1996). A DNA-based method for rationally assembling nanoparticles into macroscopic materials. Nature 382, 607-609; Park, S. Y., Lytton-Jean, A. K. R., Lee, B., Weigand, S., Schatz, G. C., and Mirkin, C. A. (2008). DNA-programmable nanoparticle crystallization. Nature 451, 553-556; McMillan, J. R., Hayes, O. G., Winegar, P. H., and Mirkin, C. A. (2019). Protein Materials Engineering with DNA. Acc. Chem. Res. 52, 1939-1948; McMillan, J. R., and Mirkin, C. A. (2018). DNA-Functionalized, Bivalent Proteins. J. Am. Chem. Soc. 140, 6776-6779].

### SUMMARY

The present disclosure addresses the foregoing challenges by introducing a well-defined number of DNA ligands conjugated to precise locations on protein surfaces to control macromolecular structure during crystallization, where both DNA hybridization interactions and PP Is will contribute to the overall structure observed. This method enables the structure of protein crystals to be programmed and controlled for the first time. Using the methods of the disclosure, protein crystal structure is controlled through programming DNA sequence, length, and placement. Experiments that are partially described herein demonstrated that the structure of a protein crystal can be modulated based off of the placement of a single DNA modification on its surface, and that the sequence of this DNA modification alters structural outcome.

The design space for DNA ligands that are amenable to protein crystallization are also mapped out, how these ligands affect protein crystal structure are elucidated. This information enables more complex systems to be designed, where multiple proteins with complementary functions are incorporated into a single crystal, and architectural parameters such as protein orientation and porosity are finely controlled.

Applications of the technology disclosed herein include, but are not limited to, the following.

Protein structural determination

Synthesis of multi-component protein crystals with tunable structure

Synthesis of highly porous protein crystals

Cascade biocatalysis

Enantiomeric separations

Separations involving protein ligands

The compositions and methods of the disclosure also provide several advantages, which include the fact that the DNA ligands have a designable length and bond strength, and that the DNA hybridization interaction is independent of protein identity.

Analogous to modular metal-ligand interactions which have enabled the structure and properties of metal-organic framework (MOF) crystals to be rationally tuned,⁹ introducing ligands onto protein surfaces to mediate their crystallization is disclosed herein as enabling rational design of these materials. The ligands of choice include, but are not limited to, oligonucleotides due to their versatile chemistry.

In contrast to PPIs, robust solid-phase DNA synthesis enables programmable DNA design with arbitrary sequence and length. The thermodynamic preference for Watson-Crick base pair interactions (adenine with thymine and cytosine with guanine) provides a rational way to design almost unlimited orthogonal DNA hybridization interactions, something highly challenging and computationally intensive to achieve with PPIs.¹⁰ Moreover, DNA architectures, such as helical junctions and three-dimensional shapes, are routinely designed from ensembles of DNA sequences to organize nanoscale materials.¹¹⁻¹³ Previous work has described colloidal crystallization strategies using DNA to direct the assembly of nanoparticles.^{14,15} Recent studies apply this expertise towards developing new protein-based materials, using DNA to control protein co-crystallization with nanoparticles,¹⁶⁻¹⁸ and protein polymerization.^{19,20} While the aforementioned examples exclusively use DNA hybridization interactions to direct the organization of proteins, the present disclosure synergizes PPIs with DNA interactions to control the crystallization of proteins. Through the conjugation of one or two or more short oligonucleotides to a protein's surface, protein crystallization is driven by both native PPIs and the design of the DNA sequence, resulting in the ability to finely control structure. Specifically, the present disclosure enables tunable symmetry, topology, porosity and reactive site orientation in protein crystals, leading to applications in, for example and without limitation, heterogeneous cascade catalysis, protein structure determinations and chiral separations. Predictable and programmable protein crystallization, therefore, represents a major advance in the understanding and synthesis of materials in the bio-material space.

Accordingly, in some aspects the disclosure provides a method of producing a protein crystal comprising contacting a first conjugate comprising a first protein and a first polynucleotide with a second conjugate comprising a second protein and a second polynucleotide under conditions sufficient such that the first polynucleotide and the second polynucleotide hybridize to each other and the first protein and second protein associate via protein-protein interactions (PPI) to form the protein crystal. In some embodiments, the first protein and the second protein are the same. In some embodiments, the first protein and the second protein are different. In further embodiments, the first polynucleotide is from about 2 to about 30 nucleotides in length. In still further embodiments, the second polynucleotide is from about 2 to about 30 nucleotides in length. In any of the aspects or embodiments of the disclosure, the first polynucleotide is DNA. In any of the aspects or embodiments of the disclosure, the second polynucleotide is DNA. In some embodiments, the first protein consists of one polynucleotide that is sufficiently complementary to one or more polynucleotides on the second protein to hybridize. In further embodiments, the first protein comprises one polynucleotide that is sufficiently complementary to one or more polynucleotides on the second protein to hybridize. In some embodiments, the first protein consists of two, three, four, or five polynucleotides that are sufficiently complementary to one or more polynucleotides on the second protein to hybridize. In some embodiments, the first protein comprises two, three, four, or five polynucleotides that are sufficiently complementary to one or more polynucleotides on the second protein to hybridize. In some embodiments, the second protein consists of one polynucleotide that is sufficiently complementary to one or more polynucleotides on the first protein to hybridize. In some embodiments, the second protein comprises one polynucleotide that is sufficiently complementary to one or more polynucleotides on the first protein to hybridize. In further embodiments, the second protein consists of two, three, four, or five polynucleotides that are sufficiently complementary to one or more polynucleotides on the first protein to hybridize. In further embodiments, the second protein comprises two, three, four, or five polynucleotides that are sufficiently complementary to one or more polynucleotides on the first protein to hybridize. In some embodiments, the PPI is a hydrophobic bond, van der Waals forces, a salt bridge, a disulfide bond, an electrostatic interaction, hydrogen bonding, or a combination thereof. In some embodiments, the protein crystal is from about 250 nanometer (nm) to about 1 millimeter (mm). In further embodiments, the protein crystal is from about 20 micrometers (μm) to about 500 μm in edge length. In some embodiments, the structure of the protein crystal diffracts to angstrom level resolution. In some embodiments, the first polynucleotide is attached to the N-terminus of the first protein. In further embodiments, the first polynucleotide is attached to the C-terminus of the first protein. In still further embodiments, the first polynucleotide is attached to the N-terminus of the first protein and a second polynucleotide is attached to the C-terminus of the first protein. In yet additional embodiments, the first polynucleotide is attached to the N-terminus of the first protein, a second polynucleotide is attached to the C-terminus of the first protein, and a third polynucleotide is attached to the first protein between the N-terminus and the C-terminus. In some embodiments, the second polynucleotide is attached to the N-terminus of the second protein. In further embodiments, the second polynucleotide is attached to the C-terminus of the second protein. In still further embodiments, the second polynucleotide is attached to the N-terminus of the second protein and a second polynucleotide is attached to the C-terminus of the second protein. In yet additional embodiments, the first polynucleotide is attached to the N-terminus of the second protein, a second polynucleotide is attached to the C-terminus of the second protein, and a third polynucleotide is attached to the second protein between the N-terminus and the C-terminus. In some embodiments, the first polynucleotide is attached to the first protein via an unnatural amino acid introduced into the first protein via mutation. In some embodiments, the second polynucleotide is attached to the second protein via an unnatural amino acid introduced into the second protein via mutation. In further embodiments, the first polynucleotide is attached to the first protein via a surface amino group of the first protein. In some embodiments, the second polynucleotide is attached to the second protein via a surface amino group of the second protein. In some embodiments, the surface amino group is from a Lys residue. In further embodiments, the first polynucleotide is attached to the first protein via a triazole linkage formed from reaction of (a) an azide moiety attached to the surface amino group and (b) an alkyne functional group on the first polynucleotide. In some embodiments, the second polynucleotide is attached to the second protein via a triazole linkage formed from reaction of (a) an azide moiety attached to the surface amino group and (b) an alkyne functional group on the second polynucleotide. In some embodiments, the first polynucleotide is attached to the first protein via a surface carboxyl group of the first protein. In further embodiments, the second polynucleotide is attached to the second protein via a surface carboxyl group of the second protein. In some embodiments, the first polynucleotide is attached to the first protein via a surface thiol group of the first protein. In further embodiments, the second polynucleotide is attached to the second protein via a surface thiol group of the second protein. In various embodiments, the protein crystal exhibits catalytic, signaling, therapeutic, or transport activity. In some embodiments, the first protein and/or the second protein is a protein fragment. In some embodiments, the contacting step further comprises contacting the first conjugate and/or the second conjugate with a third conjugate comprising a third protein and a third polynucleotide, wherein the third polynucleotide hybridizes to the first polynucleotide or the second polynucleotide, and the resulting protein crystal comprises the first protein, second protein, and third protein. In further embodiments, the protein crystal has a pore size of from about 1 nanometer (nm) to about 100 nm in diameter.

In some aspects, the disclosure provides a protein crystal comprising a first conjugate and a second conjugate, wherein the first conjugate comprises a first protein and a first polynucleotide and the second conjugate comprises a second protein and a second polynucleotide, wherein the first polynucleotide and the second polynucleotide are sufficiently complementary to hybridize to each other. In some embodiments, the first protein and the second protein are the same. In further embodiments, the first protein and the second protein are different. In some embodiments, the first polynucleotide is from about 2 to about 30 nucleotides in length. In some embodiments, the second polynucleotide is from about 2 to about 30 nucleotides in length. In some embodiments, the first protein consists of one, two, three, four, or five polynucleotides that are sufficiently complementary to one or more polynucleotides on the second protein to hybridize. In some embodiments, the first protein comprises one, two, three, four, or five polynucleotides that are sufficiently complementary to one or more polynucleotides on the second protein to hybridize. In some embodiments, the first protein consists of one polynucleotide that is sufficiently complementary to one or more polynucleotides on the second protein to hybridize. In some embodiments, the first protein comprises one polynucleotide that is sufficiently complementary to one or more polynucleotides on the second protein to hybridize. In some embodiments, the second protein consists of one, two, three, four, or five polynucleotides that are sufficiently complementary to one or more polynucleotides on the first protein to hybridize. In some embodiments, the second protein comprises one, two, three, four, or five polynucleotides that are sufficiently complementary to one or more polynucleotides on the first protein to hybridize. In some embodiments, the second protein consists of one polynucleotide that is sufficiently complementary to one or more polynucleotides on the first protein to hybridize. In some embodiments, the second protein comprises one polynucleotide that is sufficiently complementary to one or more polynucleotides on the first protein to hybridize. In some embodiments, the first protein and the second protein associate with each other through a protein-protein interaction (PPI). In some embodiments, the PPI is a hydrophobic bond, van der Waals forces, a salt bridge, a disulfide bond, an electrostatic interaction, hydrogen bonding, or a combination thereof. In further embodiments, the protein crystal is from about 250 nanometer (nm) to about 1 millimeter (mm), or from about 20 micrometers (pm) to about 500 μm in edge length. In some embodiments, the structure of the protein crystal diffracts to angstrom level resolution. In further embodiments, the first polynucleotide is attached to the N-terminus of the first protein. In some embodiments, the first polynucleotide is attached to the C-terminus of the first protein. In still further embodiments, the first polynucleotide is attached to the N-terminus of the first protein and a second polynucleotide is attached to the C-terminus of the first protein. In yet additional embodiments, the first polynucleotide is attached to the N-terminus of the first protein, a second polynucleotide is attached to the C-terminus of the first protein, and a third polynucleotide is attached to the first protein between the N-terminus and the C-terminus. In further embodiments, the second polynucleotide is attached to the N-terminus of the second protein. In further embodiments, the second polynucleotide is attached to the C-terminus of the second protein. In still further embodiments, the second polynucleotide is attached to the N-terminus of the second protein and a second polynucleotide is attached to the C-terminus of the second protein. In yet additional embodiments, the first polynucleotide is attached to the N-terminus of the second protein, a second polynucleotide is attached to the C-terminus of the second protein, and a third polynucleotide is attached to the second protein between the N-terminus and the C-terminus. In some embodiments, the first polynucleotide is attached to the first protein via an unnatural amino acid introduced into the first protein via mutation. In further embodiments, the second polynucleotide is attached to the second protein via an unnatural amino acid introduced into the second protein via mutation. In some embodiments, the first polynucleotide is attached to the first protein via a surface amino group of the first protein. In further embodiments, the second polynucleotide is attached to the second protein via a surface amino group of the second protein. In some embodiments, the surface amino group is from a Lys residue. In some embodiments, the first polynucleotide is attached to the first protein via a triazole linkage formed from reaction of (a) an azide moiety attached to the surface amino group and (b) an alkyne functional group on the first polynucleotide. In some embodiments, the second polynucleotide is attached to the second protein via a triazole linkage formed from reaction of (a) an azide moiety attached to the surface amino group and (b) an alkyne functional group on the second polynucleotide. In further embodiments, the first polynucleotide is attached to the first protein via a surface carboxyl group of the first protein. In some embodiments, the second polynucleotide is attached to the second protein via a surface carboxyl group of the second protein. In further embodiments, the first polynucleotide is attached to the first protein via a surface thiol group of the first protein. In some embodiments, the second polynucleotide is attached to the second protein via a surface thiol group of the second protein. In further embodiments, the protein crystal exhibits catalytic, signaling, therapeutic, or transport activity. In some embodiments, the first protein and/or the second protein is a protein fragment. In further embodiments, the protein crystal further comprises a third conjugate comprising a third protein and a third polynucleotide, wherein the third polynucleotide is sufficiently complementary to the first polynucleotide or the second polynucleotide to hybridize. In some embodiments, the protein crystal has a pore size of from about 1 nanometer (nm) to about 100 nm in diameter.

In some aspects, the disclosure provides a method of catalyzing a reaction comprising contacting one or more reagents for the reaction with the protein crystal of any one of claims 30-57, wherein contact between the reagents and the protein crystal results in the reaction being catalyzed to form a product of the reaction.

### BRIEF DESCRIPTION OF THE FIGURES

FIG. 1 shows a schematic of project workflow. (A) A single amine-terminated DNA was conjugated to GFP with a succinimidyl 3-(2-pyridyldithio)propionate (SPDP) cross linker. (B) With correct DNA design, (C) DNA hybridization interactions could be programmed between GFP-DNA conjugates. (D) Crystallization screens were used to search for crystals from these conjugates.

FIG. 2 depicts characterization data for GFP-DNA conjugates after purification. (A) MS-MALDI characterization. (B) SDS-PAGE characterization.

FIG. 3 shows protein crystal structures for i. GFP, ii. GFP-nc6mer, and iii. GFP-sc6mer. (A) Optical micrographs of protein crystals. (B) Selected diffraction pattern for these crystals. (C) Preliminary crystal structure with multiple asymmetric units shown. (D) Space group and unit cell dimensions. Results of a further analysis of some of the data in FIG. 3 are presented in FIGS. 27, 28, 39c, and Table 4.

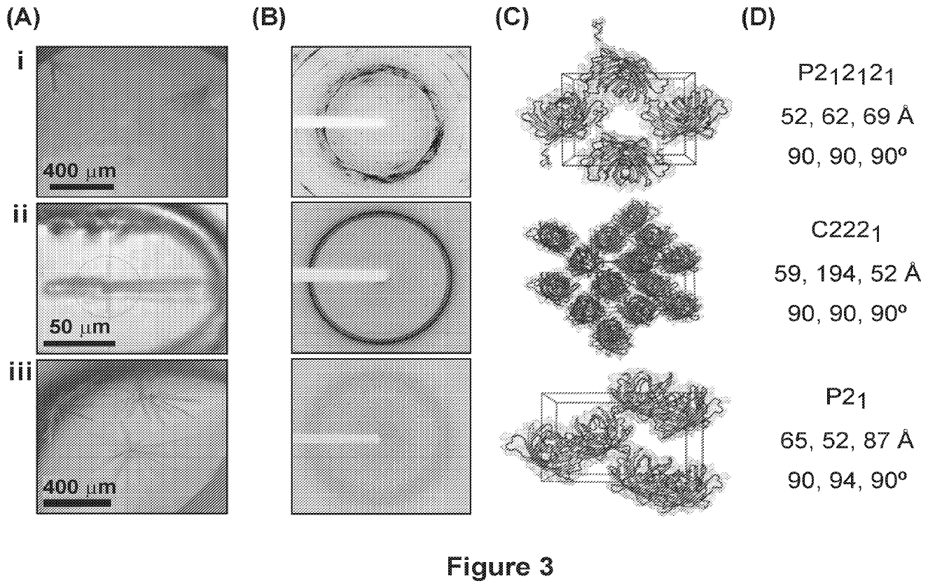

FIG. 4 shows depicts the introduction of a DNA ligand onto a protein's surface to control protein crystal structure. (a) DNA may be conjugated specifically to N-terminal amines or mutated surface residues, such as cysteines. (b) Interactions between proteins can be designed by modifying DNA complementarity, DNA length and DNA conjugation sites, (c) leading to crystal growth with tunable space group, protein packing and crystal contacts.

FIG. 5 demonstrates that DNA ligands may program co-crystallization of distinct proteins, (a) For the model system of GFP and MBP, conjugation of multiple orthogonal DNA sequences (b) may enable protein frameworks with tunable porosity and (c) designable architecture, analogous to MOFs. (d) Organizing i. 8-galactosidase, ii. hexokinase and iii. glucose-6-phosphate dehydrogenase with DNA ligands may lead to increased catalytic rates of a three-step conversion from lactose to an oxidized, phosphorylated glucose.

FIG. 6. SDS PAGE Confirms Purity of mGFP Mutants. SDS PAGE analysis showed that mGFP mutants was expressed and purified for (A) C148 mGFP, (B) C176 mGFP, and (C) C191 mGFP. The majority of mGFP mutants are monomeric (˜30 kDa) with surface cysteine residues as thiols (reduced). A fraction of mGFP mutants formed dimers (˜60 kDa) through the formation of disulfide bonds (oxidized).

FIG. 7 shows linkage structures of mGFP-DNA conjugates. Linkage structure for mGFP-DNA conjugates with (A) external and (B) internal DNA attachment positions. Atoms from mGFP and DNA are colored in blue and atoms from SPDP are colored in black.

FIG. 8 shows confocal microscopy images of C148 mGFP crystals. Five crystals of C148 mGFP (labeled 1-5) were imaged with a bright field (left), a green channel (middle, 485 nm excitation and 500-550 nm emission filter), and a far-red channel (right, 640 nm excitation and 663-738 nm emission filter) (A) before and (B) 30 min after addition of a DNA intercalating dye. The ratio of green to far-red signal intensity from selected areas of the images was 108±57 before the dye addition and 10.3±4.7 after the dye addition. Scale bars are 50 μm.

FIG. 9 shows confocal microscopy images of C148 mGFP-ncDNA-1 crystals. Three crystals of C148 mGFP-ncDNA-1 (labeled 1-3) were imaged with a bright field (left), a green channel (middle, 485 nm excitation and 500-550 nm emission filter), and a far-red channel (right, 640 nm excitation and 663-738 nm emission filter) (A) before and (B) 30 min after addition of a DNA intercalating dye. The ratio of green to far-red signal intensity from selected areas of the images was 96±20 before the dye addition and 1.8±0.7 after the dye addition. Scale bars are 50 μm.

FIG. 10 shows confocal microscopy images of C148 mGFP-cDNA-1 crystals. Four crystals of C148 mGFP-cDNA-1 (labeled 1-4) were imaged with a bright field (left), a green channel (middle, 485 nm excitation and 500-550 nm emission filter), and a far-red channel (right, 640 nm excitation and 663-738 nm emission filter) (A) before and (B) 30 min after addition of a DNA intercalating dye. The ratio of green to far-red signal intensity from selected areas of the images was 124±40 before the dye addition and 1.4±0.4 after the dye addition. Scale bars are 50 μm.

FIG. 11 shows Characterization of C148 mGFP. (A) Schematic of C148 mGFP (green) in the thiol form with the surface cysteine location marked in blue. (B) A UV-vis absorption spectrum that is normalized to the C148 mGFP (green) chromophore absorbance at 488 nm. A second absorbance at 280 nm is due to aromatic amino acid side chains. (C) SDS PAGE analysis shows C148 mGFP (lane 1, green) primarily in the thiol form (˜30 kDA) with a small amount in the disulfide form (˜60 kDa). (D) Mass characterization using MALDI-MS shows the experimental C148 mGFP (green) mass of ˜30.5 kDa.

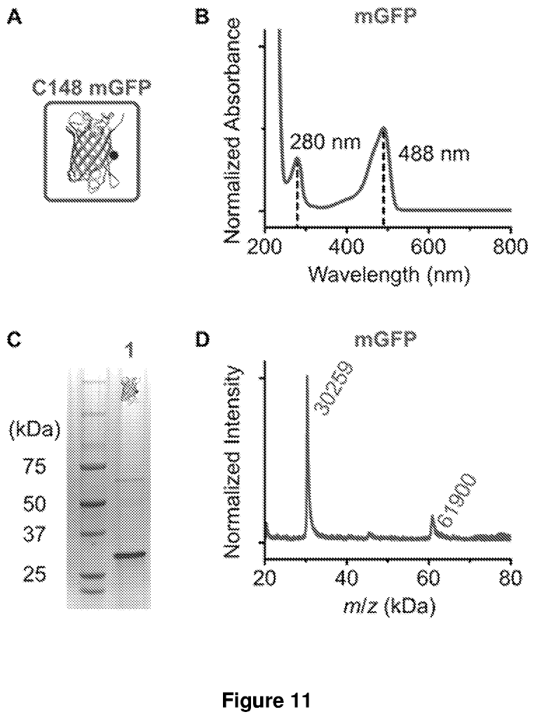

FIG. 12 shows the packing arrangement of the C148 mGFP crystal structure (6UHJ). The packing arrangement of C148 mGFP (colored in teal with surface cysteines colored in red) in the C148 mGFP crystal structure.

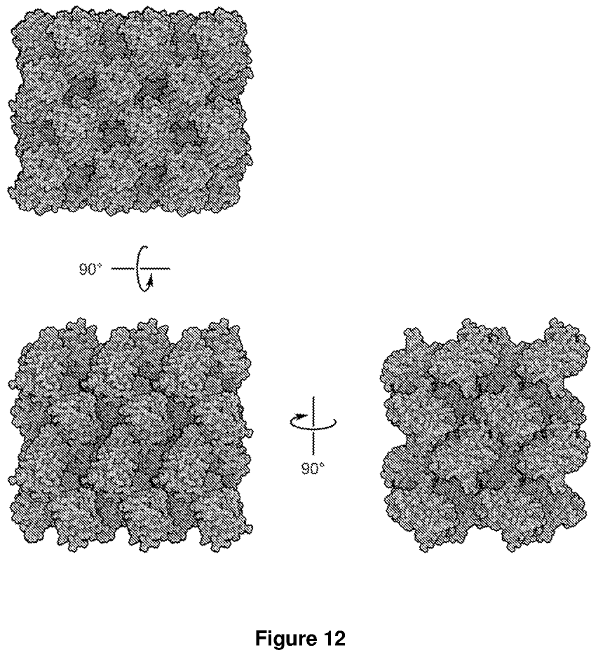

FIG. 13 shows Characterization of C176 mGFP. (A) Schematic of C176 mGFP (green) in the thiol form with the surface cysteine location marked in blue. (B) A UV-vis absorption spectrum that is normalized to the C176 mGFP (green) chromophore absorbance at 488 nm. A second absorbance at 280 nm is due to aromatic amino acid side chains. (C) SDS PAGE analysis shows C176 mGFP (lane 1, green) primarily in the thiol form (˜30 kDA) with a small amount in the disulfide form (˜60 kDa). (D) Mass characterization using MALDI-MS shows experimental C176 mGFP (green) masses of ˜29.0 and 30.5 kDa.

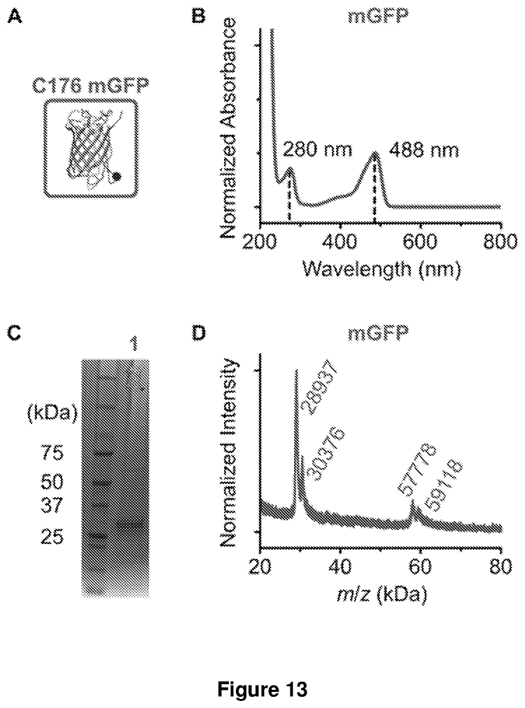

FIG. 14 shows the packing arrangement of the C176 mGFP crystal structure (6UHK). The packing arrangement of C176 mGFP (the two proteins in the asymmetric unit are teal and green with surface cysteines colored in red) in the C176 mGFP crystal structure.

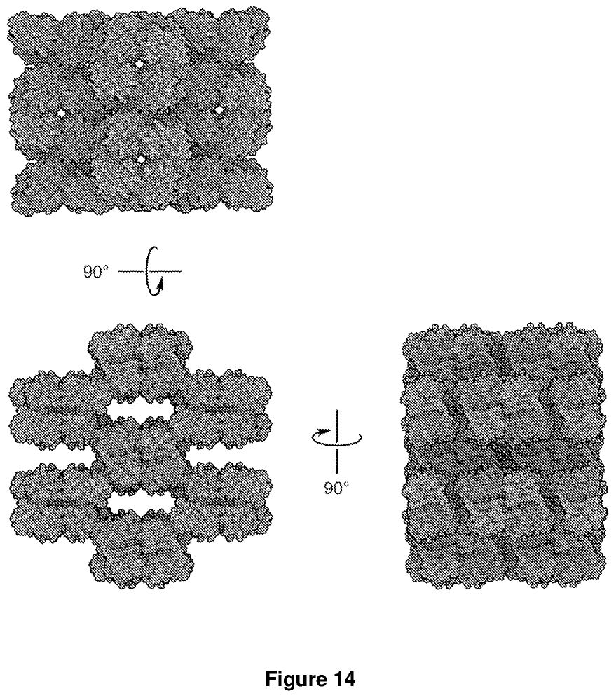

FIG. 15 shows the crystal structure of C176 mGFP as disulfide dimers (6UHK). (A) Schematic of C176 mGFP (green) with the surface cysteine location marked in blue. (B) Subset of the C176 mGFP crystal structure highlighting the disulfide interaction between surface cysteine resides in blue.

FIG. 16 shows the characterization of C191 mGFP. (A) Schematic of C191 mGFP (green) in the thiol form with the surface cysteine location marked in blue. (B) A UV-vis absorption spectrum that is normalized to the C191 mGFP (green) chromophore absorbance at 488 nm. A second absorbance at 280 nm is due to aromatic amino acid side chains. (C) SDS PAGE analysis shows C191 mGFP (lane 1, green) primarily in the thiol form (˜30 kDA) with a small amount in the disulfide form (˜60 kDa). (D) Mass characterization using MALDI-MS shows the experimental C191 mGFP (green) mass of ˜30.5 kDa.

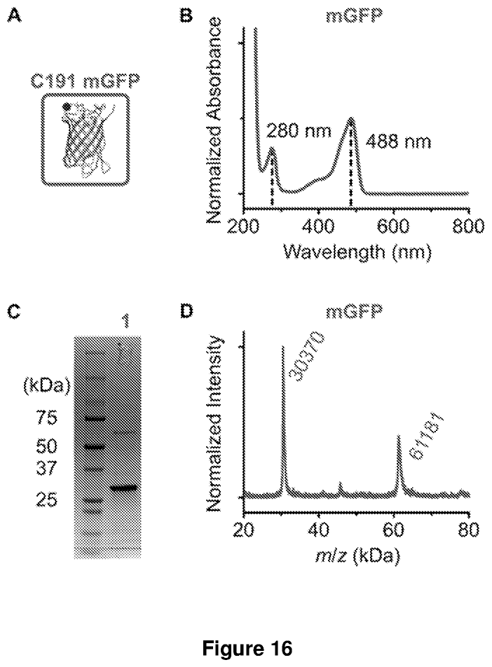

FIG. 17 shows the characterization of C148 mGFP-scDNA-1 conjugates. (A) Schematic of C148 mGFP (green) with the surface cysteine location marked in blue and schematic of C148 mGFP-scDNA-1 (blue) depicting the DNA interaction between C148 mGFP-scDNA-1 conjugates. (B) A UV-vis absorption spectrum that is normalized to the C148 mGFP (green) and C148 mGFP-scDNA-1 (blue) chromophore absorbances at 488 nm. The increase in absorbance at 260 nm in C148 mGFP-scDNA-1 relative to C148 mGFP corresponds to the presence of 1.2 scDNA-1 per C148 mGFP in solution. (C) SDS PAGE analysis shows a mass increase from C148 mGFP (lane 1, green) to C148 mGFP-scDNA-1 (lane 2, blue) that corresponds to conjugation of a single scDNA-1 to C148 mGFP. The single band (˜32 kDa) for C148 mGFP-scDNA-1 indicates high purity. Both images are from the same gel, with intermediate lanes removed for clarity. (D) Mass characterization using MALDI-MS shows a mass increase of 1802 Da from C148 mGFP (green) to C148 mGFP-scDNA-1 (blue) that is consistent with a theoretical mass increase of 2016 Da (1930 Da (scDNA-1)+86 Da (linker)) for the functionalization of C148 mGFP with one strand of scDNA-1.

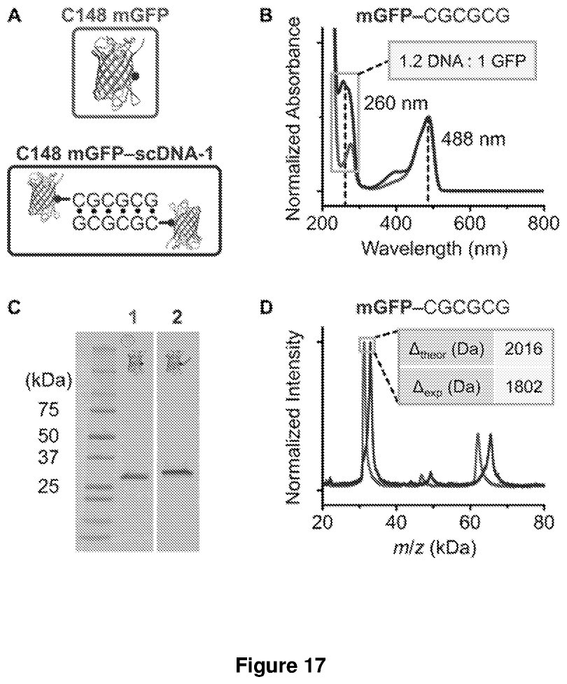

FIG. 18 shows the packing arrangement of the C148 mGFP-scDNA-1 crystal structure (6UHL). The packing arrangement of C148 mGFP (the two proteins in the asymmetric unit are teal and green with surface cysteines colored in red) in the C148 mGFP-scDNA-1 crystal structure.

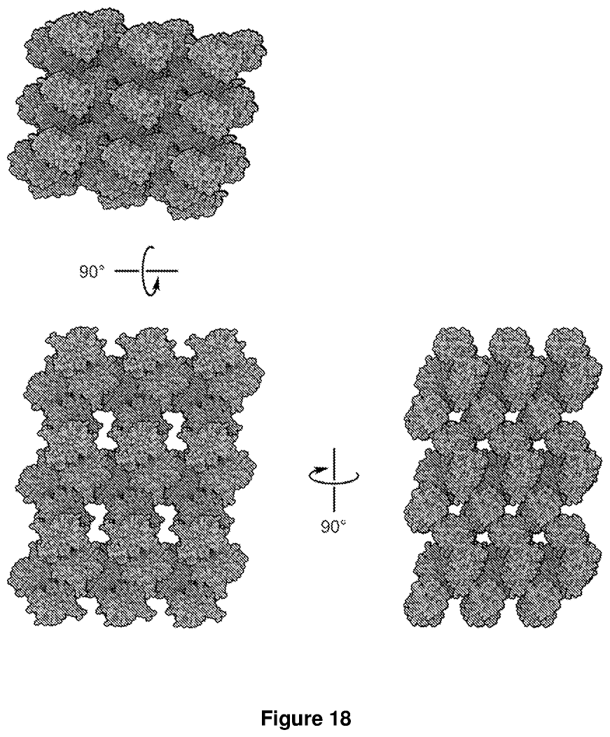

FIG. 19 shows the packing arrangement of the C148 mGFP +scDNA-1 crystal structure (6UHM). The packing arrangement of C148 mGFP (the two proteins in the asymmetric unit are teal and green with surface cysteines colored in red) in the C148 mGFP +scDNA-1 crystal structure.

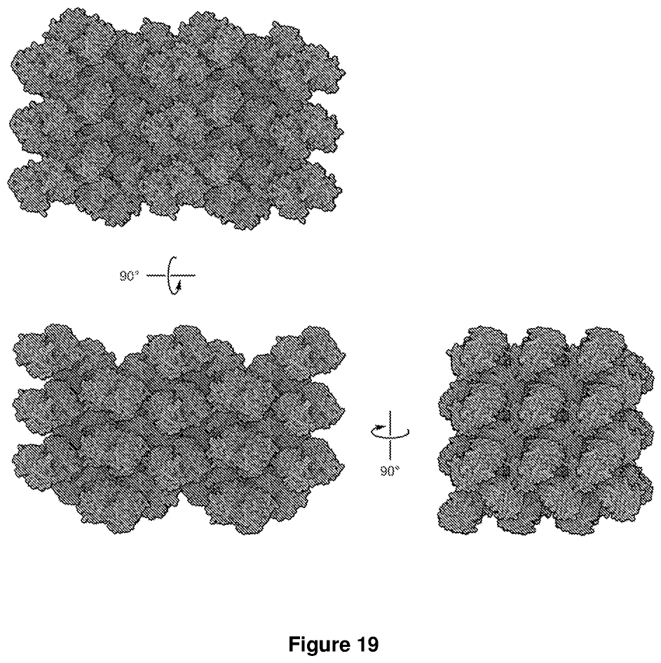

FIG. 20 shows the crystal structure of the physical mixture of C148 mGFP+scDNA-1 as disulfide dimers (6UHM). (A) Schematic of the physical mixture of C148 mGFP and scDNA-1 (blue). (B) Subset of the crystal structure of the physical mixture of C148 mGFP and scDNA-1 highlighting the disulfide interaction between surface cysteine resides in blue.

FIG. 21 shows the characterization of C148 mGFP-cDNA-1 conjugates. (A) Schematic of C148 mGFP (green) with the surface cysteine location marked in blue and schematic of C148 mGFP-cDNA-1 (red and purple for complementary DNA strands) depicting the DNA interaction between C148 mGFP-cDNA-1 conjugates. (B) UV-vis absorption spectra that are normalized to the C148 mGFP (green) and C148 mGFP-cDNA-1 (red or purple) chromophore absorbances at 488 nm. The increase in absorbance at 260 nm in C148 mGFP-cDNA-1 relative to C148 mGFP corresponds to the presence of 1.0 (red DNA design) and 1.1 (purple DNA design) cDNA-1 per C148 mGFP in solution. (C) SDS PAGE analysis shows a mass increase from C148 mGFP (lane 1, green) to C148 mGFP-cDNA-1 (lane 2, red and lane 3, purple) that corresponds to conjugation C148 mGFP one cDNA-1 for each complementary DNA strand. The primary band for each C148 mGFP-cDNA-1 (˜32 kDa) corresponds to C148 mGFP functionalized to a single cDNA-1 strand. Weak secondary bands at ˜30 and ˜60 kDa correspond to small impurities of C148 mGFP in the thiol and disulfide forms, respectively. (D) Mass characterization using MALDI-MS show mass increases of 2002 and 1942 Da from C148 mGFP (green) to each C148 mGFP-cDNA-1 (red and purple, respectively) that is consistent with theoretical mass increases of 2098 (2012 Da (cDNA-1)+86 Da (linker)) and 2018 Da (1932 Da (cDNA-1)+86 Da (linker)), respectively, for the functionalization of C148 mGFP with one strand of cDNA-1.

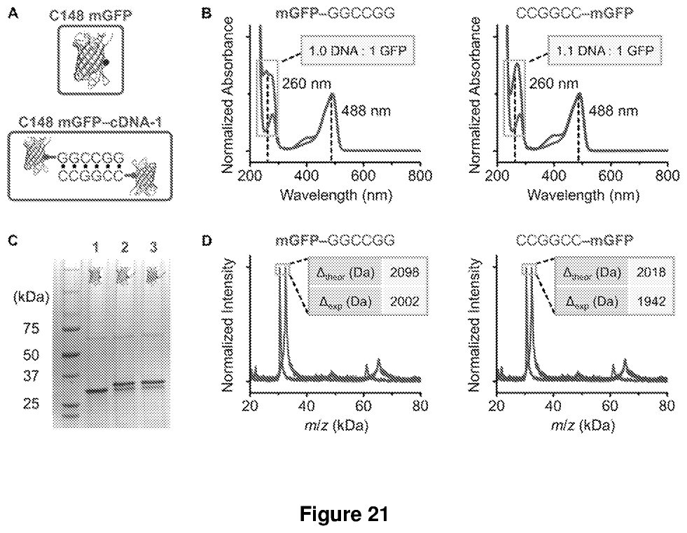

FIG. 22 shows the packing arrangement of the C148 mGFP-cDNA-1 crystal structure (6UHN). The packing arrangement of C148 mGFP (the two proteins in the asymmetric unit are teal and green with surface cysteines colored in red) in the C148 mGFP-cDNA-1 crystal structure.

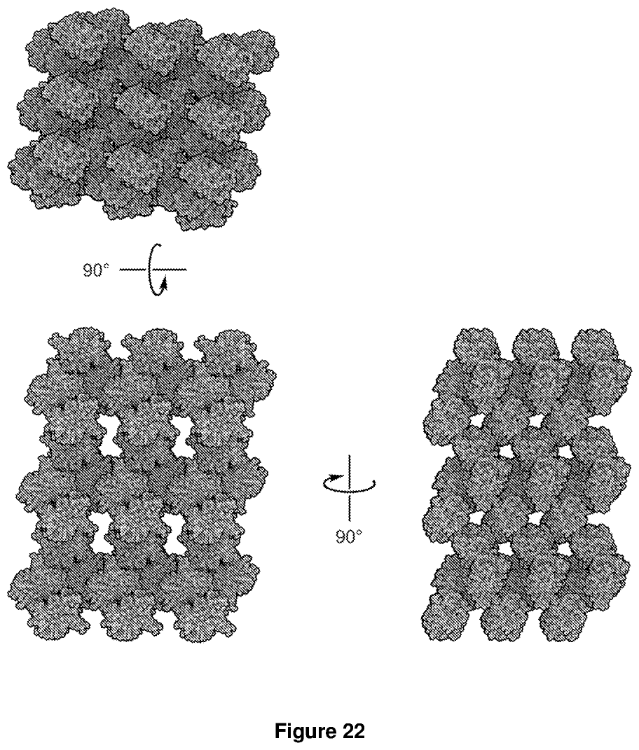

FIG. 23 shows the characterization of C148 mGFP-cDNA-2 conjugates. (A) Schematic of C148 mGFP (green) with the surface cysteine location marked in blue and schematic of C148 mGFP-cDNA-2 (red and purple for complementary DNA strands) depicting the DNA interaction between C148 mGFP-cDNA-2 conjugates. (B) UV-vis absorption spectra that are normalized to the C148 mGFP (green) and C148 mGFP-cDNA-2 (red or purple) chromophore absorbances at 488 nm. The increase in absorbance at 260 nm in C148 mGFP-cDNA-2 relative to C148 mGFP corresponds to the presence of 0.9 (red DNA design) and 1.3 (purple DNA design) cDNA-2 per C148 mGFP in solution. (C) SDS PAGE analysis shows a mass increase from C148 mGFP (lane 1, green) to C148 mGFP-cDNA-2 (lane 2, red and lane 3, purple) that corresponds to conjugation C148 mGFP one cDNA-2 for each complementary DNA strand. The primary band for each C148 mGFP-cDNA-2 (˜32 kDa) corresponds to C148 mGFP functionalized to a single cDNA-2 strand. Weak secondary bands at ˜30 and ˜60 kDa correspond to small impurities of C148 mGFP in the thiol and disulfide forms, respectively. (D) Mass characterization using MALDI-MS show mass increases of 2132 and 1889 Da from C148 mGFP (green) to each C148 mGFP-cDNA-2 (red and purple, respectively) that is consistent with theoretical mass increases of 2130 (2044 Da (cDNA-2)+86 Da (linker)) and 1983 Da (1897 Da (cDNA-2)+86 Da (linker)), respectively, for the functionalization of C148 mGFP with one strand of cDNA-2.

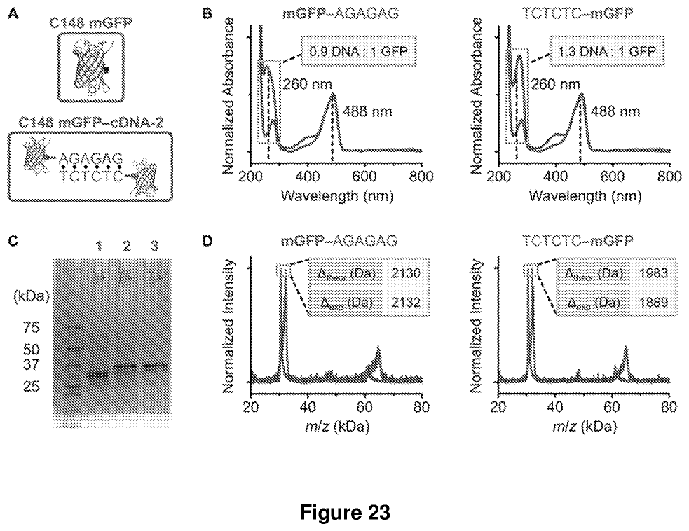

FIG. 24 shows the packing arrangement of the C148 mGFP-cDNA-2 crystal structure (6UHO). The packing arrangement of C148 mGFP (the two proteins in the asymmetric unit are teal and green with surface cysteines colored in red) in the C148 mGFP-cDNA-2 crystal structure.

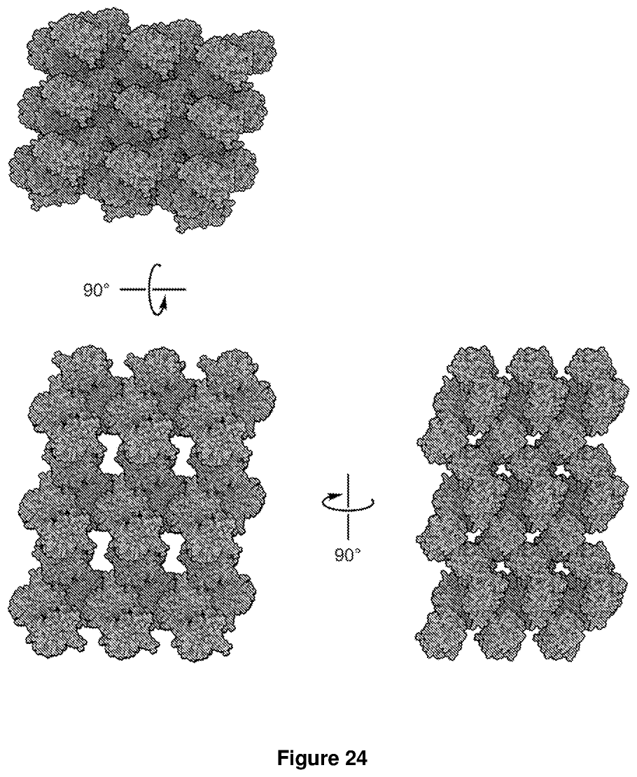

FIG. 25 depicts a structural comparison of crystals modified by different DNA interactions of equal length. Depicted is a comparison of C148 mGFP-scDNA-1, C148 mGFP-cDNA-1, and C148 mGFP-cDNA-2 crystal structures The C148 mGFP mutant was modified with three distinct DNA interactions of the same length and crystallized. The asymmetric unit for the structures of C148 mGFP-scDNA-1 (blue, 6UHL), C148 mGFP-cDNA-1 (red, 6UHN), and C148 mGFP-cDNA-2 (green, 6UHO) are overlaid. The root-mean-square deviations of all atoms between pairs of these structures are less than 0.2 Å, indicating that the structures are nearly equivalent.

FIG. 26 shows a characterization of C148 mGFP-ncDNA-1 conjugates. (A) Schematic of C148 mGFP (green) with the surface cysteine location marked in blue and schematic of C148 mGFP-ncDNA-1 (orange) depicting the DNA interaction between C148 mGFP-ncDNA-1 conjugates. (B) A UV-vis absorption spectrum that is normalized to the C148 mGFP (green) and C148 mGFP-ncDNA-1 (orange) chromophore absorbances at 488 nm. The increase in absorbance at 260 nm in C148 mGFP-ncDNA-1 relative to C148 mGFP corresponds to the presence of 1.1 ncDNA-1 per C148 mGFP in solution. (C) SDS PAGE analysis shows a mass increase from C148 mGFP (lane 1, green) to C148 mGFP-ncDNA-1 (lane 2, blue) that corresponds to conjugation of a single ncDNA-1 to C148 mGFP. The single band (˜32 kDa) for C148 mGFP-ncDNA-1 indicates high purity. (D) Mass characterization using MALDI-MS shows a mass increase of 1967 Da from C148 mGFP (green) to C148 mGFP-ncDNA-1 (orange) that is consistent with a theoretical mass increase of 2028 Da (1942 Da (ncDNA-1)+86 Da (linker)) for the functionalization of C148 mGFP with one strand of ncDNA-1.

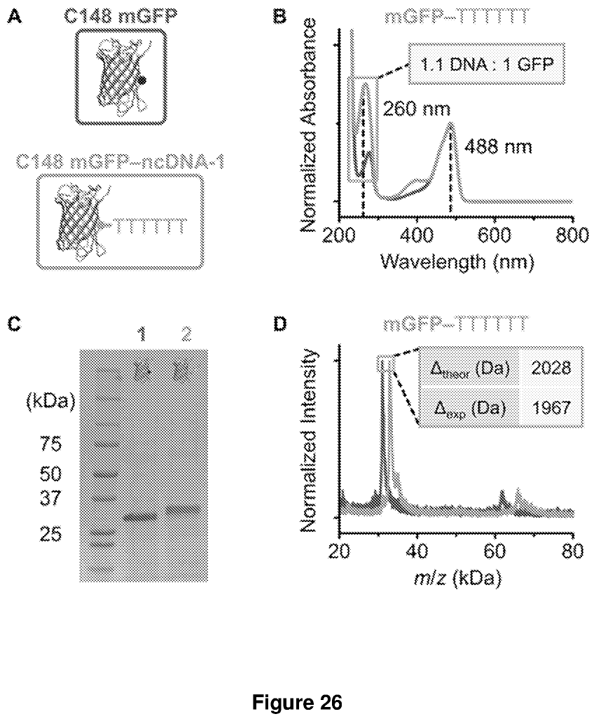

FIG. 27 shows the packing arrangement of the C148 mGFP-ncDNA-1 crystal structure (6UHP). The packing arrangement of C148 mGFP (the two proteins in the asymmetric unit are teal and green with surface cysteines colored in red) in the C148 mGFP-ncDNA-1 crystal structure.

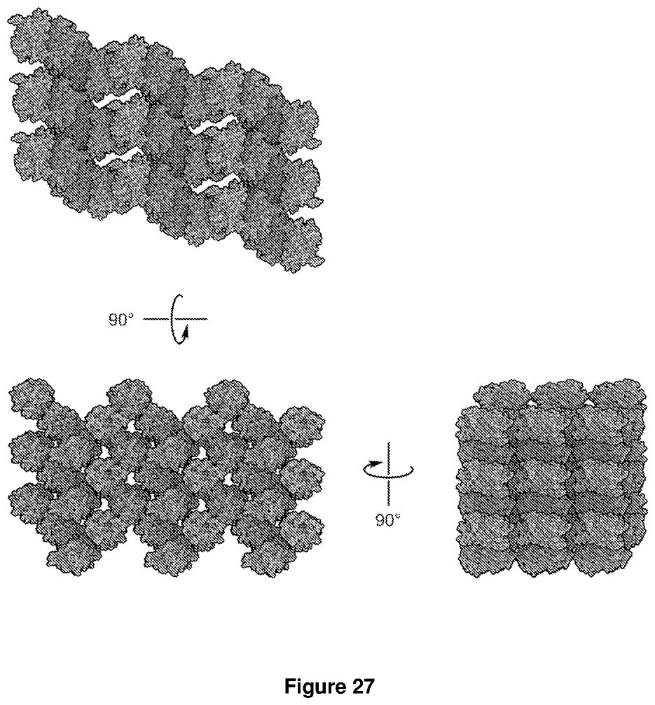

FIG. 28 shows the crystal structure of C148 mGFP-ncDNA-1 shows no free path between C148 residues (6UHP). Two asymmetric units from the C148 mGFP-ncDNA-1 crystal structure with mGFP proteins depicted in a space-filling manner (green). Each C148 (orange) orients towards distinct regions of solvent space with no free path in solvent space between C148 residues that would permit DNA hybridization. Protein-protein interactions (i and ii) block the path between C148 residues.

FIG. 29 shows the characterization of C148 mGFP-cDNA-3 conjugates. (A) Schematic of C148 mGFP (green) with the surface cysteine location marked in blue and schematic of C148 mGFP-cDNA-3 (red and purple for complementary DNA strands) depicting the DNA interaction between C148 mGFP-cDNA-3 conjugates. (B) UV-vis absorption spectra that are normalized to the C148 mGFP (green) and C148 mGFP-cDNA-3 (red or purple) chromophore absorbances at 488 nm. The increase in absorbance at 260 nm in C148 mGFP-cDNA-3 relative to C148 mGFP corresponds to the presence of 1.0 (red DNA design) and 1.1 (purple DNA design) cDNA-3 per C148 mGFP in solution. (C) SDS PAGE analysis shows a mass increase from C148 mGFP (lane 1, green) to C148 mGFP-cDNA-3 (lane 2, red and lane 3, purple) that corresponds to conjugation C148 mGFP one cDNA-3 for each complementary DNA strand. The primary band for each C148 mGFP-cDNA-3 (˜33 kDa) corresponds to C148 mGFP functionalized to a single cDNA-3 strand. Weak secondary bands at ˜30 and ˜60 kDa correspond to small impurities of C148 mGFP in the thiol and disulfide forms, respectively. (D) Mass characterization using MALDI-MS show mass increases of 3141 and 2789 Da from C148 mGFP (green) to each C148 mGFP-cDNA-3 (red and purple, respectively) that is consistent with theoretical mass increases of 3086 (3000 Da (cDNA-3)+86 Da (linker)) and 2881 Da (2795 Da (cDNA-3)+86 Da (linker)), respectively, for the functionalization of C148 mGFP with one strand of cDNA-3.

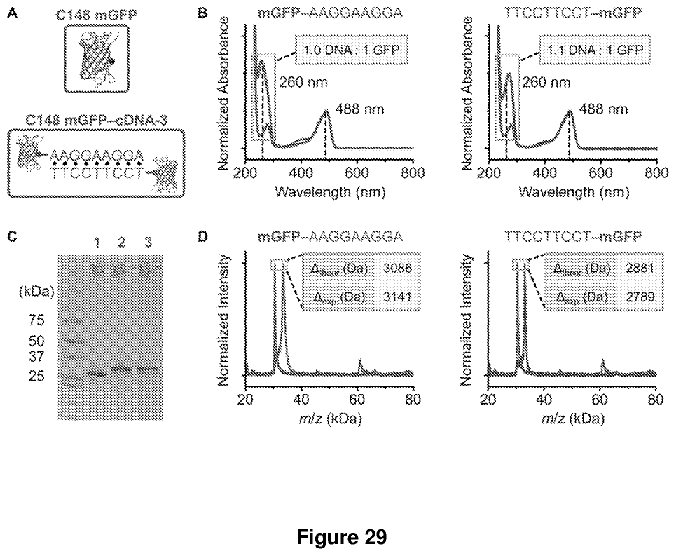

FIG. 30 shows the packing arrangement of the C148 mGFP-cDNA-3 crystal structure (6UHQ). The packing arrangement of C148 mGFP (colored in teal with surface cysteines colored in red) in the C148 mGFP-cDNA-3 crystal structure.

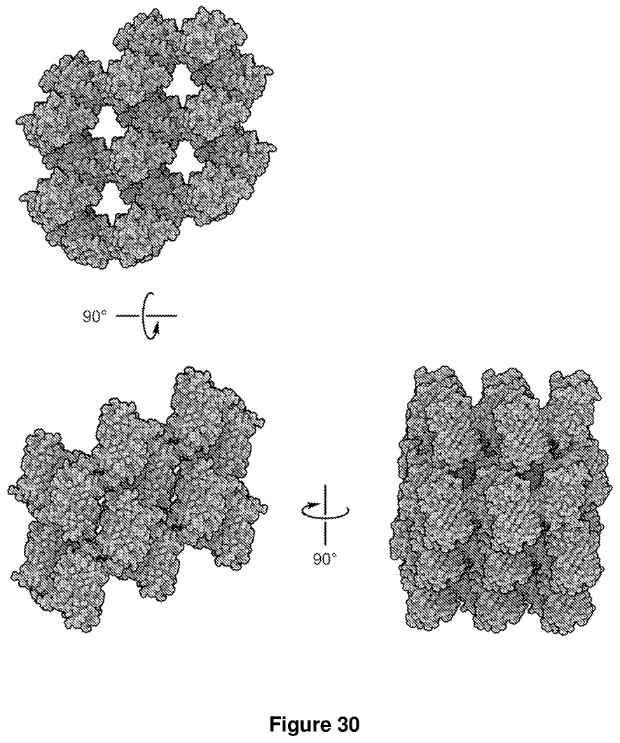

FIG. 31 shows the characterization of C148 mGFP-cDNA-4 conjugates. (A) Schematic of C148 mGFP (green) with the surface cysteine location marked in blue and schematic of C148 mGFP-cDNA-4 (red and purple for complementary DNA strands) depicting the DNA interaction between C148 mGFP-cDNA-4 conjugates. (B) UV-vis absorption spectra that are normalized to the C148 mGFP (green) and C148 mGFP-cDNA-4 (red or purple) chromophore absorbances at 488 nm. The increase in absorbance at 260 nm in C148 mGFP-cDNA-4 relative to C148 mGFP corresponds to the presence of 1.0 (red DNA design) and 1.1 (purple DNA design) cDNA-4 per C148 mGFP in solution. (C) SDS PAGE analysis shows a mass increase from C148 mGFP (lane 1, green) to C148 mGFP-cDNA-4 (lane 2, red and lane 3, purple) that corresponds to conjugation C148 mGFP one cDNA-4 for each complementary DNA strand. The primary band for each C148 mGFP-cDNA-4 (˜34 kDa) corresponds to C148 mGFP functionalized to a single cDNA-4 strand. A weak secondary band at ˜30 kDa corresponds to a small impurity of C148 mGFP in the thiol form. (D) Mass characterization using MALDI-MS show mass increases of 3887 and 3603 Da from C148 mGFP (green) to each C148 mGFP-cDNA-4 (red and purple, respectively) that is consistent with theoretical mass increases of 4058 (3972 Da (cDNA-4)+86 Da (linker)) and 3763 Da (3677 Da (cDNA-4)+86 Da (linker)), respectively, for the functionalization of C148 mGFP with one strand of cDNA-4.

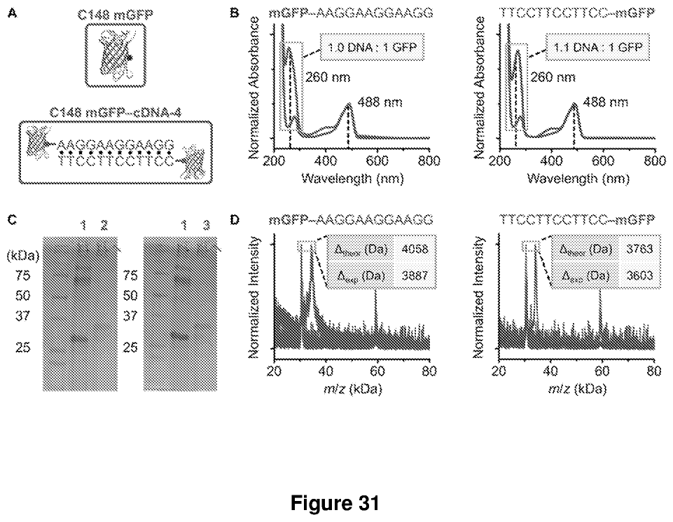

FIG. 32 shows the characterization of C148 mGFP-cDNA-5 conjugates. (A) Schematic of C148 mGFP (green) with the surface cysteine location marked in blue and schematic of C148 mGFP-cDNA-5 (red and purple for complementary DNA strands) depicting the DNA interaction between C148 mGFP-cDNA-5 conjugates. (B) UV-vis absorption spectra that are normalized to the C148 mGFP (green) and C148 mGFP-cDNA-5 (red or purple) chromophore absorbances at 488 nm. The increase in absorbance at 260 nm in C148 mGFP-cDNA-5 relative to C148 mGFP corresponds to the presence of 1.1 (red DNA design) and 1.0 (purple DNA design) cDNA-5 per C148 mGFP in solution. (C) SDS PAGE analysis shows a mass increase from C148 mGFP (lane 1, green) to C148 mGFP-cDNA-5 (lane 2, red and lane 3, purple) that corresponds to conjugation C148 mGFP one cDNA-5 for each complementary DNA strand. The primary band for each C148 mGFP-cDNA-5 (˜35 kDa) corresponds to C148 mGFP functionalized to a single cDNA-5 strand and the single band for each C148 mGFP-cDNA-5 indicates high purity.

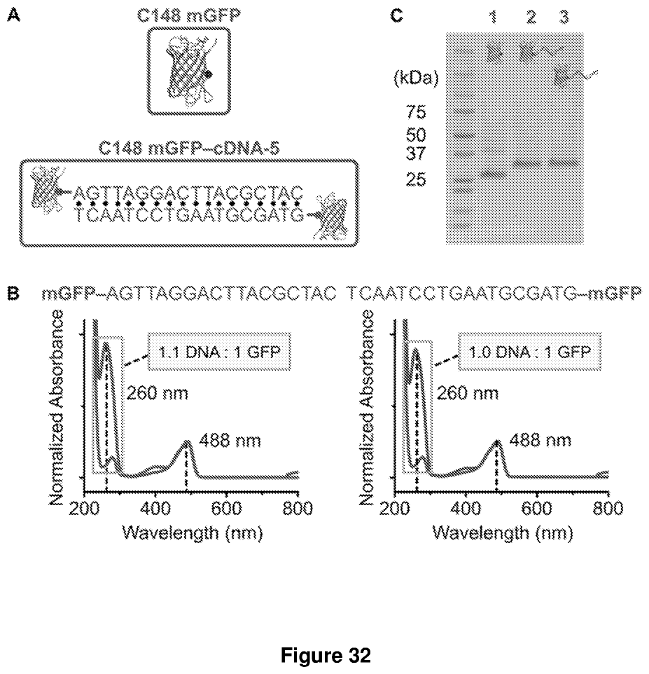

FIG. 33 shows the characterization of C148 mGFP-ncDNA-2 conjugates. (A) Schematic of C148 mGFP (green) with the surface cysteine location marked in blue and schematic of C148 mGFP-ncDNA-2 (orange) depicting the DNA interaction between C148 mGFP-ncDNA-2 conjugates. (B) A UV-vis absorption spectrum that is normalized to the C148 mGFP (green) and C148 mGFP-ncDNA-2 (orange) chromophore absorbances at 488 nm. The increase in absorbance at 260 nm in C148 mGFP-ncDNA-2 relative to C148 mGFP corresponds to the presence of 1.5 ncDNA-2 per C148 mGFP in solution. The ratio of ncDNA-2 to C148 mGFP is high, because some of the chromophores in C148 mGFP-ncDNA-2 were protonated as indicated by the absorbance at 395 nm. (C) SDS PAGE analysis shows a mass increase from C148 mGFP (lane 1, green) to C148 mGFP-ncDNA-2 (lane 2, orange) that corresponds to conjugation of a single ncDNA-2 to C148 mGFP. The primary band for each C148 mGFP-ncDNA-2 (˜35 kDa) corresponds to C148 mGFP functionalized to a single ncDNA-2 strand. A weak secondary band at ˜30 corresponds to a small impurity of C148 mGFP in the thiol form. (D) Mass characterization using MALDI-MS shows a mass increase of 4510 Da from C148 mGFP (green) to C148 mGFP-ncDNA-2 (orange) that is consistent with a theoretical mass increase of 2941 Da (2855 Da (ncDNA-2)+86 Da (linker)) for the functionalization of C148 mGFP with one strand of ncDNA-2.

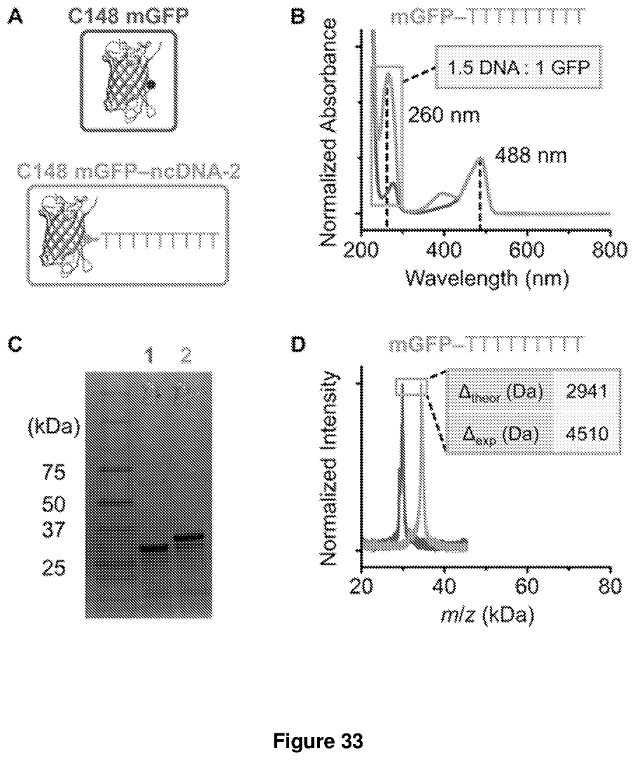

FIG. 34 shows the characterization of C176 mGFP-scDNA-1 conjugates. (A) Schematic of C176 mGFP (green) with the surface cysteine location marked in blue and schematic of C176 mGFP-scDNA-1 (blue) depicting the DNA interaction between C176 mGFP-scDNA-1 conjugates. (B) A UV-vis absorption spectrum that is normalized to the C176 mGFP (green) and C176 mGFP-scDNA-1 (blue) chromophore absorbances at 488 nm. The increase in absorbance at 260 nm in C176 mGFP-scDNA-1 relative to C176 mGFP corresponds to the presence of 2.0 scDNA-1 per C176 mGFP in solution. The ratio of scDNA-1 to C176 mGFP is high, because some of the chromophores in C176 mGFP-scDNA-1 were protonated as indicated by the absorbance at 395 nm. (C) SDS PAGE analysis shows a mass increase from C176 mGFP (lane 1, green) to C176 mGFP-scDNA-1 (lane 2, blue) that corresponds to conjugation of a single scDNA-1 to C176 mGFP. The primary band for each C176 mGFP-scDNA-1 (˜32 kDa) corresponds to C176 mGFP functionalized to a single scDNA-1 strand. Weak secondary bands at ˜30 and ˜60 kDa correspond to small impurities of C176 mGFP in the thiol and disulfide forms, respectively. (D) Mass characterization using MALDI-MS shows a mass increase of 1990 Da from C176 mGFP (green) to C176 mGFP-scDNA-1 (blue) that is consistent with a theoretical mass increase of 2016 Da (1930 Da (scDNA-1)+86 Da (linker)) for the functionalization of C176 mGFP with one strand of scDNA-1.

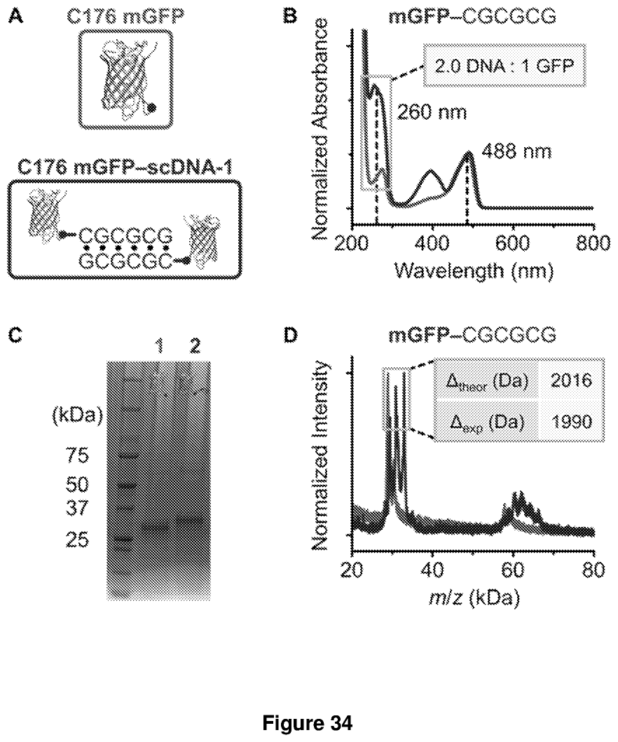

FIG. 35 shows the characterization of C191 mGFP-scDNA-1 conjugates. (A) Schematic of C191 mGFP (green) with the surface cysteine location marked in blue and schematic of C191 mGFP-scDNA-1 (blue) depicting the DNA interaction between C191 mGFP-scDNA-1 conjugates. (B) A UV-vis absorption spectrum that is normalized to the C191 mGFP (green) and C191 mGFP-scDNA-1 (blue) chromophore absorbances at 488 nm. The increase in absorbance at 260 nm in C191 mGFP-scDNA-1 relative to C191 mGFP corresponds to the presence of 0.8 scDNA-1 per C191 mGFP in solution. (C) SDS PAGE analysis shows a mass increase from C191 mGFP (lane 1, green) to C191 mGFP-scDNA-1 (lane 2, blue) that corresponds to conjugation of a single scDNA-1 to C191 mGFP. The primary band for each C191 mGFP-scDNA-1 (˜32 kDa) corresponds to C191 mGFP functionalized to a single scDNA-1 strand. Weak secondary bands at ˜30 and ˜60 kDa correspond to small impurities of C191 mGFP in the thiol and disulfide forms, respectively. (D) Mass characterization using MALDI-MS shows a mass increase of 1990 Da from C191 mGFP (green) to C191 mGFP-scDNA-1 (blue) that is consistent with a theoretical mass increase of 2016 Da (1930 Da (scDNA-1)+86 Da (linker)) for the functionalization of C191 mGFP with one strand of scDNA-1.

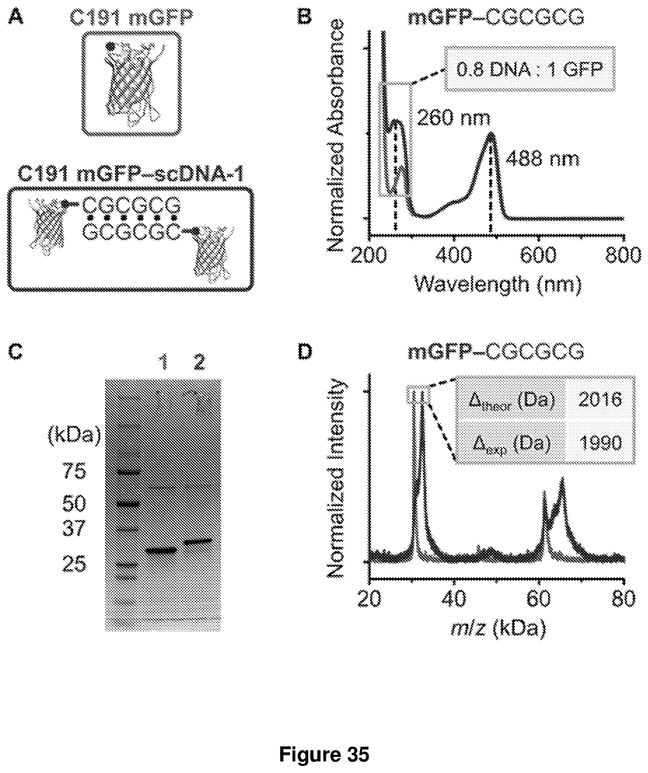

FIG. 36 shows the characterization of C148 mGFP-scDNA-2 conjugates. (A) Schematic of C148 mGFP (green) with the surface cysteine location marked in blue and schematic of C148 mGFP-scDNA-2 (blue) depicting the DNA interaction between C148 mGFP-scDNA-2 conjugates. (B) A UV-vis absorption spectrum that is normalized to the C148 mGFP (green) and C148 mGFP-scDNA-2 (blue) chromophore absorbances at 488 nm. The increase in absorbance at 260 nm in C148 mGFP-scDNA-2 relative to C148 mGFP corresponds to the presence of 1.3 scDNA-2 per C148 mGFP in solution. The ratio of scDNA-2 to C148 mGFP is high, because some of the chromophores in C148 mGFP-scDNA-2 were protonated as indicated by the absorbance at 395 nm. (C) SDS PAGE analysis shows a mass increase from C148 mGFP (lane 1, green) to C148 mGFP-scDNA-2 (lane 2, blue) that corresponds to conjugation of a single scDNA-2 to C148 mGFP. The primary band for each C148 mGFP-scDNA-2 (˜33 kDa) corresponds to C148 mGFP functionalized to a single scDNA-2 strand. Secondary bands at ˜30 and ˜60 kDa correspond to impurities of C148 mGFP in the thiol and disulfide forms, respectively. (D) Mass characterization using MALDI-MS shows a mass increase of 2559 Da from C148 mGFP (green) to C148 mGFP-scDNA-2 (blue) that is consistent with a theoretical mass increase of 2595 Da (2509 Da (scDNA-2)+86 Da (linker)) for the functionalization of C148 mGFP with one strand of scDNA-2.

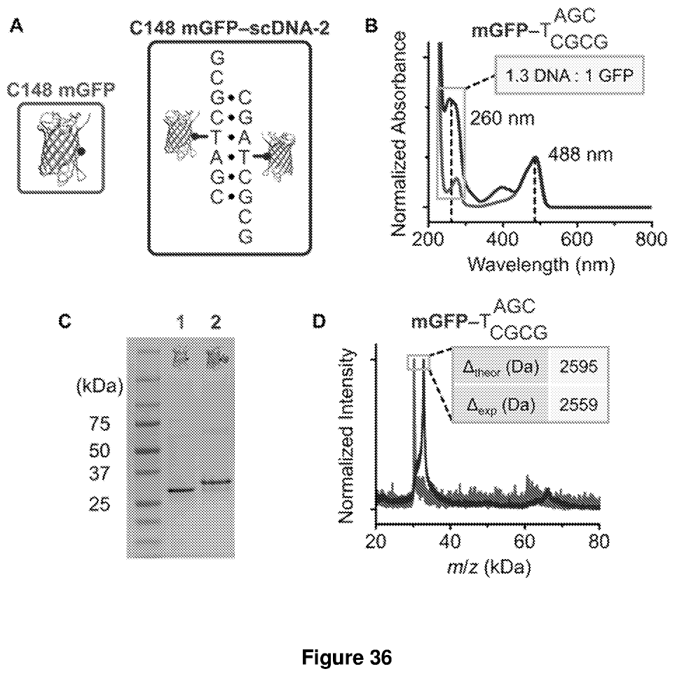

FIG. 37 shows the packing arrangement of the C148 mGFP-scDNA-2 crystal structure (6UHR). The packing arrangement of C148 mGFP (the two proteins in the asymmetric unit are teal and green with surface cysteines colored in red) in the C148 mGFP-scDNA-2 crystal structure.

FIG. 38 depicts design and parameter scope of mGFP-DNA conjugates that were studied. (A) Schematic of the DNA interaction between mGFP-DNA conjugates with dimensions for the mGFP, the DNA, and the mGFP-DNA linkage. (B) The design parameters explored included DNA sequence, DNA length, amino acid attachment position, and DNA base attachment position. DNA sequence was varied between self-complementary (scDNA), complementary (cDNA), and non-complementary (ncDNA), (upper left). DNA length was varied between 6 and 18 base pairs (upper right). DNA attachment positions were on the side (residue 148) or edge (residue 176 or 191) of the mGFPβ-barrel (lower left). The sites within the DNA for attachment to the proteins were either internal or external (lower right).

FIG. 39 depicts novel mGFP-DNA Single Crystal Structures. (A) A model of C148 mGFP (top). Four asymmetric units of the C148 mGFP crystal structure (6UHJ) in the space group P212121 (bottom), which is equivalent to previously reported GFP crystal structures (C148 residues represented in blue) [Arpino, J. A. J., Rizkallah, P. J., and Jones, D. D. (2012). Crystal Structure of Enhanced Green Fluorescent Protein to 1.35 Å Resolution Reveals Alternative Conformations for Glu222. PLoS One 7, e47132]. Proteins pack densely in this structure, and C148 is involved in an inter-protein interaction. (B) A model of the C148 mGFP-scDNA-1 design (top). Two asymmetric units of the C148 mGFP-scDNA-1 crystal structure (6UHL) in the space group P21 (bottom). aC148 mGFP-cDNA-1 and C148 mGFP-cDNA-2 crystallize into nearly identical structures (6UHN and 6UHO, see FIG. 25. In these structures, the DNA does not order past the disulfide mGFP-DNA attachment (inset). Pairs of C148 (blue) orient towards distinct regions of solvent space with a C148-C148 distance of 37±4 Å that is within the theoretical distance for DNA hybridization (27-64 Å). (C) A model of the C148 mGFP-ncDNA-1 design (top). Two asymmetric units from the C148 mGFP-ncDNA-1 crystal structure (6UHP) in the space group P21 (bottom), where each C148 (orange) orients towards distinct regions of solvent space with no free path between C148 residues that would permit DNA hybridization (see FIG. 28).

FIG. 40 shows confocal microscopy evidence for DNA in C148 mGFP-(s)cDNA and C148 mGFP-ncDNA crystals. Confocal microscopy images of (A) C148 mGFP, (B) C148 mGFP-ncDNA-1, and (C) C148 mGFP-cDNA-1 crystals after soaking the crystals for 30 minutes in the intercalating dye, TOTO-3. The images are in bright field (left), a green channel (middle, 485 nm excitation and 500-550 nm emission filter), and a far-red channel (right, 640 nm excitation and 663-738 nm emission filter). (D) The ratio of green to far-red fluorescence signals were compared across multiple crystals. The lower signal ratio in C148 mGFP-ncDNA-1 and C148 mGFP-cDNA-1 crystals compared to C148 crystals indicates the presence of DNA in C148 mGFP-ncDNA-1 and C148 mGFP-cDNA-1 crystals.

FIG. 41 shows that DNA design influences mGFP-DNA packing. (A) A model of the C148 mGFP-cDNA-3 design (top). Four asymmetric units of the C148 mGFP-cDNA-4 crystal structure (6UHQ) in the space group C2 (bottom). Increasing DNA duplex length by 3 bp led to this new structure. Pairs of C148 (red and purple) orient towards distinct regions of solvent space with a C148-C148 distance of 41±6 Å that is within the theoretical distance for DNA hybridization (37-75 Å). (B) A model of the C148 mGFP—scDNA-2 design (top). Two asymmetric units of the C148 mGFP—scDNA-2 crystal structure (6UHR) in the space group P212121 (bottom). Changing the location of mGFP-DNA attachment position led to this new structure. Pairs of C148 (blue) orient towards distinct regions of solvent space with a C148-C148 distance of 30±6 Å that is within the theoretical distance for DNA hybridization (8-45 Å).

### DETAILED DESCRIPTION

Advances in precise structural control of materials have led to dramatic improvements in technology and are the basis for modern approaches to materials chemistry. Bottom-up material synthesis allows for precise structural control, relying on programmable hierarchical ordering that leads to emergent materials properties. However, hierarchical ordering requires the ability to control orthogonal interactions over multiple lengths scales, which is a great challenge, especially in the context of proteins, where complex PPIs are responsible for organizing protein-based materials. For this reason, using proteins as building blocks for new materials lags far behind the advances made in other areas of materials science. The approach provided herein, to use the well-understood molecular interactions of DNA to control protein organization within protein crystals, enables unprecedented control over biomolecular architectures and the elucidation of protein crystal structure-activity relationships. The disclosure therefore provides, in various aspects, a novel approach to controlling biomolecular assembly to organize activity of biomolecules.

It is disclosed herein that modifying proteins with single stranded oligonucleotides influences crystallization, and when combined with protein-protein interactions, yields new crystal forms and atomic resolution structures.

The present disclosure discloses methods to induce crystallization of proteins using both protein-protein interactions (PP Is) and DNA hybridization interactions that are introduced onto the surface of a given protein through the covalent conjugation of a single (or multiple) oligonucleotide strand(s). The addition of this DNA tag imparts a protein with a handle that can be addressed to alter the crystallization outcome of the protein. The disclosure also provides compositions comprising the protein crystals formed by methods disclosed herein.

It is noted here that, as used in this specification and the appended claims, the singular forms “a,” “an,” and “the” include plural reference unless the context clearly dictates otherwise.

“About” and “approximately” shall generally mean an acceptable degree of error for the quantity measured given the nature or precision of the measurements. Exemplary degrees of error are within 20-25 percent (%), typically, within 10%, and more typically, within 5% of a given value or range of values.

All language such as “from,” “to,” “up to,” “at least,” “greater than,” “less than,” and the like include the number recited and refer to ranges which can subsequently be broken down into sub-ranges as discussed above.

A “conjugate” as used herein is a protein (which can be, e.g., a multimer or a monomer) or a fragment thereof that is attached to a polynucleotide.

As used herein a “fragment” of a protein is meant to refer to any portion of a protein smaller than the full-length protein or protein expression product.

Protein Crystal

A protein crystal comprises at least two conjugates, wherein a first conjugate comprises a first protein and a first polynucleotide and a second conjugate comprises a second protein and a second polynucleotide, wherein the first polynucleotide and the second polynucleotide are sufficiently complementary to hybridize to each other. In any of the aspects or embodiments of the disclosure, the first protein and the second protein associate with each other through a protein-protein interaction (PPI). The PPI, in various embodiments, is a hydrophobic bond, van der Waals forces, a salt bridge, a disulfide bond, an electrostatic interaction, hydrogen bonding, or a combination thereof. In some embodiments, the first protein and the second protein are the same. In further embodiments, the first protein and the second protein are different.

Proteins crystalized according to the methods described herein have both defined position and orientation in the unit cell. Formation of a protein crystal where protein orientation and position are defined using the methods described herein allows for the determination of the structure of these materials with angstrom resolution.

A conjugate comprises a protein or a fragment thereof that is attached to a polynucleotide. In various embodiments, a protein of the disclosure is attached to only one polynucleotide. In further embodiments, a protein of the disclosure is attached to 2, 3, 4, 5, 6, 7, 8, 9, 10, or more polynucleotides. A polynucleotide may be attached, in various embodiments, to the N-terminus, the C-terminus, or between the N-terminus and the C-terminus of a protein (via, e.g., a natural amino acid on the protein or an unnatural amino acid introduced into the protein via mutation).

Protein crystals of the disclosure are, in various embodiments, from about 250 nanometer (nm) to about 1 millimeter (mm), or from about 20 micrometers (pm) to about 500 pm in edge length. For example and without limitation, a preferred protein crystal size for synchrotron structure elucidation is about 20 μm to 100s of μm in edge length is preferred. For x-ray free electron laser structure elucidation, a preferred protein crystal size is from about 250nm to about 5 μm in edge length.

In various embodiments, a protein crystal of the disclosure has a pore size of from about 1 nanometer (nm) to about 100 nm in diameter. Porosity is varied in various embodiments by changing protein identity and oligonucleotide design (e.g., length, complementarity pattern).

Proteins

As used herein, protein is used interchangeably with “polypeptide” and refers to a polymer comprised of amino acid residues. A “monomer” as used herein refers to a contiguous polymer of amino acid residues. A “multimer” as used herein refers to at least two monomers that are associated with each other.

Proteins are understood in the art and include without limitation an antibody, an enzyme, a structural protein and a hormone. Thus, proteins contemplated by the disclosure include without limitation those having catalytic, signaling, therapeutic, or transport activity. In further embodiments, protein crystals are used for to determine the structure of proteins with unsolved structures. In some embodiments, a protein crystal produced by a method of the disclosure is an insulin crystal. In various embodiments, catalytic functionalities include biomedically related functions, such as replacing enzymes deficient in lysosomal storage disorders (a-galactosidase, β-glucosidase,β-cerebrosidase, aglucosidase-α, α-mannosidase,β-glucuronidase, α-glucosidase, β-hexosamininidase A, acid lipase, amongst others and variants of these enzymes), enzymes deficient in gastrointestinal disorders (lactase, lipases, amylases, or proteases), or enzymes involved in immunodeficiencies (adenosine deaminase), or include enzymes relevant for technological applications (hydrogenases, lipases, proteases, oxygenases, or laccases), which are in various embodiments used intra- or extracellularly. Signaling proteins include growth factors such as TNF-α or caspases. Human serum albumin is contemplated for use as a transport protein.

Proteins of the present disclosure may be either naturally occurring or non-naturally occurring.

Naturally Occurring Proteins

Naturally occurring proteins include without limitation biologically active proteins (including antibodies) that exist in nature or can be produced in a form that is found in nature by, for example, chemical synthesis or recombinant expression techniques. Naturally occurring proteins also include lipoproteins and post-translationally modified proteins, such as, for example and without limitation, glycosylated proteins.

Antibodies contemplated for use in the methods and compositions of the present disclosure include without limitation antibodies that recognize and associate with a target molecule either in vivo or in vitro.

Structural proteins contemplated by the disclosure include without limitation actin, tubulin, collagen, elastin, myosin, kinesin and dynein.

Non-Naturally Occurring Proteins

Non-naturally occurring proteins contemplated by the present disclosure include but are not limited to synthetic proteins, as well as fragments, analogs and variants of naturally occurring or non-naturally occurring proteins as defined herein. Non-naturally occurring proteins also include proteins or protein substances that have D-amino acids, modified, derivatized, or non-naturally occurring amino acids in the D- or L-configuration and/or peptidomimetic units as part of their structure. The term “peptide” typically refers to short polypeptides/proteins.

Non-naturally occurring proteins are prepared, for example, using an automated protein synthesizer or, alternatively, using recombinant expression techniques using a modified polynucleotide which encodes the desired protein.

Fusion proteins, including fusion proteins wherein one fusion component is a fragment or a mimetic, are also contemplated. A “mimetic” as used herein means a peptide or protein having a biological activity that is comparable to the protein of which it is a mimetic. By way of example, an endothelial growth factor mimetic is a peptide or protein that has a biological activity comparable to the native endothelial growth factor. The term further includes peptides or proteins that indirectly mimic the activity of a protein of interest, such as by potentiating the effects of the natural ligand of the protein of interest.

Proteins include antibodies along with fragments and derivatives thereof, including but not limited to Fab' fragments, F(ab)2 fragments, Fv fragments, Fc fragments, one or more complementarity determining regions (CDR) fragments, individual heavy chains, individual light chain, dimeric heavy and light chains (as opposed to heterotetrameric heavy and light chains found in an intact antibody, single chain antibodies (scAb), humanized antibodies (as well as antibodies modified in the manner of humanized antibodies but with the resulting antibody more closely resembling an antibody in a non-human species), chelating recombinant antibodies (CRABs), bispecific antibodies and multispecific antibodies, and other antibody derivative or fragments known in the art.

Polynucleotides

The terms “polynucleotide” and “oligonucleotide” are used interchangeably herein. Polynucleotides contemplated by the present disclosure include DNA, RNA, modified forms and combinations thereof as defined herein. Accordingly, in any of the aspects or embodiments of the disclosure, the protein crystal comprises DNA. In any of the aspects or embodiments of the disclosure, each polynucleotide that is part of a protein crystal is DNA. In any of the aspects or embodiments of the disclosure, each polynucleotide that is part of a protein crystal is RNA. In any of the aspects or embodiments of the disclosure, each polynucleotide that is part of a protein crystal is a modified polynucleotide. In some embodiments, the polynucleotides that are part of a protein crystal contain any combination of DNA, RNA, and/or modified polynucleotides. In any of the aspects or embodiments of the disclosure, the DNA is single-stranded. In some embodiments, the DNA is double stranded. In further aspects, the protein crystal comprises RNA, and in still further aspects the protein crystal comprises double stranded RNA. The term “RNA” includes duplexes of two separate strands, as well as single stranded structures. Single stranded RNA also includes RNA with secondary structure. In one aspect, RNA having a hairpin loop in contemplated.

The protein crystal comprises, in various embodiments, a first protein that is attached to a polynucleotide comprising a sequence that is sufficiently complementary to a polynucleotide that is attached to a second protein such that hybridization of the polynucleotide that is attached to the first protein and the polynucleotide that is attached to the second protein takes place. The polynucleotides are typically each single-stranded, but in various aspects one or more polynucleotides may be double stranded as long as the double stranded molecule also includes a single strand sequence that hybridizes to a single strand sequence of the second polynucleotide.

In some aspects, polynucleotides contain a spacer as described herein.

A “polynucleotide” is understood in the art to comprise individually polymerized nucleotide subunits. The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases adenine (A), guanine (G), cytosine (C), thymine (T) and uracil (U). Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and WO 97/12896, the disclosures of which are incorporated herein by reference. Modified nucleotides include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1 H-pyrimido[5,4-b][1,4]benzoxazin-2(3H)-one), phenothiazine cytidine (1 H-pyrimido[5 ,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

A polynucleotide of the disclosure, or a modified form thereof, is generally from about 3 nucleotides to about 50 nucleotides in length. In general, the length of the polynucleotide will depend on protein size and where in the nucleotide sequence the polynucleotide is attached to the protein. More specifically, a conjugate comprises a polynucleotide that is about 2 to about 40 nucleotides in length, about 2 to about 30 nucleotides in length, about 2 to about 20 nucleotides in length, about 2 to about 10 nucleotides in length, or about 2 to about 5 nucleotides in length, and all polynucleotides intermediate in length of the sizes specifically disclosed to the extent that the polynucleotide is able to achieve the desired result. Accordingly, polynucleotides of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides in length are contemplated. Specifically contemplated herein are polynucleotides that are 2 to 30 nucleotides, or 5 to 20 nucleotides, or 6 to 10 nucleotides in length.

Spacers

In certain aspects, protein crystals are contemplated which include those wherein a conjugate comprises a polynucleotide which further comprises a spacer.

“Spacer” as used herein means a moiety that serves to increase distance between the polynucleotide and the protein to which the polynucleotide is attached. In some embodiments, the spacer may be all or in part complementary to a second polynucleotide.

In some embodiments, the spacer when present is an organic moiety. In further embodiments, the spacer is a polymer, including but not limited to a water-soluble polymer, a nucleic acid, a protein, an oligosaccharide, a carbohydrate, a lipid, or combinations thereof.

The length of a spacer, in various embodiments, is or is equivalent to at least about 5 nucleotides, at least about 10 nucleotides, 10-30 nucleotides, 10-40 nucleotides, 10-50 nucleotides, 10-60 nucleotides, or even greater than 60 nucleotides. The spacers should not have sequences complementary to each other or to that of the polynucleotides. In certain aspects, the bases of a polynucleotide spacer are all adenines, all thymines, all cytidines, all guanines, all uracils, or all some other modified base. In some embodiments, a spacer does not contain nucleotides, and in such embodiments the spacer length is equivalent to at least about 5 nucleotides, at least about 10 nucleotides, 10-30 nucleotides, 10-40 nucleotides, 10-50 nucleotides, 10-60 nucleotides, or even greater than 60 nucleotides.

Modified Polynucleotides

As discussed above, modified polynucleotides are contemplated for use in producing a protein crystal. In various aspects, a polynucleotide of the disclosure is completely modified or partially modified. Thus, in various aspects, one or more, or all, sugar and/or one or more or all internucleotide linkages of the nucleotide units in the polynucleotide are replaced with “non-naturally occurring” groups.

In one aspect, the disclosure contemplates use of a peptide nucleic acid (PNA). In PNA compounds, the sugar-backbone of a polynucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

Other linkages between nucleotides and unnatural nucleotides contemplated for the disclosed polynucleotides include those described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et. al., Current Opinion in Structural Biology 5:343-355 (1995) and Susan M. Freier and Karl-Heinz Altmann, Nucleic Acids Research, 25:4429-4443 (1997), the disclosures of which are incorporated herein by reference.

Specific examples of polynucleotides include those containing modified backbones or non-natural internucleoside linkages. Polynucleotides having modified backbones include those that retain a phosphorus atom in the backbone and those that do not have a phosphorus atom in the backbone. Modified polynucleotides that do not have a phosphorus atom in their internucleoside backbone are considered to be within the meaning of “polynucleotide.”

Modified polynucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are polynucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated.

Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified polynucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In still other embodiments, polynucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂-described in U.S. Pat. Nos. 5,489,677, and 5,602,240. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In various forms, the linkage between two successive monomers in the polynucleotide consists of 2 to 4, desirably 3, groups/atoms selected from —CH2—, —O, —S—, —NRH—, >O═O, >C═NRH, >C═S, —Si(R″)₂—, —SO—, -S(O)₂—, —P(O)₂—, —PO(BH₃)—, —P(OS)—, —P(S)₂—, —PO(R″)—, —PO(OCH₃)—, and —PO(NHRH)—, where RH is selected from hydrogen and C1-4-alkyl, and R″ is selected from C1-6-alkyl and phenyl. Illustrative examples of such linkages are —CH₂—CH₂—CH₂-, —CH₂—CO—CH₂—, —CH₂—CHOH—CH₂-, —O—CH₂—O—, —O—CH₂—CH₂-, —O—CH₂—CH═(including R₅ when used as a linkage to a succeeding monomer), —CH₂—CH₂—O—, —NRH—CH₂—CH₂-, —CH₂—CH₂-NRH—, —CH₂—NRH—CH₂—, —O—CH₂—CH₂-NRH—, —NRH—CO—O—, —NRH—CO—NRH—, —NRH—CS—NRH—, —NRH—C(═NRH)—NRH—, —NRH—CO—CH₂—NRH—O—OC—O—, —O—OC—CH₂—O—, —O—CH₂—OC—O—, —CH₂—CO—NRH—, —O—OC—NRH—, —NRH—CO—CH₂—, —O—OH₂—OC—NRH—, —O—CH₂—CH₂—NRH—, —CH═N—O—, —CH₂—NRH—O—, —CH₂—O—N═(including R₅ when used as a linkage to a succeeding monomer), —CH₂—O—NRH—, —CO—NRH—CH₂—, —CH₂—NRH—O—, —CH₂—NRH—CO—, —O—NRH—CH₂—, —O—NRH, —O—CH₂—S—, —S—CH₂—O—, —CH₂—CH₂—S—, —O—CH₂—CH₂—S—, —S—CH₂—CH═(including R₅ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NRH—, —NRH—S(O)₂—CH₂—; —O—S(O)₂—CH₂—, —O—P(O)₂—O—, —O—P(OS)—O—, —O—P(S)₂—O—, —S—P(O)₂—O—, —S—P(OS)—O—, —S—P(S)₂—O—, —O—P(O)₂—S—, —O—P(OS)—S—, —O—P(S)₂—S—, —S—P(O)₂—S—, —S—P(OS)—S—, —S—P(S)₂—S—, —O—PO(R″)—O—, —O—PO(OCH₃)—O—, —O—PO(OCH₂CH₃)—O—, —O—PO(OCH₂CH₂S-R)—O—, —O—PO(BH₃)—O—, —O—PO(NHRN)—O—, —O—P(O)₂—NRH H—, —NRH—P(O)₂—O—, —O—P(O,NRH)—O—, —CH₂—P(O)₂—O—, —O—P(O)₂—CH₂—, and —O—Si(R″)₂—O—; among which —CH₂—CO—NRH—, —CH₂-NRH—O—, —S—CH₂—O—, —O—P(O)₂—O—O—P(—O,S)—O—, —O—P(S)₂—O—, —NRH P(O)₂—O—, —O—P(O,NRH)—O—, —O—PO(R″)—O—, —O—PO(CH₃)—O—, and —O—PO(NHRN)—O—, where RH is selected form hydrogen and C₁₋₄-alkyl, and R″ is selected from C₁₋₆-alkyl and phenyl, are contemplated. Further illustrative examples are given in Mesmaeker et. al., 1995, Current Opinion in Structural Biology, 5: 343-355 and Susan M. Freier and Karl-Heinz Altmann, 1997, Nucleic Acids Research, vol 25: pp 4429-4443.

Still other modified forms of polynucleotides are described in detail in U.S. Patent Publication No. 20040219565, the disclosure of which is incorporated by reference herein in its entirety.

Modified polynucleotides may also contain one or more substituted sugar moieties. In certain aspects, polynucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C1 to C10 alkyl or C2 to C10 alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂),ONH₂, and O(CH₂),ON[(CH₂),CH₃]₂, where n and m are from 1 to about 10. Other polynucleotides comprise one of the following at the 2′ position: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a polynucleotide, or a group for improving the pharmacodynamic properties of a polynucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin et al., 1995, HeIv. Chim. Acta, 78: 486-504) i.e., an alkoxyalkoxy group. Other modifications include 2′-dimethylaminooxyethoxy, i.e., a O(CH₂)20N(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′—CH₂—CH═CH₂), 2′-0-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the polynucleotide, for example, at the 3′ position of the sugar on the 3′ terminal nucleotide or in 2′-5′ linked polynucleotides and the 5′ position of 5′ terminal nucleotide. Polynucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects a methylene (—CH₂—)n group bridging the 2′ oxygen atom and the 4′ carbon atom wherein n is 1 or 2. LNAs and preparation thereof are described in WO 98/39352 and WO 99/14226, the disclosures of which are incorporated herein by reference.

Polynucleotide Complementarity

“Hybridization” means an interaction between two strands of nucleic acids by hydrogen bonds in accordance with the rules of Watson-Crick DNA complementarity, Hoogstein binding, or other sequence-specific binding known in the art. Hybridization can be performed under different stringency conditions known in the art. Under appropriate stringency conditions, hybridization can occur between two polynucleotides that are about 60% or above, about 70% or above, about 80% or above, about 90% or above, about 95% or above, about 96% or above, about 97% or above, about 98% or above, or about 99% or above complementary to each other.

In various aspects, the methods include use of polynucleotides that are 100% complementary to each other, i.e., a perfect match, while in other aspects, the polynucleotides are at least (meaning greater than or equal to) about 95% complementary to each other over the relevant length, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to each other over the relevant length. By relevant length is meant the length of a polynucleotide that hybridizes to another polynucleotide as disclosed herein. For example and without limitation, a polynucleotide strand having 21 nucleotide units can base pair with another polynucleotide of 21 nucleotide units, yet only 19 bases on each strand are complementary or sufficiently complementary, such that the “duplex” has 19 base pairs. The remaining bases may, for example, exist as 5′ and/or 3′ overhangs. Further, within the duplex, 100% complementarity is not required; substantial complementarity is allowable within a duplex. Sufficient complementarity refers, in various embodiments, to 75%, 80%, 85%, 90%, 95%, 99% or 100% complementarity.

Protein Crystal Synthesis

Crystallization of proteins into highly ordered single crystals enables, e.g., determination of protein structure as well as the synthesis of functional crystalline materials. Multiple factors influence crystal formation (e.g., protein-protein interactions, buffer conditions, temperature) but few can be rationally designed to program how proteins crystallize and influence the way in which they pack. In the present disclosure, some protein-protein interactions were replaced with highly programmable DNA interactions to drive crystallization of proteins into new structures.

The methods of the disclosure enable a way to influence the packing of proteins within single crystals. The orientation of proteins within the crystal can be influenced by the selection of the location on the protein where the polynucleotide is attached. Additionally, the distance between sections of the protein surface can be tuned by varying oligonucleotide length. Materials with designable protein orientation and distance have applications, for example, as catalytic materials, where it may be important to control how enzymatic active sites are arranged in a material.

The methods of the disclosure also provide a way to co-crystallize multiple proteins through the attachment of complementary polynucleotides to distinct proteins. These aspects have applications in, for example, in catalysis, where multiple enzymatic proteins can be co-crystallized to form a cascade catalytic material. In further embodiments, the methods also provide a mechanism for novel protein structure determination, where a novel protein modified with a polynucleotide can be directed to crystallize via the attachment of a complementary polynucleotide to a protein that readily crystallizes. That protein crystal can then be used for structure determination of the novel protein. In further embodiments, oligonucleotide (e.g., DNA) hybridization directs novel proteins to crystallize without the help of a protein that crystallizes readily. For example, the first protein and the second protein could both be the same protein or different novel proteins.

An advantage of the methods of the disclosure over other routes that link proteins together prior to crystallization is that distinct complementary pairs of polynucleotides can be designed and attached to proteins which provide the ability to couple numerous proteins together, and proteins in various structural orientations. In some embodiments, crystallization of proteins attached to multiple distinct polynucleotides enables additional influence over the packing of proteins within crystals or the co-crystallization more than two proteins.

Polynucleotide Attachment to a Protein

In any of the aspects or embodiments of the disclosure, polynucleotides are covalently attached to a surface-exposed amino acid of a protein, including the N- and C-terminal amino acids. In some embodiments, an amine-modified polynucleotide is attached to a surface-exposed cysteine using an amine-to-sulfyhydryl crosslinker. As disclosed herein, however, many other routes exist to attach a polynucleotide (e.g., DNA) to specific amino acids on proteins. Proteins that are modified with a polynucleotide strand are purified using methods known in the art (e.g., affinity and anion-exchange chromatography, size-exclusion chromatography). The attachment of a polynucleotide to a protein and the successful purification of protein-polynucleotide conjugates may be confirmed using, e.g., UV-vis spectroscopy, SDS polyacrylamide gel electrophoresis, and/or matrix-assisted laser desorption/ionization mass spectrometry.

A polynucleotide may be attached to any surface-exposed amino acids on a protein, including but not limited to the N- and C-termini. In various embodiments, proteins naturally have a single amino acid that can be targeted for attachment of a single oligonucleotide or proteins can be modified using molecular biology tools (mutagenesis, genetic code expansion, etc.) that can be targeted for the specific attachment of a single oligonucleotide. In some embodiments, smaller proteins require shorter polynucleotide lengths (e.g., 2-9 nucleotides) while larger proteins may require longer oligonucleotide lengths (e.g., 10-30 nucleotides).

A polynucleotide can be modified at a terminus with an alkyne moiety, e.g., a DBCO-type moiety for reaction with the azide of the protein surface. Polynucleotides may be attached to a protein through any means (e.g., covalent or non-covalent attachment). Regardless of the means by which the oligonucleotide is attached to the protein, attachment in various aspects is effected through a 5′ linkage, a 3′ linkage, some type of internal linkage, or any combination of these attachments. In some embodiments, the polynucleotide is covalently attached to a protein. In further embodiments, the polynucleotide is non-covalently attached to a protein.

The surface functional group of a protein can be attached to the polynucleotide using other attachment chemistries. For example and without limitation, a surface amine can be directed conjugated to a carboxylate or activated ester at a terminus of the polynucleotide, to form an amide bond. In some embodiments, the surface amino group is from a lysine (Lys) residue. A surface carboxylate can be conjugated to an amine on a terminus of the polynucleotide to form an amide bond. Alternatively, the surface carboxylate can be reacted with a diamine to form an amide bond at the surface carboxylate and an amine at the other terminus. This terminal amine can then be modified in a manner similar to that for a surface amine of the protein. A surface thiol can be conjugated with a thiol moiety on the polynucleotide to form a disulfide bond. Alternatively, the thiol can be conjugated with an activated ester on a terminus of a polynucleotide to form a thiocarboxylate. In some embodiments, a polynucleotide is attached to a protein via a triazole linkage formed from reaction of (a) an azide moiety attached to the surface amino group and (b) an alkyne functional group on the first polynucleotide. In further embodiments, a polynucleotide is attached to a protein via native chemical ligation, other amino acid functionalities such as tyrosine, methionine, and/or serine, or noncovalent peptide interactions (such as, without limitation, coiled-coil interactions and protein-ligand interactions).

Choosing Oligonucleotide Design and Position

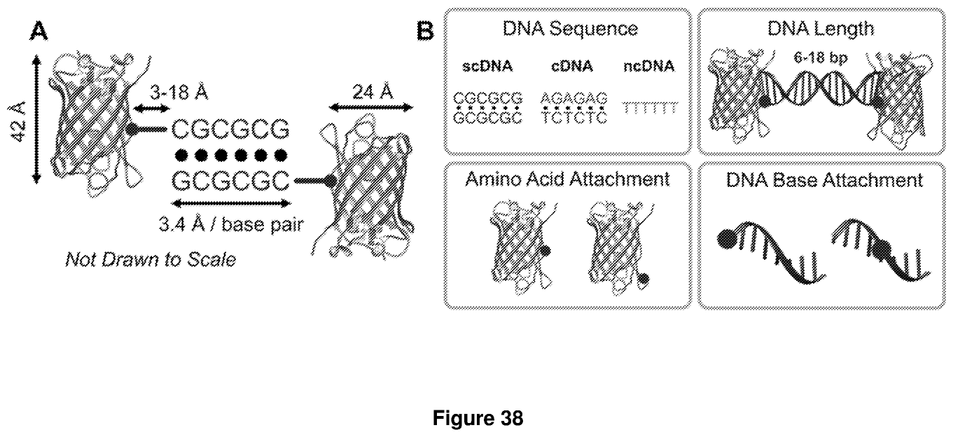

In various embodiments, and as exemplified herein, the sequence of a polynucleotide, the length of a polynucleotide, the amino acid position to which the polynucleotide was attached, and the polynucleotide base position to which the protein was attached was all varied (see FIG. 38). In some embodiments, the changes in polynucleotide structure lead to changes in how proteins pack relative to each other in single crystals, enabling the design of crystal architecture.

Polynucleotide length determines whether a conjugates crystallize and influences the protein packing within crystals that do form. As polynucleotide length increases, the amino acids attached to a polynucleotide become spaced farther apart. In some embodiments in which a crystal forms, a polynucleotide that is part of a conjugate is 9 nucleotides in length or less. In further embodiments in which a crystal forms, a polynucleotide that is part of a conjugate is or is about 30, 29, 28, 27, 26, 25, 24, 23, 22, 21, 20, 19, 18, 17, 16, 15, 14, 13, 12, 11, 10, 8, 7, 6, 5, 4, or 3 nucleotides in length or less. In further embodiments in which a crystal forms, a polynucleotide that is part of a conjugate is from about 2 to about 30, or from about 2 to about 20, or from about 2 to about 10, or from about 2 to about 5, or from about 5 to about 30, or from about 5 to about 20, or from about 5 to about 10, or from about 10 to about 30, or from about 10 to about 20, or from about 20 to about 30 nucleotides in length.

The polynucleotide base attachment position can be internal or external. In some embodiments, the attachment position influences the packing of proteins within crystals.

Protein Crystallization

In various aspects, the disclosure provides a method of producing a protein crystal comprising contacting a first conjugate comprising a first protein and a first polynucleotide with a second conjugate comprising a second protein and a second polynucleotide under conditions sufficient such that the first polynucleotide and the second polynucleotide hybridize to each other and the first protein and second protein associate via protein-protein interactions (PPI) to form the protein crystal. In some embodiments, a first conjugate associates with a second conjugate strictly through hybridization of a polynucleotide attached to the first conjugate with a polynucleotide attached to the second conjugate (i.e., no protein-protein interactions are involved in the association). In further embodiments, the contacting step further comprises contacting the first conjugate and/or the second conjugate with a third conjugate comprising a third protein and a third polynucleotide, wherein the third polynucleotide hybridizes to the first polynucleotide or the second polynucleotide, and the resulting protein crystal comprises the first protein, second protein, and third protein.

Crystallization is also dependent, in various embodiments, on the amino acid attachment position. In some embodiments, positions with lower flexibility can lead to crystal formation, while positions with higher flexibility do not crystallize.

Protein-polynucleotide conjugates are crystallized using methods that are used for crystallizing proteins, which are distinct from the methods for crystallization that are used in, e.g., Brodin et al. (Proc. Natl. Acad. Sci. U. S. A. 112, 4564-4569 (2015)).

In general, protein-polynucleotide conjugates are concentrated and mixed with a solution containing salt (one or more of calcium chloride, magnesium chloride, lithium sulfate, ammonium sulfate, sodium chloride, etc.), and a buffer (e.g., HEPES, MES, Tris). PEG or analogous polymers (MW 400 to 20,000, 0 — 50% w/v) may also be added. Protein-polynucleotide conjugates mixed with the foregoing solutions are then crystallized with vapor-diffusion. Protein-polynucleotide conjugates form highly ordered single crystals where protein structure can be determined. As described herein, protein-protein and/or polynucleotide-polynucleotide interactions contribute to such high ordering.

In any of the aspects or embodiments of the disclosure, the first conjugate and second conjugate interact through protein-protein interactions (PPIs) to form a crystal. In further embodiments, the first conjugate interacts through PPIs only with other copies of itself but still forms crystals with a second conjugate that interacts with the first conjugate only via polynucleotide hybridization.

Methods of Catalyzing a Reaction

Provided herein are methods of using the disclosed protein crystals as catalysts for a chemical reaction to transform one or more reagents to a product. The methods can comprise contacting the one or more reagents of the reaction with a protein crystal as disclosed herein such that contact of the reagent or reagents with the protein crystal results in the reaction being catalyzed to form a product of the reaction, wherein the protein or proteins in the crystal is an enzyme for the chemical reaction.

### EXAMPLES

Designed DNA interactions are investigated herein for their ability to modulate protein packing within single crystals of mutant green fluorescent proteins (mGFPs) functionalized with a single DNA strand (mGFP-DNA). DNA sequence, length, and protein-attachment position are probed for their effects on the formation and protein packing of mGFP-DNA crystals. Notably, when complementary mGFP-DNA conjugates are introduced to one another, crystals form with nearly identical packing parameters, regardless of sequence if the number of bases is equivalent. DNA complementarity is essential, as experiments with non-complementary sequences produce crystals with different protein arrangements. Importantly, the DNA length and its position of attachment on the protein markedly influence protein packing within the resulting single crystals. Above a threshold DNA duplex length (9 bp), no crystals form. This work showed how designed DNA interactions can be used to influence the growth and packing of x-ray diffraction quality protein single crystals and is thus an important step forward in protein crystal engineering.

### Example 1

### Synthesis and Characterization of Protein-DNA Conjugates

GFP was expressed in a bacterial expression system, and purified with Ni-NTA affinity and DEAE anion exchange. DNA was synthesized with solid-phase protocols with reagents purchased from Glen Research. The following sequences were used:

| Name | Sequence (5′ to 3′) |
|---|---|
| nc6mer | H₂N TTT TTT |
| sc6mer | H₂N CGC GCG |

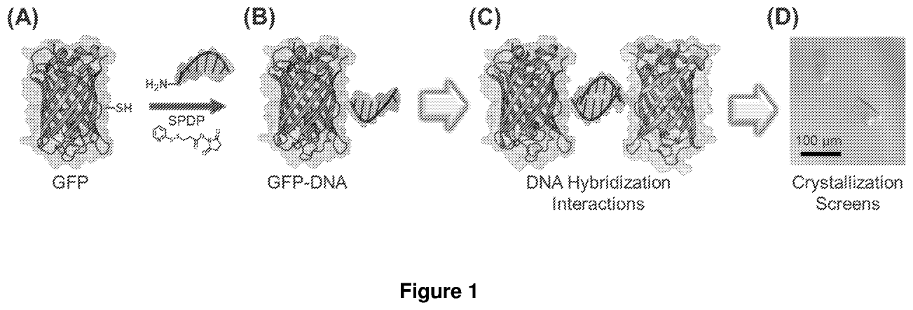

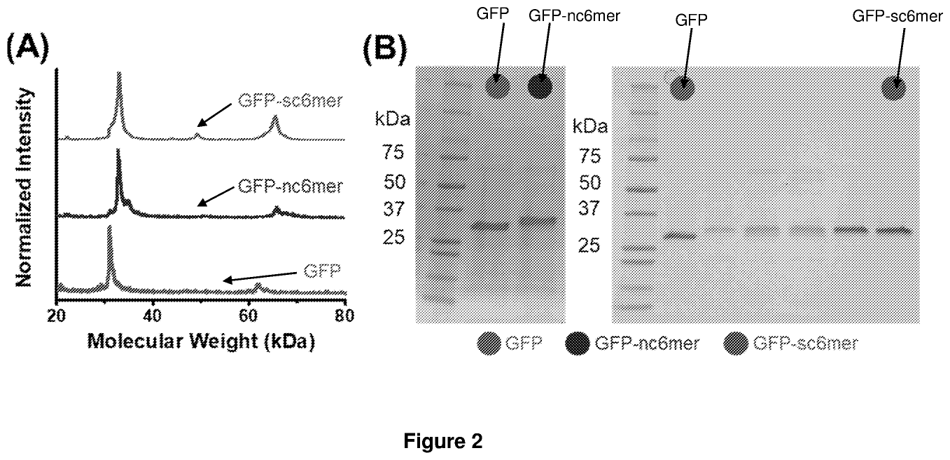

Pyridyl disulfide chemistry was used to conjugate DNA to the surface thiol on GFP. See FIG. 1. After amine-terminated DNA was reacted with succinimidyl 3-(2-pyridyldithio)propionate cross linker, the pyridyl disulfide terminated DNA was added in ten-fold excess to GFP. Ni-NTA affinity and DEAE anion exchange were used to purify GFP with a single DNA modification. MS MALDI and SDS-PAGE are evidence for successful conjugation and purification, with additional support from Uv-vis absorption spectra and size exclusion chromatography characterization (FIG. 2).

Crystallization screens.

Protein-DNA conjugates buffer exchanged from 1xPBS to 10 mM Tris Buffer 137 mM NaCI and concentrated to 5 mg/mL. Art Robbins Instruments Crystal Gryphon or a TTP Labtech Mosquito Crystal robot were used for high throughput crystal screens with Qiagen reagents. Qiagen crystal screens PEGs II, Classics II, JCSG+, and PACT were used to search for conditions in which the protein-DNA conjugates crystallized. GFP conjugated to a self-complimentary 6mer (GFP-sc6mer), a non-complimentary 6mer (GFP-nc6mer), and not conjugated to DNA (GFP) crystallized.

X-Ray Diffraction Experiments.

Obtained crystals were studied with synchrotron X-ray diffraction experiments at Argonne National Laboratory on the Advanced Photon Source with the Life Sciences Collaborative Access Team.

GFP crystallized to the same space group and unit cell as the majority of GFP structures in the Protein Data Bank. GFP-nc6mer crystallized with a novel unit cell. While DNA did not order, all cysteines for GFP-nc6mer pointed towards 26 Å pores and are spaced too far apart to be hybridized. GFP-sc6mer crystallized with a novel unit cell. While DNA did not order, pairs of cysteines pointed towards the same pore with a relevant distance between cysteines for the DNA to be hybridized.

### Example 2

### Rational Design of Protein Crystals of Arbitrary Composition and Structure, Directed by DNA Ligands

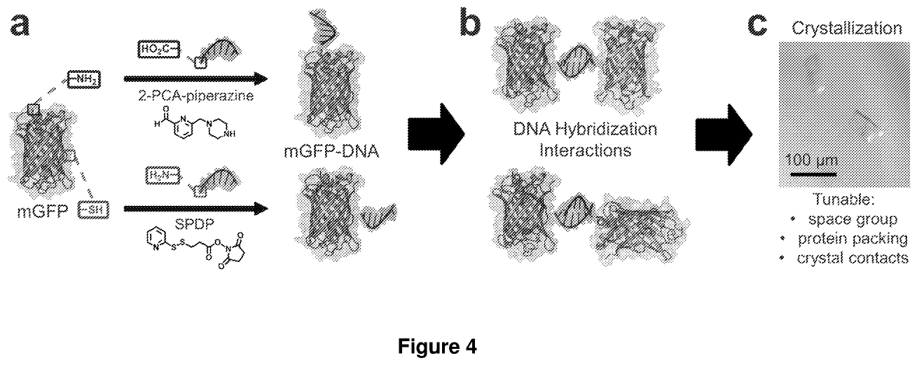

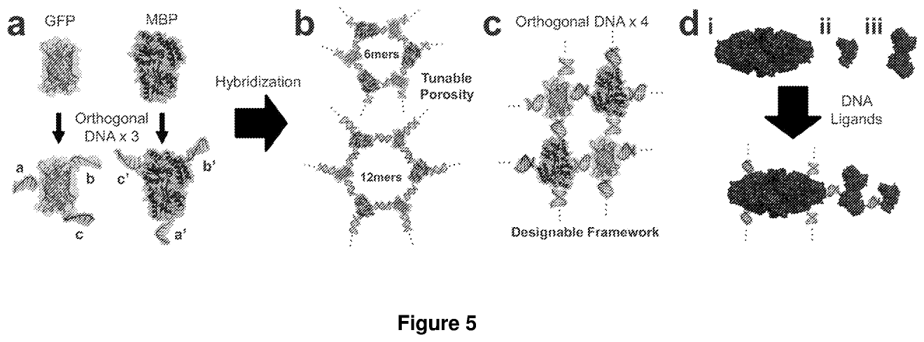

No methods currently exist to design the architecture of protein crystals. First, design rules are established for the crystallization of proteins using DNA ligands using a model system, (FIG. 4) and will investigate the programmable co-crystallization of two or more proteins with tunable crystal structure and porosity (FIG. 5). Designed co-crystallization of enzymes involved in an enzymatic pathway will lead to applications in cascade catalysis. Together, the proposed work will enable unprecedented control over protein organization within protein crystals, enabling synthesis of bio-materials that harness and combine proteins' specialized properties, from catalysis to fluorescence, with DNA's programmable assembly properties.

DNA Ligand Design Rules in Protein Crystal Engineering.

First it is established how the structural parameters of DNA ligands impact protein crystal structure and the design space within which protein crystals containing DNA interactions can be obtained. Crystallization experiments are conducted on a model system: a protein with well-established chemistry and structure, green fluorescent protein (GFP), modified with a single DNA ligand. DNA is bound to a single site at the GFP N-terminus²¹ or a unique surface residue introduced through mutagenesis (FIG. 4a).¹⁶ High-throughput crystallization screens and synchrotron X-ray diffraction experiments are performed. The crystal structure for this system elucidates contribution of PPIs on DNA ligand interactions to protein organization and represents the first hybrid protein-DNA conjugate to diffract to angstrom-level resolution, in contrast to other studies where polydisperse protein-DNA conjugates have failed to give orientationally ordered protein crystals¹⁷ or where crystals have not grown large enough to diffract to high resolution.²²

In elucidating a design space for DNA-assembled protein crystals, there are two key outputs that are considered: (1) how structural aspects of the DNA ligand and its attachment chemistry could either enable or inhibit protein crystallization, and (2) how DNA sequence can influence the structural outcome of these crystals. To this end, what range of flexibility, length and sequence of DNA is amenable to crystallization is determined. It is contemplated that by using a rigid protein-DNA linkage, resolution of conjugates in crystal structures are maximized. However, some linkage flexibility may be required, where flexibility permits a larger DNA sampling space to find an energetically favored hybridization orientation that minimizes steric repulsion. At short DNA lengths, Gibbs free energy of protein crystallization, ΔG_{cryst}, may be most negative where PPIs dominate crystallization and DNA ligands are entropically disordered, and therefore do not form a duplex in the protein crystal. In contrast, at longer DNA lengths, AGcr_{y}st may be most negative when DNA hybridizes. As DNA ligands approach the persistence length of double stranded DNA, high conformational variation may prevent crystallization. It is important to initially avoid sequences that form secondary structure, but with greater understanding of DNA ligand design rules, complexity is programmed into protein crystals. Hairpin or G-quadruplex DNA enables stimuli-responsive protein structures, while helical junctions or three-dimensional DNA shapes enables complex, multi-component protein architectures. Once this design space is established and understood, it is investigated how the complementarity, binding strength and placement of the designed DNA sequence (FIG. 4b) influences the packing of proteins in these crystals, tuning crystal contacts and space group (FIG. 4c). Attaching DNA may disrupt crystal contacts formed by native proteins and/or vary the relative ΔG_{cryst} of crystal contacts, thus modifying how protein-DNA conjugates crystallize. With correct DNA placement, DNA ligands enable the space group of the protein crystal to be programmed by enforcing specific symmetry. Together, mapping out these design rules for DNA ligands permits programmable and tunable DNA “bonds” that are independent of protein identity to be introduced into protein crystals.

Programmable Co-Crystallization of Multiple Proteins for Cascade Catalysis.

Most biological processes combine functions of two or more proteins, but to date, protein crystals comprised of multiple proteins with designed orientations and spacing have not been realized experimentally.²³⁻²⁵ Extending protein crystal engineering to multiple proteins is important to increase the application scope, for example, enabling cascade catalysis. Moreover, programmed protein orientation enhances catalytic synergy between proteins. Working towards such an application, there are two phases of study: (1) programmable co-crystallization of different model proteins to determine rules that dictate architectural control over multi-protein crystals and (2) application of these rules to co-crystallize relevant enzymes for a cascade catalysis reaction. In phase one, GFP and maltose binding protein (MBP) are used, another protein with well-known chemistry and structure. First, the crystallization of GFP and MBP is studied using a single DNA hybridization interaction, establishing whether DNA design rules for crystallization of a single protein extend to co-crystallization of multiple proteins. Next using multiple DNA ligands, porous protein frameworks are assembled and crystallized, structurally analogous to MOFs. For instance, conjugation of three orthogonal DNA sequences to GFP and MBP (to a N-termini, a surface cysteine and an unnatural amino acid) (FIG. 5a), may enable assembly of hexagonal frameworks with nanometer-scale pores, while maintaining controlled protein orientation and tunable porosity (FIG. 5b). Adding a 4th orthogonal DNA sequence results in rectangular frameworks (FIG. 5c). The independence of DNA ligand interactions and protein identity leads to rapid materials design of many frameworks with interchangeable nodes and linkers, similar to rapid synthesis of thousands of MOF structures.²⁶

In the second phase of this study, design parameters from the programmable co-crystallization model are applied to crystalize multiple enzymes along a cascade catalysis pathway. Protein crystals are an advantageous platform for heterogeneous enzyme catalysis, because they are often more thermally or chemically stable than free proteins.²⁷ However, no current methods exist for protein crystals to mediate consecutive reactions, control the orientation of multiple active sites, or design crystals with defined porosity to enable improved substrate diffusion. The organization and orientation of enzymes with DNA ligands leads to development of protein crystal cascade catalysts which overcome these challenges. Using DNA ligands, 6-galactosidase, hexokinase and glucose-6-phosphate dehydrogenase are co-crystallized, enzymes that catalyze a three-step conversion from lactose to an oxidized, phosphorylated glucose with specificity that is challenging to obtain synthetically (FIG. 5d).²⁸ It is contemplated that cascade catalysis rates depend upon crystal porosity, protein orientation and protein organization, parameters that are tunable with DNA-mediated protein crystallization.

Rational and programmable design of protein crystal materials that utilize and combine specialized protein properties opens a new field of advanced bio-materials.

### Example 3

Materials and Methods

Protein mutation, expression, and purification. A gene for C148 mGFP (Table 1) was cloned and transformed into One Shot® BL21(DE3) Chemically Competent E. coli (Thermo Fisher) in previous work [Hayes, O. G., McMillan, J. R., Lee, B., and Mirkin, C. A. (2018). DNA-Encoded Protein Janus Nanoparticles. J. Am. Chem. Soc. 140, 9269-9274]. Genes for C176 mGFP and C191 mGFP (Table 1), Integrated DNA Technologies) were cloned into the pET28 vector backbone using Gibson Assembly [Gibson, D. G., Young, L., Chuang, R.-Y., Venter, J. C., Hutchison Iii, C. A., and Smith, H.O. (2009). Enzymatic assembly of DNA molecules up to several hundred kilobases. Nat. Methods 6, 343-345]. The assembled plasmids were transformed into BL21(DE3) electrically competent cells (Thermo Fisher) with electroporation. After recovery in S.O.C. Medium (ThermoFisher) for 1 hour at 37° C. with 300 rpm shaking, cells were grown overnight on LB Agar plates with antibiotic (50 μg/mL kanamycin). Single colonies were selected and cultured in 8 mL of LB broth with antibiotic (50 μg/mL kanamycin) overnight at 37° C. with 200 rpm shaking. After cell growth, glycerol stocks of the cells were prepared and stored at −80° C. Plasmids were extracted from cells using the QlAprep Spin Minoprep Kit (Qiagen) and the correct plasmid sequences were confirmed using Sanger Sequencing (ACGT) [Sanger, F., Nicklen, S., and Coulson, A. R. (1977). DNA sequencing with chain-terminating inhibitors. Proc. Natl. Acad. Sci. U. S. A. 74, 5463-5467].

Cultures in 8 mL of LB broth with antibiotic (100 μg/mL ampicillin for C148 mGFP and 50 μg/mL kanamycin for C176 mGFP, and C191 mGFP) were inoculated using glycerol stocks and grown overnight at 37° C. with 200 rpm shaking. Next, these cultures were added to 1 L of 2× YTP broth with antibiotic (100 μg/mL ampicillin for C148 mGFP and 50 μg/mL kanamycin for C176 mGFP, and C191 mGFP) and grown at 37° C. with 200 rpm shaking until a cell OD at 600 nm of 0.6 (˜4 h). Cultures were induced (0.2% [w/w] L-arabinose for C148 mGFP and 1 mM IPTG for C176 mGFP, and C191 mGFP) and grown overnight at 17° C. with 200 rpm shaking. Cells were pelleted (6000 g, 20 min, 4° C.), resuspended in 1× PBS, and lysed with a high-pressure homogenizer. The insoluble fraction was removed with centrifugation (15000 g, 20 min, 4° C.).

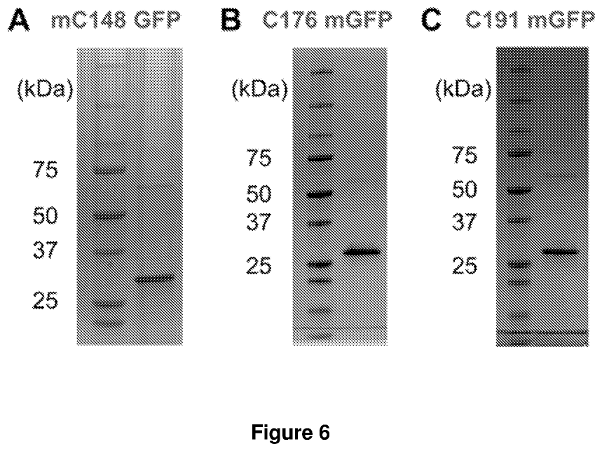

The mGFP mutants have a polyhistidine tag, which was used to isolate the mutants from cell lysate using nickel affinity chromatography. Proteins were loaded onto a column packed with Profinity™ IMAC Resin (Bio-Rad). The column was washed with 100 mL of 1× PBS with 12.5 mM imidazole and proteins were eluted with 15 mL of 1× PBS with 250 mM imidazole. The mGFP mutants were separated from the imidazole using anion exchange chromatography. The proteins were then loaded onto a column packed with Macro-Prep® DEAE resin (Bio-Rad). The column was washed with 40 mL of 1× PBS and proteins were eluted with 15 mL of 1× PBS with an additional 250 mM NaCI. Protein purity was confirmed with SDS PAGE, showing mGFP primarily as monomers with small impurities of dimers that are formed from the oxidation of cysteine to form a disulfide bond (FIG. 6).

Oligonucleotide design and synthesis. Nine DNA sequences or pairs of complementary DNA sequences were designed to study how DNA interactions can influence protein crystallization and packing into single crystals (Table 2). DNA designs varied between self-complementary (scDNA), complementary (cDNA), and non-complementary (ncDNA). DNA length varied between 6 and 18 bases. The sites with the DNA for attachment to mGFP was either at an internal or external position on the DNA strand.

Oligonucleotides utilized herein were synthesized on solid supports using reagents obtained from Glen Research and standard protocols (Table 2). Products were cleaved from the solid support using 15% (w/v) ammonium hydroxide (aq) and 20% (w/v) methyl amine for 20 min at 55° C. and purified using reverse-phase HPLC with a gradient of 0 to 75 percent acetonitrile in triethylammonium acetate buffer over 45 min. Dimethoxytrityl or monomethoxytrityl groups were cleaved with 20% (v/v) acetic acid for 2 h and extracted with ethyl acetate. The masses of the oligonucleotides were confirmed using matrix-assisted laser desorption ionization mass spectrometry (MALDI-MS) using 3-hydroxypicolinic acid, 2′5′-dihydroxyacetophenone, or 2′,4′,6′-trihydroxyacetophenone monohydrate as a matrix. All synthesized DNA masses were within 30 DA of the expected mass.

TABLE 2
Oligonucleotide sequence designs. Extinction coefficients
and expected molecular weights (MWexpected) were
calculated with the IDT OligoAnalyzer Tool (Integrated
DNA Technologies). Experimental molecular weights
(MWexperimental) were measured with MALDI-MS.

| Name | Sequence (5′ → 3′) | ε(M⁻¹ cm⁻¹) | MW_{expected} (Da) | MWₑₓₚₑᵣᵢₘₑₙₜₐₗ (Da) |
|---|---|---|---|---|
| scDNA-1 | H₂N-CGCGCG | 51400 | 1930.2 | 1960.3 |
| cDNA-1 | H₂N-GGCCGG | 55600 | 2012.4 | 2002.0 |
| | H₂N-CCGGCC | 48600 | 1932.3 | 1919.3 |
| cDNA-2 | H₂N-AGAGAG | 71600 | 2044.4 | 2046.0 |
| | H₂N-CTCTCT | 45800 | 1897.3 | 1898.3 |
| ncDNA-1 | H₂N-TTTTTT | 49200 | 1942.4 | 1929.9 |
| cDNA-3 | H₂N-AAGGAAGGA | 106200 | 3000.1 | 3005.9 |
| | H₂N-TCCTTCCTT | 69900 | 2794.9 | 2797.4 |
| cDNA-4 | H₂N-AAGGAAGGAAGG | 137900 | 3971.7 | 3981.5 |
| | (SEQ ID NO: 4) | | | |
| | H₂N-CCTTCCTTCCTT | 91700 | 3677.5 | 3677.5 |
| | (SEQ ID NO: 5) | | | |
| cDNA-5 | H₂N-AGTTAGGACTTACGCTAC | 176900 | 5677.8 | 5684.8 |
| | (SEQ ID NO: 6) | | | |
| | H₂N-GTAGCGTAAGTCCTAACT | 177100 | 5677.8 | 5683.3 |
| | (SEQ ID NO: 7) | | | |
| ncDNA-2 | H₂N-TTTTTTTTT | 73500 | 2855.0 | 2889.1 |
| scDNA-2 | GCGCT(NH₂)AGC | 80600 | 2508.8 | 2510.2 |

H₂N- = 5′ Amino C6 modifier
T(NH₂) = Amino C2 dT modifier

Synthesis, Purification, and Characterization of mGFP-DNA Conjugates.

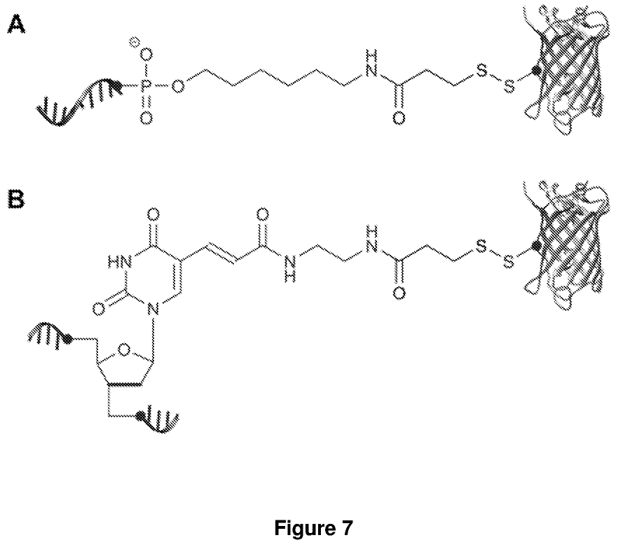

Conjugation of mGFP and DNA was performed according to a previously published procedure [Hayes, O. G., McMillan, J. R., Lee, B., and Mirkin, C. A. (2018). DNA-Encoded Protein Janus Nanoparticles. J. Am. Chem. Soc. 140, 9269-9274]. Linkage structures for mGFP-DNA are depicted in FIG. 7. Amine-modified DNA (3000 nmol) was reacted with 30-50 equivalents of succinimidyl 3-(2-pyridyldithio)propionate (SPDP, ThermoFisher) in 50:50 DMF:1× PBS, pH 7.4 for 1 hour at RT. DNA was purified from excess SPDP with two consecutive illustra NAP Columns (GE Healthcare Life Sciences). The purified DNA was reacted with mGFP (300 nmol) overnight at RT with 300 rpm shaking. The reaction mixture was loaded onto a column packed with Profinity™ IMAC Resin (Bio-Rad). To remove unreacted DNA, the column was washed with 40 mL of 1× PBS. Protein and protein-DNA conjugates were eluted with 15 mL of 1× PBS with 250 mM imidazole. The eluent was then loaded onto a column packed with Macro-Prep® DEAE resin (Bio-Rad). The column was washed with 40 mL of 1× PBS and 30 mL of 1× PBS with an additional 200 mM NaCI to remove thiol and disulfide forms of mGFP. Conjugates of mGFP-DNA were eluted with 15 mL of 1× PBS with an additional 500 mM NaCI.

Synthesis and purity of mGFP-DNA conjugates were confirmed with UV-vis absorption spectroscopy, SDS PAGE, and MALDI-MS (see below for mGFP-DNA conjugate characterization data). The C148 mGFP, C176 mGFP, and C191 mGFP mutants show absorption maxima at 488 nm (ε=55000 M⁻¹ cm⁻¹) due to the mGFP chromophore [Patterson, G. H., Knobel, S. M., Sharif, W. D., Kain, S. R., and Piston, D. W. (1997). Use of the green fluorescent protein and its mutants in quantitative fluorescence microscopy. Biophys. J. 73, 2782-2790] and at 280 nm due to aromatic amino acid side chains [Gill, S. C., and von Hippel, P. H. (1989). Calculation of protein extinction coefficients from amino acid sequence data. Anal. Biochem. 182, 319-326]. DNA shows an absorption maxima around 260 nm and extinction coefficients at 260 nm were calculated with the IDT OligoAnalyzer Tool (Integrated DNA Technologies). After purification of mGFP-DNA conjugates, the number of DNA per mGFP in solution was quantified by comparing the relative absorption at 488 nm and 260 nm for mGFP and mGFP-DNA. The increase in mass of mGFP-DNA conjugates after DNA functionalization and sample purity was confirmed with SDS PAGE using 4-15% Mini-PROTEAN® TGX™ Precast Protein Gels (Bio-Rad) and a Precision Plus Protein™ All Blue Prestained Protein Standard (Bio-Rad). The increase in mass of mGFP-DNA conjugates after DNA conjugation and sample purity was also confirmed with MALDI-MS. Before MALDI-MS, conjugates of mGFP-DNA were transferred to water 6 times using 30 kDa cutoff Amicon® Ultra-0.5 mL Centrifugal Filters (Millepore Sigma) and mixed with MALDI matrix 2′5¹ dihydroxyacetophenone, 2′,4′,6′-trihydroxyacetophenone monohydrate, or sinapinic acid.

Crystallization of mGFP-DNA and x-ray crystallography. Using 30 kDa cutoff Amicon® Ultra-15 Centrifugal Filter Units (Millepore Sigma), all mGFP-DNA conjugates were buffer exchanged 4 times to 10 mM Tris with 137 mM NaCI and concentrated to 5 mg/mL (protein concentration). High-throughput sitting drop vapor diffusion experiments were set up with Crystal Gryphon (Art Robbins Instruments) or mosquito® crystal (TTP Labtech) liquid handlers in 96-3 well INTELLI-PLATE® trays (Art Robbins Instruments). The reservoirs consisted of 704 of crystallization condition and the sitting drops consisted of 14 of sample and 1 μL of crystallization condition. Crystallization conditions from the PACT, JCSG+, Classics II, and PEGs II Suites (Qiagen) were screened. These condition suites vary salt identity and concentration, buffer identity and concentration, pH, and precipitant identity and concentration. Crystallization experiments at both 4 and 22° C. proceeded for 2 weeks undisturbed. Obtained crystals were transferred to nylon loops and frozen in liquid nitrogen. X-ray diffraction experiments were performed at the Life Sciences Collaborative Access Team beamlines 21-ID-D, 21-ID-F, and 21-ID-G at the Advanced Photon Source, Argonne National Laboratory.

Solving mGFP-DNA crystal structures. Diffraction data were processed with programs run through Xia2 [Evans, P. R., and Murshudov, G. N. (2013). How good are my data and what is the resolution? Acta Crystallogr., Sect. D: Biol. Crystallogr. 69, 1204-1214; Winter, G. (2010). xia2: an expert system for macromolecular crystallography data reduction. J. Appl. Crystallogr. 43, 186-190] or programs from the CCP4 software suite [Winn, M. D., Ballard, C. C., Cowtan, K. D., Dodson, E. J., Emsley, P., Evans, P. R., Keegan, R. M., Krissinel, E. B., Leslie, A.G.W., McCoy, A., et al. (2011). Overview of the CCP4 suite and current developments. Acta Crystallogr., Sect. D: Biol. Crystallogr. 67, 235-242]. Data were indexed and integrated with iMosflm [Battye, T. G. G., Kontogiannis, L., Johnson, O., Powell, H. R., and Leslie, A. G. W. (2011). iMOSFLM: a new graphical interface for diffraction-image processing with MOSFLM. Acta Crystallogr., Sect. D: Biol. Crystallogr. 67, 271-281], and space group and unit cell parameters were confirmed with Pointless [Evans, P. (2006). Scaling and assessment of data quality. Acta Crystallogr., Sect. D: Biol. Crystallogr. 62, 72-82]. After scaling and merging data with SCALA [Evans, P. (2011). An introduction to data reduction: space-group determination, scaling and intensity statistics. Acta Crystallogr., Sect. D: Biol. Crystallogr. 67, 282-292], structures were determined by molecular replacement with PhaserMR [McCoy, A. J., Grosse-Kunstleve, R. W., Adams, P. D., Winn, M. D., Storoni, L. C., and Read, R. J. (2007). Phaser crystallographic software. J. Appl. Crystallogr. 40, 658-674], using GFP (5N90 or 4EUL) as the starting model [Kachalova, G.S., Popov, A.P., Simanovskaya, A.A., and Lipkin, A.V. (2018). Structure of EGFP(enhanced green fluorescent protein) mutant—L232H at 0.153 nm. To Be Published; Arpino, J. A. J., Rizkallah, P. J., and Jones, D. D. (2012). Crystal Structure of Enhanced Green Fluorescent Protein to 1.35 Å Resolution Reveals Alternative Conformations for Glu222. PLoS One 7, e47132]. After successive rounds of manual model building and addition of water molecules with Coot [Emsley, P., Lohkamp, B., Scott, W. G., and Cowtan, K. (2010). Features and development of Coot. Acta Crystallogr., Sect. D: Biol. Crystallogr. 66, 486-501] and refinement with Refmac5 [Murshudov, G. N., Skubak, P., Lebedev, A. A., Pannu, N. S., Steiner, R. A., Nicholls, R. A., Winn, M. D., Long, F., and Vagin, A. A. (2011). REFMACS for the refinement of macromolecular crystal structures. Acta Crystallogr., Sect. D: Biol. Crystallogr. 67, 355-367], structures were deemed finalized when Rwork/Rfree values plateaued. Protein and water B-factor analyses were performed using the bavarage module in the CCP4 software suite. Graphics for protein crystal structures were generated using PyMOL [Schrodinger, LLC (2015). The PyMOL Molecular Graphics System, Version 1.8], UCSF Chimera [Pettersen, E. F., Goddard, T. D., Huang, C. C., Couch, G. S., Greenblatt, D. M., Meng, E. C., and Ferrin, T. E. (2004). UCSF Chimera—A visualization system for exploratory research and analysis. J. Comput. Chem. 25, 1605-1612], and QuteMol [Tarini, M., Cignoni, P., and Montani, C. (2006). Ambient Occlusion and Edge Cueing for Enhancing Real Time Molecular Visualization. IEEE Trans Vis Comput Graph 12, 1237-1244].

Confocal microscopy. Crystals were transferred from sitting drops to a 7 μL drop of crystallization condition on a confocal microscopy dish. Crystals were imaged with a Nikon A1R confocal microscope using a 20× objective with bright field and two laser channels. The first channel for the mGFP chromophore (488 nm excitation maximum, 509 emission maximum) was excited with a 485 nm laser and had an emission filter of 500 — 550 nm. The second channel for the DNA intercalating dye, TOTO-3, (642 nm excitation maximum, 662 emission maximum) [Rye, H. S., Yue, S., Wemmer, D. E., Quesada, M. A., Haugland, R. P., Mathies, R. A., and Glazer, A. N. (1992). Stable fluorescent complexes of double-stranded DNA with bis-intercalating asymmetric cyanine dyes: properties and applications. Nucleic Acids Res. 20, 2803-2812; Nygren, J., Svanvik, N., and Kubista, M. (1998). The interactions between the fluorescent dye thiazole orange and DNA. Biopolymers 46, 39-51] was excited with a 640 nm laser and had an emission filter of 663-738 nm. After imaging the crystals, TOTO-3 (1 mM in DMSO, Biotium) was diluted to 0.1 mM in 10 mM Tris with 137 mM NaCI and 0.5 μL of the diluted dye was added to the drop containing the crystals. After waiting 30 minutes for the dye to diffuse through the crystals, the crystals were imaged again with the same bright field and laser channels.

TABLE 3
Crystal structure table with data collection and processing information.

| | Sample | | | |
|---|---|---|---|---|
| | | | C148 mGFP − | C148 mGFP + |
| | C148 mGFP | C176 mGFP | scDNA-1 | scDNA-1 |
| | (Thiol Form) | (Disulfide Form) | (6 bp) | (6 bp) |
| Table 1 Line Number | 1 | 2 | 4 | 5 |
| PDB Code | 6UHJ | 6UHK | 6UHL | 6UHM |
| Cell parameters (Å) | 51.5, 62.9, 69.4 | 88.9, 91.8, 151.7 | 64.9, 52.3, 86.8 | 58.3, 61.8, 135.3 |
| Cell parateters (°) | 90, 90, 90 | 90, 90, 90 | 90, 94, 90 | 90, 90, 90 |
| Space group | P2₁2₁2₁ | I222 | P2₁ | P2₁2₁2₁ |
| Crystallization | PEGs II Suite | JCSG+ Suite | PACT Suite | PEGs II Suite |
| Condition | Condition | Condition | Condition | Condition |
| | F8 at 22° C. | F8 at 22° C. | A5 at 22° C. | F8 at 22° C. |
| Resolution | 51.51-1.50 | 78.51-1.90 | 43.29-1.91 | 61.76-2.10 |
| range^{a} (Å) | (1.58-1.50) | (2.00-1.90) | (1.96-1.91) | (2.21-2.10) |
| Wavelength (Å) | 0.97857 | 0.97872 | 0.97872 | 0.97872 |
| Observed hkl | 163324 | 727590 | 435564 | 250177 |
| Unique hkl | 36338 | 96006 | 43945 | 29215 |
| Redundancy | 4.5 (3.9) | 7.6 (7.5) | 9.9 (5.8) | 8.6 (8.7) |
| Completeness (%) | 98.8 (98.0) | 100.0 (99.9) | 96.9 (67.2) | 99.8 (99.4) |
| Mean (I/σ(I)) | 10.7 (3.6) | 14.7 (3.8) | 12.1 (1.3) | 14.5 (3.7) |
| R_{sym}^{b} (%) | 0.083 (0.347) | 0.082 (0.515) | 0.117 (1.189) | 0.074 (0.503) |

^{a}Numbers in parentheses refer to the highest-resolution shell,
^{b}R_{sym} = Σh Σi|I₁(h) − <I(h)|/Σh Σi I₁(h).

TABLE 4
Crystal structure table with data collection and processing information.

| | Sample | | | |
|---|---|---|---|---|
| | C148 mGFP − | C148 mGFP − | C148 mGFP − | C148 mGFP − |
| | cDNA-1 | cDNA-2 | ncDNA-1 | cDNA-3 |
| | (6 bp) | (6 bp) | (6 bp) | (9 bp) |
| Table 1 Line Number | 6 | 7 | 8 | 9 |
| PDB Code | 6UHN | 6UHO | 6UHP | 6UHQ |
| Cell parameters (Å) | 64.7, 52.2, 86.5 | 64.7, 52.2, 86.4 | 59.1, 51.6, 100.4 | 106.6, 50.6, 56.7 |
| Cell parateters (°) | 90, 94, 90 | 90, 90, 90 | 90, 107, 90 | 90, 110, 90 |
| Space group | P2₁ | P2₁ | P2₁ | C2 |
| Crystallization | JCSG+ Suite | JCSG+ Suite | Classics II Suite | PEGs II Suite |
| Condition | Condition | Condition | Condition | Condition |
| | H9 at 22° C. | G10 at 22° C. | F10 at 4° C. | D7 at 22° C. |
| Resolution | 64.07-1.92 | 64.53-1.95 | 56.48-2.90 | 53.16-2.85 |
| range^{a} (Å) | (2.02-1.92) | (2.06-1.95) | (3.06-2.90) | (3.00-2.85) |
| Wavelength (Å) | 0.97872 | 0.97872 | 0.97857 | 0.97872 |
| Observed hkl | 191327 | 179992 | 66203 | 21886 |
| Unique hkl | 43273 | 41971 | 13070 | 6723 |
| Redundancy | 4.4 (4.4) | 4.3 (4.3) | 5.1 (5.2) | 3.3 (3.2) |
| Completeness (%) | 99.1 (98.3) | 99.2 (100.0) | 99.8 (100.0) | 99.3 (99.2) |
| Mean (I/σ(I)) | 4.9 (1.0) | 9.4 (3.3) | 7.9 (2.8) | 5.8 (2.1) |
| R_{sym}^{b} (%) | 0.207 (1.535) | 0.094 (0.406) | 0.147 (0.657) | 0.178 (0.677) |

^{a}Numbers in parentheses refer to the highest-resolution shell,
^{b}R_{sym} = Σh Σi|I₁(h) − <I(h)|/Σh Σi I₁(h).

TABLE 5
Crystal structure table with data collection
and processing information.

| | Sample |
|---|---|
| | C148 mGFP − scDNA-2 |
| | (8 bp, int. DNA attach.) |
| Table 1 Line Number | 15 |
| PDB Code | 6UHR |
| Cell parameters (Å) | 50.6, 50.9, 209.2 |
| Cell parameters (°) | 90, 90, 90 |
| Space group | P2₁2₁2₁ |
| Crystallization Condition | Classics II Suite Condition |
| | H12 at 22° C. |
| Resolution range^{a} (Å) | 69.73-3.00 |
| | (3.16-3.00) |
| Wavelength (Å) | 1.12710 |
| Observed hkl | 79211 |
| Unique hkl | 11425 |
| Redundancy | 6.9 (7.1) |
| Completeness (%) | 99.7 (100.0) |
| Mean (I/σ(I)) | 8.1 (2.7) |
| R_{sym}^{b} (%) | 0.194 (0.716) |

^{a}Numbers in parentheses refer to the highest-resolution shell,
^{b}R_{sym} = Σh Σi|I₁(h) − <I(h)|/Σh Σi I₁(h).

TABLE 6
Crystal structure table with data refinement information.

| | Sample | | | |
|---|---|---|---|---|
| | | | C148 mGFP − | C148 mGFP + |
| | C148 mGFP | C176 mGFP | scDNA-1 | scDNA-1 |
| | (Thiol Form) | (Disulfide Form) | (6 bp) | (6 bp) |
| Table 1 Line Number | 1 | 2 | 4 | 5 |
| PDB Code | 6UHJ | 6UHK | 6UHL | 6UHM |
| Resolution range^{a} (Å) | 46.61-1.50 | 75.97-1.90 | 43.29-1.91 | 56.25-2.10 |
| | (1.54-1.50) | (1.95-1.90) | (1.96-1.91) | (2.16-2.10) |
| No. of Reflections | 36338 | 96006 | 43939 | 29157 |
| R factor^{c} | 15.3 | 18.6 | 22.8 | 21.1 |
| R_{free}^{d} | 18.5 | 22.9 | 27.3 | 27.0 |
| RMSD bond lengths (Å) | 0.013 | 0.012 | 0.0094 | 0.008 |
| RMSD bond angles (°) | 1.88 | 1.86 | 1.78 | 1.67 |
| Average B value Protein (Å²) | 10.5 | 32.4 | 21.1 | 52.2 |
| Average B value Water (Å²) | 25.8 | 39.9 | 30.1 | 53.9 |
| Ramachandran Plot (%) | | | | |
| Favored and allowed regions | 96.7 | 95.4 | 96.8 | 93.9 |
| Generously allowed regions | 3.3 | 4.4 | 3.2 | 5.4 |
| Disallowed regions | 0.0 | 0.2 | 0.0 | 0.7 |

^{c}R factor = Σₕₖₗ||F_{obs}| − k|F_{calc}||/Σₕₖₗ|F_{obs}|,
^{d}R_{free} is calculated using the same equation as that for R factor, but 5.0% of reflections were chosen randomly and omitted from the refinement.

TABLE 7
Crystal structure table with data refinement information.

| | Sample | | | |
|---|---|---|---|---|
| | C148 mGFP − | C148 mGFP − | C148 mGFP − | C148 mGFP − |
| | cDNA-1 | cDNA-2 | ncDNA-1 | cDNA-3 |
| | (6 bp) | (6 bp) | (6 bp) | (9 bp) |
| Table 1 Line Number | 6 | 7 | 8 | 9 |
| PDB Code | 6UHN | 6UHO | 6UHP | 6UHQ |
| Resolution range (Å) | 64.60-1.92 | 64.53-1.95 | 56.48-2.90 | 50.04-2.85 |
| | (1.97-1.92) | (2.00-1.95) | (2.98-2.90) | (2.92-2.85) |
| No. of Reflections | 44378 | 41801 | 12900 | 6722 |
| R factor^{c} | 21.6 | 21.4 | 34.1 | 18.1 |
| R_{free}^{d} | 24.7 | 25.1 | 37.3 | 27.4 |
| RMSD bond lengths (Å) | 0.011 | 0.010 | 0.007 | 0.007 |
| RMSD bond angles (°) | 1.83 | 1.78 | 1.63 | 1.75 |
| Average B value Protein (Å²) | 24.5 | 27.1 | 55.4 | 33.3 |
| Average B value Water (Å²) | 28.6 | 36.0 | n/a | 24.0 |
| Ramachandran Plot (%) | | | | |
| Favored and allowed regions | 96.5 | 95.9 | 82.3 | 92.8 |
| Generously allowed regions | 3.5 | 3.6 | 12.3 | 7.2 |
| Disallowed regions | 0.0 | 0.5 | 5.5 | 0.0 |

^{c}R factor = Σₕₖₗ||F_{obs}| − k|F_{calc}||/Σₕₖₗ|F_{obs}|,
^{d}R_{free} is calculated using the same equation as that for R factor, but 5.0% of reflections were chosen randomly and omitted from the refinement.

TABLE 8
Crystal structure table with data refinement information.

| | Sample |
|---|---|
| | C148 mGFP − scDNA-2 |
| | (8 bp, int. DNA attach.) |
| Table 1 Line Number | 15 |
| PDB Code | 6UHR |
| Resolution range (Å) | 52.30-3.00 |
| | (3.08-3.00) |
| No. of Reflections | 11431 |
| R factor^{c} | 22.6 |
| R_{free}^{d} | 30.9 |
| RMSD bond lengths (Å) | 0.008 |
| RMSD bond angles (°) | 1.82 |
| Average B value Protein (Å²) | 47.4 |
| Average B value Water (Å²) | 40.7 |
| Ramachandran Plot (%) | |
| Favored and allowed regions | 84.4 |
| Generously allowed regions | 11.8 |
| Disallowed regions | 3.9 |

^{c}R factor = Σₕₖₗ||F_{obs}| − k|F_{calc}||/Σₕₖₗ|F_{obs}|,
^{d}R_{free} is calculated using the same equation as that for R factor, but 5.0% of reflections were chosen randomly and omitted from the refinement.

### Example 4

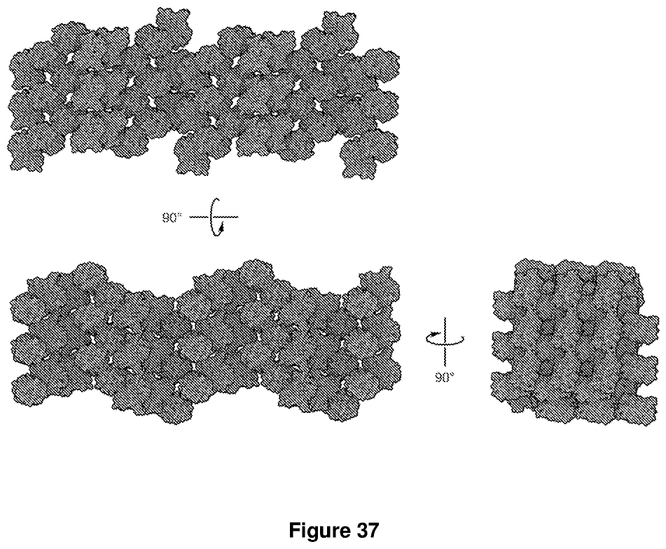

This example utilizes as a model the mutant green fluorescent protein (mGFP). The effects of design parameters, including DNA sequence, DNA length, protein amino acid attachment position, and DNA base attachment position were systematically explored with respect to consequence on protein packing in the crystals (FIG. 37). Importantly, for many of the systems studied, x-ray diffraction quality single crystals could be obtained, and an elucidation of the resulting structures provided insight into the design parameters that control protein packing within such crystals. Taken together, the data demonstrated that a single DNA modification on the surface of a protein can be used to direct protein packing within a single crystal and, as such, is an important step forward in protein crystal engineering.

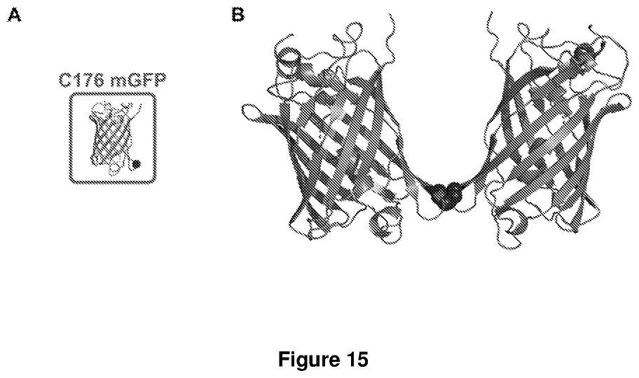

To study how designed DNA interactions can influence the growth and packing of protein single crystals, GFP mutants were designed that could be modified with one DNA strand using cysteine-conjugation methods. A single cysteine residue was positioned at a distinct surface location on both mutants, either on the side (C148 mGFP) [Hayes, O. G., McMillan, J. R., Lee, B., and Mirkin, C. A. (2018). DNA-Encoded Protein Janus Nanoparticles. J. Am. Chem. Soc. 140, 9269-9274] or the edge (C176 mGFP or C191 mGFP) of the mGFPβ-barrel (Table 1). Crystal structures of C148 mGFP and C176 mGFP were determined (a structure of C191 mGFP is known) [Leibly, D. J., Arbing, M. A., Pashkov, I., DeVore, N., Waldo, G. S., Terwilliger, T. C., and Yeates, T. O. (2015). A Suite of Engineered GFP Molecules for Oligomeric Scaffolding. Structure 23, 1754-1768] prior to their functionalization with DNA as comparisons to structures obtained when DNA is present. While crystal structures of native GFP are well known [Arpino, J. A. J., Rizkallah, P. J., and Jones, D. D. (2012). Crystal Structure of Enhanced Green Fluorescent Protein to 1.35 Å Resolution Reveals Alternative Conformations for Glu222. PLoS One 7, e47132], the position of solvent-accessible cysteine residues on mGFP influences protein packing through the formation of disulfide bonds [Leibly, D. J., Arbing, M. A., Pashkov, I., DeVore, N., Waldo, G. S., Terwilliger, T. C., and Yeates, T. O. (2015). A Suite of Engineered GFP Molecules for Oligomeric Scaffolding. Structure 23, 1754-1768]. The C148 mGFP was crystallized, and a 1.5 Å structure where C148 remains as a thiol was determined in the space group P2₁2₁2₁ (FIG. 2a, 6UHJ). The structure is nearly identical to the majority of GFP structures in the Protein Data Bank (PDB) [Berman, H. M., Westbrook, J., Feng, Z., Gilliland, G., Bhat, T. N., Weissig, H., Shindyalov, I. N., and Bourne, P. E. (2000). The Protein Data Bank. Nucleic Acids Res. 28, 235-242], with nearly equivalent unit cell parameters and a root-mean-square deviation (rmsd) of 0.2 Å for all atoms from the GFP structure 4EUL [Arpino, J. A. J., Rizkallah, P. J., and Jones, D. D. (2012). Crystal Structure of Enhanced Green Fluorescent Protein to 1.35 Å Resolution Reveals Alternative Conformations for Glu222. PLoS One 7, e47132]. Crystals of C176 mGFP were characterized where C176 form disulfide bonds (product of oxidation) as a novel structure in the space group 1222 at 1.9 Å resolution (FIG. 15, 6UHK).

Next, the effect of introducing a designed DNA interaction between proteins on crystallization and protein packing was investigated within a single crystal. Design parameters including DNA sequence, DNA length, amino acid attachment position, and DNA base attachment position, were varied. In a typical experiment, the surface cysteine on mGFP was functionalized with pyridyl disulfide-modified DNA (mGFP-DNA) through a thiol-disulfide exchange reaction according to previously published procedures (See Materials and Methods, above) [Hayes, O. G., McMillan, J. R., Lee, B., and Mirkin, C. A. (2018). DNA-Encoded Protein Janus Nanoparticles. J. Am. Chem. Soc. 140, 9269-9274]. Unreacted DNA and protein were removed using nickel affinity and anion exchange chromatography, respectively. Mono-functionalization of mGFP with DNA and purification of mGFP-DNA conjugates were confirmed using UV-vis spectroscopy, SDS-PAGE, and matrix-assisted laser desorption ionization mass spectrometry (MALDI-MS). These data conclusively demonstrated the attachment of DNA to mGFP and the purification of the mGFP-DNA conjugates. The mGFP-DNA conjugates were crystallized using vapor diffusion techniques and hundreds of crystallization conditions (varying salt, precipitant, buffer, and temperature) were screened robotically in a high-throughput manner. The protein packing within each single crystal was characterized with x-ray crystallographic structure determination.

A mGFP-DNA Single Crystal Structure

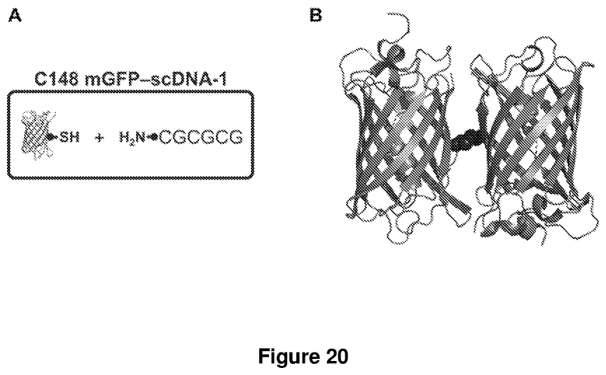

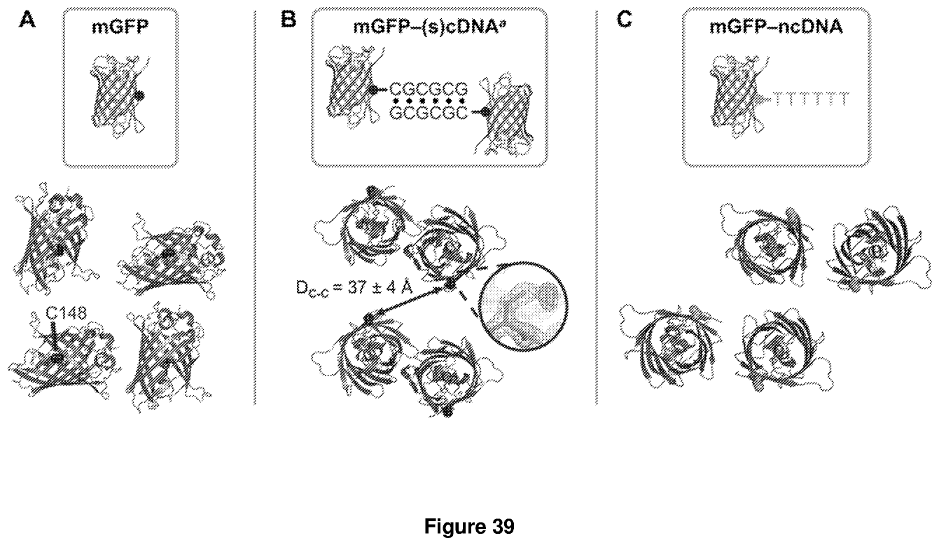

As a proof-of-concept that DNA interactions can modify the growth and packing of protein single crystals, the crystallization of mGFP modified with a 6 base pair (bp) self-complementary DNA strand (scDNA-1) at the C148 position (mGFP-scDNA-1, Table 9: Line 4) was first studied. DNA conjugation did not inhibit the protein's ability to crystallize, as the mGFP-scDNA-1 conjugate crystallized into thin plates (˜100 μm×200 μm×10 μm). Significantly, a 1.9 Å resolution crystal structure in the space group P2₁ was determined (FIG. 39b, 6UHL). Furthermore, the structure has different unit cell parameters and protein packing with respect to the C148 mGFP crystal structure, indicating that the DNA modification plays a role in how the proteins are organized. In fact, the unit cell parameters and protein packing in the mGFP-scDNA-1 crystal are novel relative to all previously reported GFP crystal structures. The crystal structure shows electron density for mGFP and the disulfide mGFP-scDNA-1 attachment, but not DNA. The flexibility of the linker used for protein conjugation (see FIG. 7 for linkage structure) likely prevents DNA from ordering in the crystal. However, the mGFP—scDNA-1 protein packing is consistent with the presence of hybridized DNA. Pairs of C148 residues orient towards distinct regions of solvent space and are separated by 37±4 A, a distance that corresponds well with the length of the duplexed DNA within the protein single crystals (theoretical distance for 6 bp duplex DNA is 27-64 A, either in contracted/extended form with respect to the two alkyl linker molecules) [Wing, R., Drew, H., Takano, T., Broka, C., Tanaka, S., Itakura, K., and Dickerson, R. E. (1980). Crystal structure analysis of a complete turn of B-DNA. Nature 287, 755-758]. As an additional control experiment to confirm that covalent attachment of scDNA-1 to mGFP directs the new mGFP—scDNA-1 crystal structure, a physical mixture of C148 mGFP and scDNA-1 was subjected to identical crystallization conditions as the conjugate (Table 9: Line 5). The crystals resulting from the physical mixture show a structure with a disulfide bond between surface cysteines (6UHM, FIG. 20), where mGFP packing is exclusively directed by inter-protein interactions. Taken together, these results showed that the covalent attachment of a 6 bp self-complementary DNA strand to mGFP leads to a change in protein—protein contacts during crystallization and, ultimately, novel protein packing.

TABLE 9
Sample designs for mGFP-DNA conjugates that are used to
study the effect of DNA sequence, DNA length, amino acid
attachment position, and DNA base attachment position on
protein packing within single crystals.

| Line | | PDB | mGFP | | |
|---|---|---|---|---|---|
| Number | Sample | Code | mutant | DNA Design | Study |
| 1 | mGFP | 6UHJ | C148 | n/a | Control |
| 2 | mGFP | 6UHK | C176 | n/a | Control |
| 3 | mGFP | n/a | C191 | n/a | Control |
| 4 | mGFP-scDNA-1^{a} | 6UHL | C148 | H₂N-CGCGCG | DNA sequence |
| | | | | | DNA length |
| | | | | | Amino acid |
| | | | | | attachment |
| | | | | | position |
| | | | | | DNA base |
| | | | | | attachment |
| | | | | | position |
| 5 | mGFP + scDNA-1 | 6UHM | C148 | H₂N-CGCGCG | Control |
| 6 | mGFP-cDNA-1^{b} | 6UHN | C148 | H₂N-GGCCGG, | DNA sequence |
| | | | | H₂N-CCGGCC | |
| 7 | mGFP-cDNA-2 | 6UHO | C148 | H₂N-AGAGAG, | DNA sequence |
| | | | | H₂N-CTCTCT | |
| 8 | mGFP-ncDNA-1^{c} | 6UHP | C148 | H₂N-TTTTTT | DNA sequence |
| 9 | mGFP-cDNA-3 | 6UHQ | C148 | H₂N-AAGGAAGGA, | DNA length |
| | | | | H₂N-TCCTTCCTT | |
| 10 | mGFP-cDNA-4 | Did not | | H₂N-AAGGAAGGAAGG | DNA length |
| | | crystallize | C148 | (SEQ ID NO: 4), | |
| | | | | H₂N-CCTTCCTTCCTT | |
| | | | | (SEQ ID NO: 5) | |
| 11 | mGFP-cDNA-5 | Did not | C148 | H₂N-AGTTAGGACTTA | DNA length |
| | | crystallize | | CGCTAC | |
| | | | | (SEQ ID NO: 6), | |
| | | | | H₂N-GTAGCGTAAGTC | |
| | | | | CTAACT | |
| | | | | (SEQ ID NO: 7) | |
| 12 | mGFP-ncDNA-2 | Did not | C148 | H₂N-TTTTTTTTT | DNA length |
| | | crystallize | | | |
| 13 | mGFP-scDNA-1 | Did not | C176 | H₂N-CGCGCG | Amino acid |
| | | crystallize | | | attachment |
| | | | | | position |
| 14 | mGFP-scDNA-1 | Did not | C191 | H₂N-CGCGCG | Amino acid |
| | | crystallize | | | attachment |
| | | | | | position |
| 15 | mGFP-scDNA-2 | 6UHR | C148 | GCGCT(NH₂)AGC | DNA base |
| | | | | | attachment |
| | | | | | position |

DNA Hybridization Directs mGFP-DNA Packing

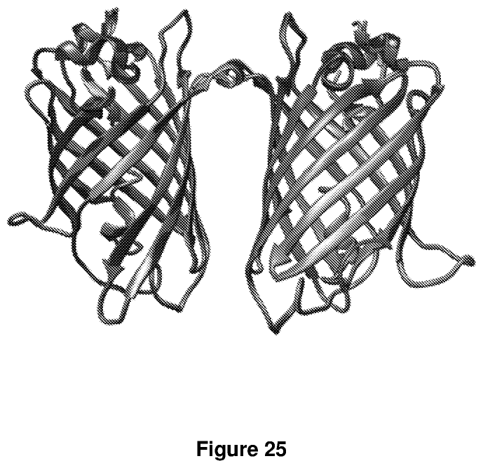

To explore whether DNA-directed protein packing using complementary strands is independent of specific sequence, two sets of 6 bp complementary DNA were designed (cDNA-1 and cDNA-2, Table 9: Lines 6 and 7). The C148 mGFP was functionalized with the complementary DNA sequences separately, then corresponding mGFP-DNA conjugates were mixed immediately prior to subjecting the mixture to crystallization experiments. Both mGFP—cDNA-1 and mGFP-cDNA-2 crystallized into thin plates, showing the same crystal morphology as mGFP—scDNA-1 crystals. Furthermore, 1.9 Å crystal structures for mGFP-cDNA-1 and mGFP-cDNA-2 have the same space group P2₁ and nearly equivalent unit cell parameters as the mGFP—scDNA-1 structure (FIG. 39b, 6UHN and 6UHO, respectively). The rmsd between mGFP—scDNA-1, mGFP-cDNA-1, and mGFP-cDNA-2 structures are less than 0.2 Å for all atoms, confirming that the protein packing of these structures is essentially equivalent (FIG. 25). Therefore, (self-)complementary mGFP-DNA conjugates with a DNA length of 6 bp crystallize into practically identical single crystal forms, regardless of DNA sequence.

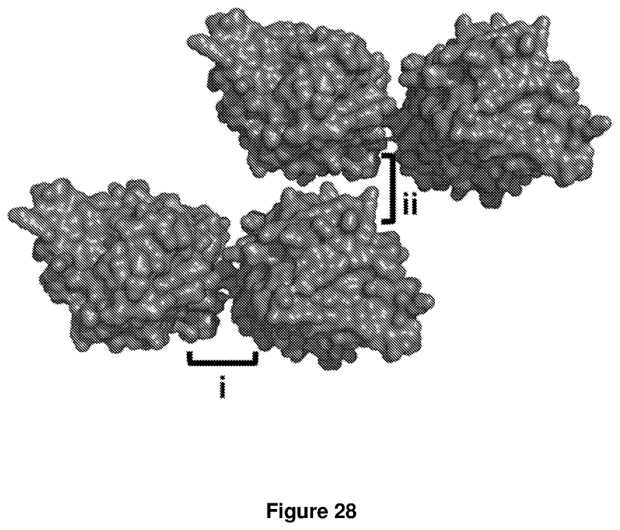

Next, the importance of DNA complementarity on the resulting crystal structure was confirmed. The C148 mGFP was functionalized with a T6 non-complementary DNA strand (mGFP—ncDNA-1, Table 9: Line 8) and crystallized. The mGFP—ncDNA-1 conjugates formed needle-like crystals, a distinct crystal morphology from mGFP and the three 6 bp (self-) complementary mGFP-DNA conjugates. Moreover, a 2.9 Å resolution crystal structure in the space group P2₁ was determined for mGFP—ncDNA-1 with unit cell parameters and protein packing that are different from both those of mGFP and (self-)complementary mGFP-DNA conjugates (FIG. 39c, 6UHP). Clearly, the presence of non-complementary single stranded DNA still influences packing outcomes of mGFP, likely by filling space and altering the crystal contacts that may form between mGFP. However, the protein packing in the mGFP—ncDNA-1 structure is not consistent with DNA duplexing, as each C148 residue orients towards a different region of solvent space with no free path in solvent space between C148 residues that would permit DNA hybridization (FIG. 28). This result indicated the importance of DNA complementarity on protein packing outcomes in protein—DNA crystals and illustrates that protein packing within single crystals (mGFP—scDNA-1, mGFP-cDNA-1, and mGFP-cDNA-2) can be directed using programmable DNA interactions.

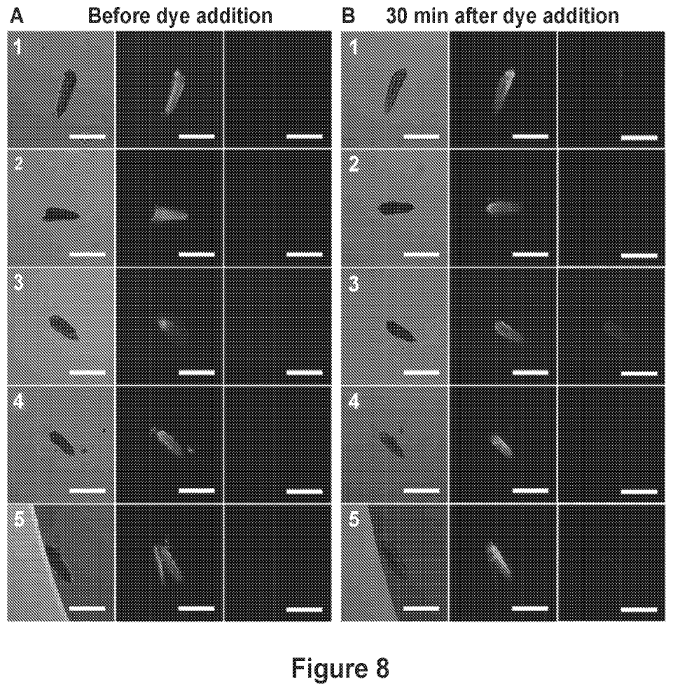

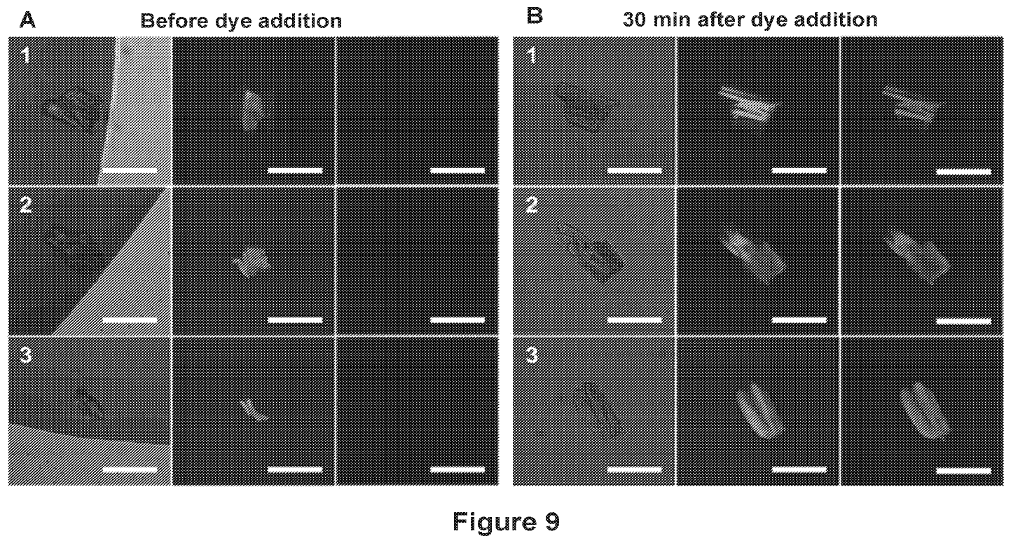

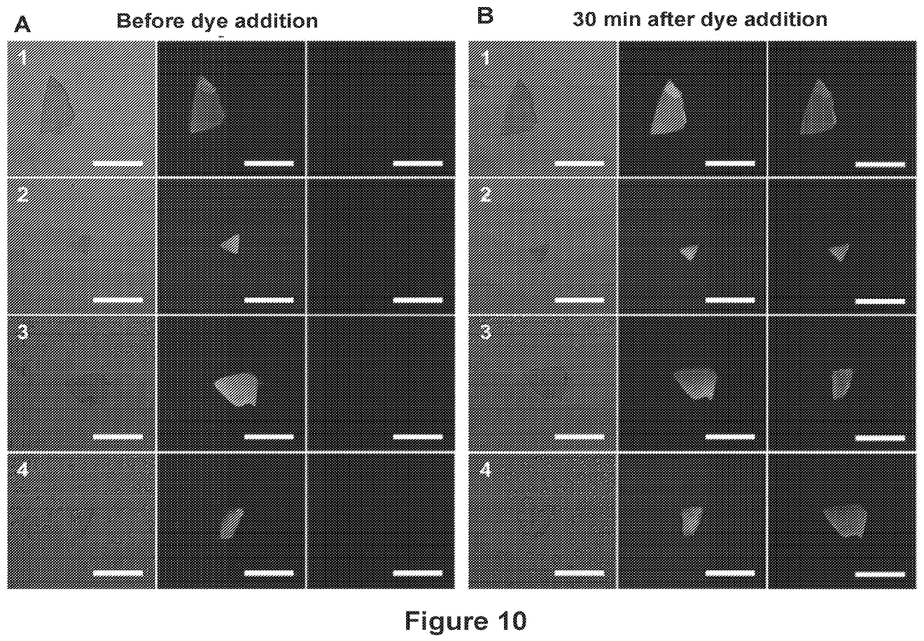

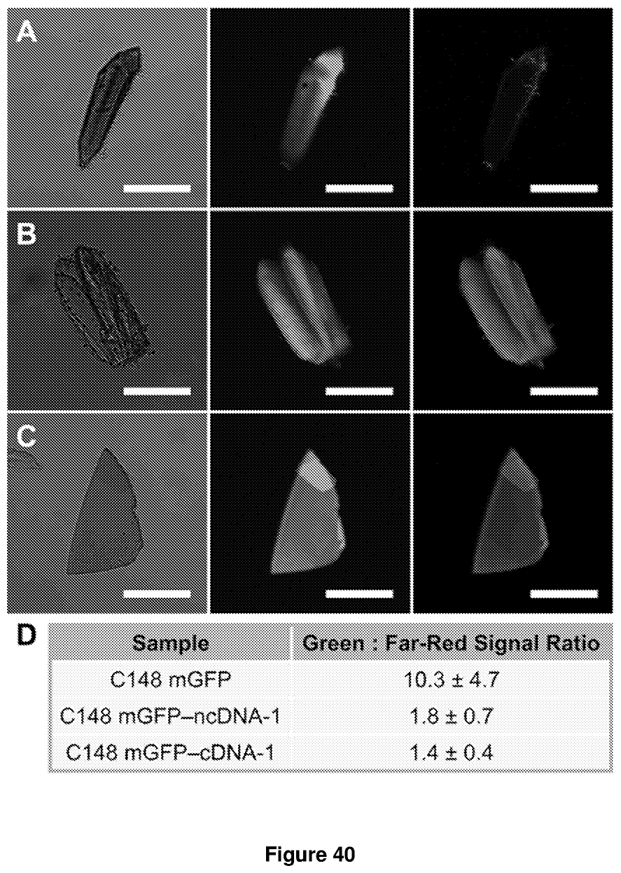

Since no direct evidence of electron density for DNA was observed in the electron density maps for the mGFP-DNA crystals structures, to confirm the presence of the DNA, crystals were incubated with the DNA-intercalating dye TOTO-3 and imaged using confocal microscopy. TOTO-3 is a cationic, DNA duplex-sensitive dye that shows a several thousand-fold increase in fluorescence upon DNA intercalation due to decreased rotational freedom, which enforces a planar conformation [Nygren, J., Svanvik, N., and Kubista, M. (1998). The interactions between the fluorescent dye thiazole orange and DNA. Biopolymers 46, 39-51; Rye, H.S., Yue, S., Wemmer, D. E., Quesada, M. A., Haugland, R. P., Mathies, R. A., and Glazer, A. N. (1992). Stable fluorescent complexes of double-stranded DNA with bis-intercalating asymmetric cyanine dyes: properties and applications. Nucleic Acids Res. 20, 2803-2812]. Before dye addition, crystals of C148 mGFP, C148 mGFP—ncDNA-1, and C148 mGFP-cDNA-1 show mGFP fluorescence (485 nm excitation and 500 — 550 nm emission filter), but no TOTO-3 fluorescence (640 nm excitation and 663 — 738 nm emission filter) (FIGS. 8-10). When TOTO-3 was added to crystals of C148 mGFP, as expected, no TOTO-3 fluorescence was observed because the mGFP crystals do not contain DNA (FIG. 40a). In contrast, a strong TOTO-3 fluorescence was observed for mGFP—ncDNA-1 (FIG. 40B) and mGFP-cDNA-1 crystals (FIG. 40c), providing evidence for the presence of DNA within the crystals of mGFP—ncDNA-1 and mGFP-cDNA-1. Surprisingly, no significant difference in the ratio of mGFP to TOTO-3 fluorescence was observed between mGFP—ncDNA-1 and mGFP-cDNA-1 crystals (FIG. 40d). While TOTO-3 is duplex-sensitive in solution, the behavior of TOTO-3 in the protein crystals is less understood [Nygren, J., Svanvik, N., and Kubista, M. (1998). The interactions between the fluorescent dye thiazole orange and DNA. Biopolymers 46, 39-51; Rye, H. S., Yue, S., Wemmer, D. E., Quesada, M. A., Haugland, R. P., Mathies, R. A., and Glazer, A. N. (1992). Stable fluorescent complexes of double-stranded DNA with bis-intercalating asymmetric cyanine dyes: properties and applications. Nucleic Acids Res. 20, 2803-2812]. In this case, it is possible that TOTO-3 dye could interact with confined single stranded DNA in the protein crystals in a way that enforces planarity and induces fluorescence. Overall, the evidence for the presence of DNA in mGFP—ncDNA and mGFP—(s)cDNA crystals from the microscopy experiment, when combined with crystallographic evidence that DNA complementarity determines crystallization outcomes, showed that protein packing in single crystals can be modulated by DNA hybridization interactions.

DNA Interaction Length Influences mGFP-DNA Packing

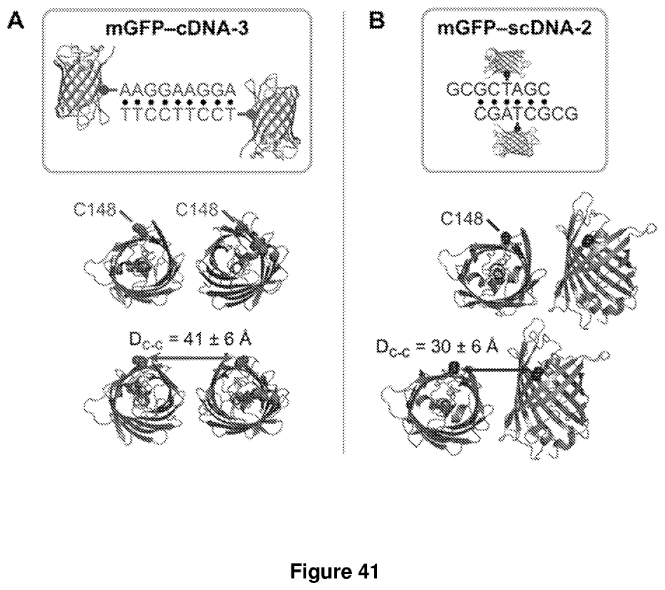

Since complementary DNA interactions can direct protein crystallization, it was next determined if DNA length provides another parameter for affecting crystal packing arrangements. To investigate the effect of DNA interaction length on crystallization outcome, DNA interactions at various lengths (6, 9, 12, and 18 bp) were designed, and mGFP-DNA conjugates incorporating these interactions were synthesized. While a single crystal form was observed for three DNA duplexes of 6 bp, an increase in DNA duplex length to 9 bp (mGFP—cDNA-3, Table 9: Line 9) led to a novel 2.9 Å structure in the space group C2 (6UHQ, FIG. 41a). The protein packing within this structure is distinct from other mGFP-DNA structures and, importantly, pairs of C148 residues again orient towards distinct regions of solvent space, separated by 41±6 A, a distance that agrees with the length of the duplex DNA (theoretical distance for 9 bp duplex is 37-75 A, either in the contracted/extended form with respect to the two alkyl linker molecules). However, when longer DNA ligands (12 bp, mGFP-cDNA-4, Table 9: Line 10 and 18 bp, mGFP-cDNA-5, Table 9: Line 11) were investigated, no crystallization was observed. This suggested that above an upper threshold for DNA duplex length, DNA is no longer able to influence the formation of mGFP single crystals. Similarly, increasing the length of non-complementary DNA from 6 to 9 bases (mGFP—ncDNA-2, Table 9: Line 12) precluded crystallization. Taken together, mGFP-DNA crystallization and structural outcomes depend strongly on the length of designed DNA.

Protein—DNA Attachment Position Influences mGFP-DNA Packing

In addition to exploring how DNA design can influence crystal structures, protein—DNA attachment position represents another powerful design parameter, where changing attachment location can guide new sets of protein-protein interactions and therefore protein packing. The amino acid attachment position was varied by changing the location of the cysteine greater than 15 Å from the middle of the side of the mGFP β-barrel (C148 mGFP) to the edge of the mGFP β-barrel (C176 mGFP and C191 mGFP). The C176 mGFP and C191 mGFP were functionalized with scDNA-1 (C176 mGFP—scDNA-1, Table 9: Line 13 and C191 mGFP—scDNA-1, Table 9: Line 14), the same DNA which directed the crystallization and structure of C148 mGFP—scDNA-1. In contrast, C176 mGFP—scDNA-1 and C191 mGFP—scDNA-1 conjugates did not crystallize, perhaps due to the high flexibility of loops at the edge of the mGFPβ-barrel. These results exhibited the importance of amino acid attachment position on crystallization outcomes.

Next, DNA base attachment position was changed from an external to an internal DNA base, which allows shorter inter-protein distances. Additionally, DNA strands with an internal base attachment position may be designed with short sticky end overhangs, which can lead to DNA ordering in single crystals [Ohayon, Y. P., Hernandez, C., Chandrasekaran, A. R., Wang, X., Abdallah, H. O., Jong, M. A., Mohsen, M. G., Sha, R., Birktoft, J. J., Lukeman, P. S., et al. (2019). Designing Higher Resolution Self-Assembled 3D DNA Crystals via Strand Terminus Modifications. ACS Nano 13, 7957-7965; Mou, Y., Yu, J.-Y., Wannier, T. M., Guo, C.-L., and Mayo, S. L. (2015). Computational design of co-assembling protein-DNA nanowires. Nature 525, 230-233]. The C148 mGFP was functionalized with a 6 bp self-complementary DNA strand with a 2 base sticky end (C148 mGFP—scDNA-2, Table 9: Line 15) and this conjugate crystallized into a new crystal form in the space group P212121 (FIG. 41b, 6UHR). Similar to other mGFP-DNA crystal structures, pairs of cysteines orient towards distinct regions of solvent space at a distance (30±6 Å) that agrees with the length of the duplex DNA (theoretical distance for 8 bp duplex with internal attachment position is 8-45 Å), further confirming that DNA interactions can be extensively designed to influence the crystallization and packing of proteins. This structure suggested an additional layer of control provided by the DNA ligand including linker flexibility and sticky end design.

### CONCLUSIONS

Growth of protein single crystals involves complex protein—protein interactions which are challenging to design and predict. The foregoing examples demonstrated how replacing such interactions with highly programmable DNA interactions enables structural control over protein packing within single crystals. The first protein single crystal structure where DNA hybridization interactions between the surfaces of proteins direct the packing of proteins within the crystal is reported herein. Furthermore, that DNA complementarity, DNA length, and protein—DNA attachment position all influence crystallization and protein packing structural outcomes has been demonstrated. The resulting crystal structure was shown to be independent of DNA sequence (while maintaining complementarity), and for the mGFP-DNA conjugates crystallization only occurred when DNA duplexes were less than or equal to 9 bp. Interestingly, changing the DNA length or the attachment of DNA to the protein through an internal base modification afforded more novel crystal structures that further demonstrated the versatility of this approach and the large design space to be explored. Together, the work presented herein is an essential step towards designing and engineering protein packing within single crystals.

### REFERENCES

1. Hollingsworth, M. D., Science, 2002, 295, 2410-2413.

2. Desiraju, G. R., Angew. Chem. Int. Ed., 2007, 46, 8342-8356.

3. Yaghi, O. M., O'Keefe, M., Ockwig, N. W., Chae, H. K., Eddaoudi, M., and Kim, J., Nature, 2003, 423, 705-714.

4. Feng, X., Ding, X., and Jiang, D., Che. Soc. Rev., 2012, 41, 6010-6022.

5. Khalaf, N., Govardhan, C. P., Lalonde, J. J., Persichetti, R. A., Wang, Y., and Margolin, A. L., J. Am. Chem. Soc., 1996, 118, 5494-5495.

6. Resenbaum, D. M, Cherezov, V., Hanson, M. A., Rasmussen, S. G. F., Thian, F. S., Koblika, T. S., Choi, H., Yao, X., Weis, W. I., Stevens, R. C., and Kobilka, B. K., Science, 2007, 318, 1266-1273.

7. Lalonde, J. J., Govardhan, C., Khalaf, N., Martinez, A. G., Kalevi, V., and Margolin, A. L., J. Am. Chem. Soc., 1995, 117, 6845-6852.

8. McPherson, A. and Gavira, J. A., Acta Cryst. F, 2014, 70, 2-20.

9. Eddaoudi, M., Moler, D. B., Li, H., Chen, B., Reineke, T. M., O'Keeffe, M., and Yaghi, O. M., Acc. Chem. Res., 2001, 34, 319-330.

10. Gonen, S., DiMaio, F., Gonen, T., and Baker, D., Science, 2015, 348, 1365-1368.

11. Rothemund, P. W. K., Nature, 2006, 440, 297-302.

12. Seeman, N. C., Nature, 2003,421, 427-431.

13. Seeman, N. C., J. Theor. Biol., 1982, 99, 237-247.

14. Mirkin, C. A., Letsinger, R. L., Mucic, R. C., and Storhoff, J. J., Nature, 1996, 382, 607-609.

15. Macfarlane, R. J., Lee, B., Jones, M. R., Harris, N., Schatz, G. C., and Mirkin, C. A., Science, 2011, 334, 204-208.

16. Hayes, O. G., McMillan, J. R., Lee, B., and Mirkin, C. A., J. Am. Chem. Soc., 2018, 140, 9269-9274.

17. Brodin, J. D., Auyeung, E., and Mirkin, C. A., Proc Natl Acad Sci, 2015, 112, 4564-4569.

18. McMillan, J. R., Brodin, J. D.; Millan, J. A., Lee, B., Olvera de la Cruz, M., and Mirkin, C. A., J. Am. Chem. Soc., 2017, 139, 1754-1757.

19. McMillan, J. R., and Mirkin, C. A., J. Am. Chem. Soc., 2018, 140, 6776-6779.

20. McMillan, J. R., Hayes, 0. G., Remis, J. P., and Mirkin, C. A., J. Am. Chem. Soc., 2018, 140, 15950-15956.

21. MacDonald, J. I., Munch, H. K., Moore, T., and Francis, M. B., Nat. Chem. Biol., 2015, 11, 326-331.

22. Subramanian, R. H., Smith, S. J., Alberstein, R. G., Bailey, J. B., Zhang, L., Cardone, G., Suominen, L., Chami, M., Stahlberg, H., Baker, T. S., and Tezcan, F. A., ACS Cent. Sci., 2018, Articles ASAP.

23. Bruggink, A., Schoevaart, R., and Kieboom, T., Org. Proc. Res. Dev., 2003, 7, 622-640.

24. Mattiasson, B. and Mosbach, K., Biochim. Biophys. Acta, Protein Struct. Mol. Enzymol., 1971, 235, 253-257.

25. Fu, J., Liu, M., Liu, Y., Woodbury, N. W., and Yan, H. J. Am. Chem. Soc., 2012, 134, 5516-5519.

26. Schoedel, A., Li, M., Li, D., O'Keeffe, M., and Yaghi, O. M., Chem. Rev., 2016, 116, 12466-12535.

27. Khalaf, N., Govardhan, C. P., Lalonde, J. J., Persichetti, R. A., Wang, Y., and Margolin, A. L., J. Am. Chem. Soc., 1996, 118, 5494-5495.

28. Schrittwieser, J. H., Lavandera, I., Seisser, B., Mautner, B., and Kroutil, W., Eur. J. Org. Chem., 2009, 2009, 2293-2298.

What is claimed is:

1. A method of producing a protein crystal comprising:
contacting a first conjugate comprising a first protein and a first polynucleotide with a second conjugate comprising a second protein and a second polynucleotide under conditions sufficient such that the first polynucleotide and the second polynucleotide hybridize to each other and the first protein and second protein associate via protein-protein interactions (PPI) to form the protein crystal.

2. The method of claim 1, wherein the first protein and the second protein are the same.

3. The method of claim 1, wherein the first protein and the second protein are different.

4. The method of any one of the preceding claims, wherein the first polynucleotide is from about 2 to about 30 nucleotides in length.

5. The method of any one of the preceding claims, wherein the second polynucleotide is from about 2 to about 30 nucleotides in length.

6. The method of any one of the preceding claims, wherein the first protein consists of one polynucleotide that is sufficiently complementary to one or more polynucleotides on the second protein to hybridize.

7. The method of any one of the preceding claims, wherein the first protein consists of two, three, four, or five polynucleotides that are sufficiently complementary to one or more polynucleotides on the second protein to hybridize.

8. The method of any one of the preceding claims, wherein the second protein consists of one polynucleotide that is sufficiently complementary to one or more polynucleotides on the first protein to hybridize.

9. The method of any one of the preceding claims, wherein the second protein consists of two, three, four, or five polynucleotides that are sufficiently complementary to one or more polynucleotides on the first protein to hybridize.

10. The method of any one of the preceding claims, wherein the PPI is a hydrophobic bond, van der Waals forces, a salt bridge, a disulfide bond, an electrostatic interaction, hydrogen bonding, or a combination thereof.

11. The method of any one of the preceding claims, wherein the protein crystal is from about 250 nanometer (nm) to about 1 millimeter (mm), or from about 20 micrometers (pm) to about 500 μm in edge length.

12. The method of any one of the preceding claims, wherein structure of the protein crystal diffracts to angstrom level resolution.

13. The method of any one of the preceding claims, wherein the first polynucleotide is attached to the N-terminus of the first protein.

14. The method of any one of the preceding claims, wherein the second polynucleotide is attached to the N-terminus of the second protein.

15. The method of any one of the preceding claims, wherein the first polynucleotide is attached to the first protein via an unnatural amino acid introduced into the first protein via mutation.

16. The method of any one of the preceding claims, wherein the second polynucleotide is attached to the second protein via an unnatural amino acid introduced into the second protein via mutation.

17. The method of any one of the preceding claims, wherein the first polynucleotide is attached to the first protein via a surface amino group of the first protein.

18. The method of any one of the preceding claims, wherein the second polynucleotide is attached to the second protein via a surface amino group of the second protein.

19. The method of claim 17 or claim 18, wherein the surface amino group is from a Lys residue.

20. The method of any one of claims 17-19, wherein the first polynucleotide is attached to the first protein via a triazole linkage formed from reaction of (a) an azide moiety attached to the surface amino group and (b) an alkyne functional group on the first polynucleotide.

21. The method of any one of claims 17-20, wherein the second polynucleotide is attached to the second protein via a triazole linkage formed from reaction of (a) an azide moiety attached to the surface amino group and (b) an alkyne functional group on the second polynucleotide.

22. The method of any one of the preceding claims, wherein the first polynucleotide is attached to the first protein via a surface carboxyl group of the first protein.

23. The method of any one of the preceding claims, wherein the second polynucleotide is attached to the second protein via a surface carboxyl group of the second protein.

24. The method of any one of the preceding claims, wherein the first polynucleotide is attached to the first protein via a surface thiol group of the first protein.

25. The method of any one of the preceding claims, wherein the second polynucleotide is attached to the second protein via a surface thiol group of the second protein.

26. The method of any one of the preceding claims, wherein the protein crystal exhibits catalytic, signaling, therapeutic, or transport activity.

27. The method of any one of the preceding claims, wherein the first protein and/or the second protein is a protein fragment.

28. The method of any one of the preceding claims, wherein the contacting step further comprises contacting the first conjugate and/or the second conjugate with a third conjugate comprising a third protein and a third polynucleotide, wherein the third polynucleotide hybridizes to the first polynucleotide or the second polynucleotide, and the resulting protein crystal comprises the first protein, second protein, and third protein.

29. The method of any one of the preceding claims, wherein the protein crystal has a pore size of about 1 nanometer (nm)-100 nm in diameter.

30. A protein crystal comprising a first conjugate and a second conjugate, wherein the first conjugate comprises a first protein and a first polynucleotide and the second conjugate comprises a second protein and a second polynucleotide, wherein the first polynucleotide and the second polynucleotide are sufficiently complementary to hybridize to each other.

31. The protein crystal of claim 30, wherein the first protein and the second protein are the same.

32. The protein crystal of claim 30, wherein the first protein and the second protein are different.

33. The protein crystal of any one of claims 30-32, wherein the first polynucleotide is from about 2 to about 30 nucleotides in length.

34. The protein crystal of any one of claims 30-33, wherein the second polynucleotide is from about 2 to about 30 nucleotides in length.

35. The protein crystal of any one of claims 30-34, wherein the first protein consists of one, two, three, four, or five polynucleotides that are sufficiently complementary to one or more polynucleotides on the second protein to hybridize.

36. The protein crystal of any one of claims 30-35, wherein the second protein consists of one, two, three, four, or five polynucleotides that are sufficiently complementary to one or more polynucleotides on the first protein to hybridize.

37. The protein crystal of any one of claims 30-36, wherein the first protein and the second protein associate with each other through a protein-protein interaction (PPI).

38. The protein crystal of claim 37, wherein the PPI is a hydrophobic bond, van der Waals forces, a salt bridge, a disulfide bond, an electrostatic interaction, hydrogen bonding, or a combination thereof.

39. The protein crystal of any one of claims 30-38, wherein the protein crystal is from about 250 nanometer (nm) to about 1 millimeter (mm), or from about 20 micrometers (pm) to about 500 μm in edge length.

40. The protein crystal of any one of claims 30-39, wherein structure of the protein crystal diffracts to angstrom level resolution.

41. The protein crystal of any one of claims 30-40, wherein the first polynucleotide is attached to the N-terminus of the first protein.

42. The protein crystal of any one of claims 30-41, wherein the second polynucleotide is attached to the N-terminus of the second protein.

43. The protein crystal of any one of claims 30-42, wherein the first polynucleotide is attached to the first protein via an unnatural amino acid introduced into the first protein via mutation.

44. The protein crystal of any one of claims 30-43, wherein the second polynucleotide is attached to the second protein via an unnatural amino acid introduced into the second protein via mutation.

45. The protein crystal of any one of claims 30-44, wherein the first polynucleotide is attached to the first protein via a surface amino group of the first protein.

46. The protein crystal of any one of claims 30-45, wherein the second polynucleotide is attached to the second protein via a surface amino group of the second protein.

47. The protein crystal of claim 45 or claim 46, wherein the surface amino group is from a Lys residue.

48. The protein crystal of any one of claims 45-47, wherein the first polynucleotide is attached to the first protein via a triazole linkage formed from reaction of (a) an azide moiety attached to the surface amino group and (b) an alkyne functional group on the first polynucleotide.

49. The protein crystal of any one of claims 45-48, wherein the second polynucleotide is attached to the second protein via a triazole linkage formed from reaction of (a) an azide moiety attached to the surface amino group and (b) an alkyne functional group on the second polynucleotide.

50. The protein crystal of any one of claims 30-49, wherein the first polynucleotide is attached to the first protein via a surface carboxyl group of the first protein.

51. The protein crystal of any one of claims 30-50, wherein the second polynucleotide is attached to the second protein via a surface carboxyl group of the second protein.

52. The protein crystal of any one of claims 30-51, wherein the first polynucleotide is attached to the first protein via a surface thiol group of the first protein.

53. The protein crystal of any one of claims 30-52, wherein the second polynucleotide is attached to the second protein via a surface thiol group of the second protein.

54. The protein crystal of any one of claims 30-53, wherein the protein crystal exhibits catalytic, signaling, therapeutic, or transport activity.

55. The protein crystal of any one of claims 30-54, wherein the first protein and/or the second protein is a protein fragment.

56. The protein crystal of any one of claims 30-55, further comprising a third conjugate comprising a third protein and a third polynucleotide, wherein the third polynucleotide is sufficiently complementary to the first polynucleotide or the second polynucleotide to hybridize.

57. The protein crystal of any one of claims 30-56, wherein the protein crystal has a pore size of from about 1 nanometer (nm) to about 100 nm in diameter.

58. A method of catalyzing a reaction comprising contacting one or more reagents for the reaction with the protein crystal of any one of claims 30-57, wherein contact between the reagents and the protein crystal results in the reaction being catalyzed to form a product of the reaction.


SEQUENCE LISTING (7 sequences)

SEQ ID NO: 1 - protein, 274 aa, Artificial Sequence
  Synthetic
MRGSHHHHHHGMASMTGGQQMGRDLYENLYDDDDKMVSKGEELFTGVVPILVELDGDVNGHKFSVSGEGEGDATYGKLTLKFILTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNCHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 2 - protein, 274 aa, Artificial Sequence
  Synthetic
MRGSHHHHHHGMASMTGGQQMGRDLYENLYDDDDKMVSKGEELFTGVVPILVELDGDVNGHKFSVSGEGEGDATYGKLTLKFILTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGCVQLADHYQQNTPIGDGPVLLPDNHYLSTQSALSKDPNEKRDHMVLLEFVTAAGITLGMDELYK

SEQ ID NO: 3 - protein, 274 aa, Artificial Sequence
  Synthetic
MRGSHHHHHHGMASMTGGQQMGRDLYENLYDDDDKMVSKGEELFTGVVPILVELDGDVNGHKFSVSGEGEGDATYGKLTLKFILTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGCGPVLLPDNHYLSTQSALSKDPNEKRDHMVLLEFVTAAGITLGMDELYK

SEQ ID NO: 4 - DNA, 12 bp, Artificial Sequence
  Synthetic
  misc_feature (1)..(1): H2N Modifer
aaggaaggaagg

SEQ ID NO: 5 - DNA, 12 bp, Artificial Sequence
  Synthetic
  misc_feature (1)..(1): H2N Modifier
ccttccttcctt

SEQ ID NO: 6 - DNA, 18 bp, Artificial Sequence
  Synthetic
  misc_feature (1)..(1): H2N Modifier
agttaggacttacgctac

SEQ ID NO: 7 - DNA, 18 bp, Artificial Sequence
  Synthetic
  misc_feature (1)..(1): H2N Modifier
gtagcgtaagtcctaact